(19) United States
(12) Patent Application Publication
(10) Pub. No.: US 2008/0274558 A1
(43) Pub. Date: Nov. 6, 2008

(54) METHOD FOR IDENTIFYING AND SELECTING LOW COPY NUCLEIC SEGMENTS

(75) Inventors: Heather Newkirk, Kansas City, MO (US); Chengpeng Bi, Kansas City, MO (US)

Correspondence Address: POLSINELLI SHALTON FLANIGAN SUELTHAUS PC, 700 W. 47TH STREET, SUITE 1000, KANSAS CITY, MO, 64112-1802, US

(73) Assignee: THE CHILDREN'S MERCY HOSPITAL, Kansas City, MO (US)

(21) Appl. No.: 12/058,659

(22) Filed: Mar. 28, 2008

Related U.S. Application Data
(60) Provisional application No. 60/908,606, filed on Mar. 28, 2007.
(60) Provisional application No. 60/940,321, filed on May 25, 2007.

(51) Int. Cl. G01N 33/00

(52) U.S. Cl. 436/94

(57) ABSTRACT

The present invention relates to a method of identifying low copy nucleic acid segments from within a known nucleic acid sequence and selecting among the identified low copy segments for segments that are thermodynamically suitable for use in hybridization experiments.

### RELATED APPLICATIONS

This application relates to and claims priority to U.S. Provisional Patent Application No. 60/908,606, which was filed Mar. 28, 2007 and to U.S. Provisional Patent Application No. 60/940,321, which was filed May 25, 2007. Both of which are incorporated herein by reference in their entireties.

All applications are commonly owned.

### SEQUENCE LISTING

This application contains a sequence listing submitted in electronic format in compliance with 37 C.F.R. 1.821-1.825 and in compliance with the EFS-Web requirements. This sequence listing is incorporated herein by reference in its entirety.

### BACKGROUND OF THE INVENTION

1. Field of the Invention

The present invention relates to a method of identifying low copy nucleic acid segments, suitable for use in hybridization experiments, from within a known nucleic acid sequence. The present invention further relates to a method of preferentially selecting among the identified low copy nucleic acid segments for segments that are thermodynamically suitable for use in hybridization experiments.

2. Description of the Prior Art

Use of low copy number probes to target homologous segments on nucleic acid sequences is known in the prior art. Some prior art methods have relied on scanning a target sequence segment against a database of repetitive sequences, whereby probe sequences were identified as lying between two adjacent repetitive sequences. However, such methods were only as reliable as the quality of the database of repetitive sequences. Moreover, some probe sequences identified by such methods were unsuitable for hybridization due, for example, to secondary structural conformations (e.g. hairpin loops, stems, bulges, etc.). Other methods for identifying low copy number nucleic acid segments for use as probes have involved a laborious process that typically requires considerable review and analysis at multiple steps by a knowledgeable researcher.

Computer methods commonly used to identify unique sequence regions include web-based programs such as Repeat Masker (publicly available on the world wide web at a website that reads in pertinent part “repeatmasker.org”) and BLAT (publicly available on the world wide web at a website that reads in pertinent part “genome.ucsc.edu”). Neither of these programs evaluates genomic sequences for thermodynamic characteristics of genomic regions. Accordingly, probes extracted from these programs can contain unique sequences; however, such sequences may not be suitable for hybridization. Presently, a determination of whether such sequences are suitable for hybridization requires that the sequences be physically made into probes or primers, which is generally time and cost consuming.

Computer methods used to assess the thermodynamic qualities of a potential probe sequence are not capable of initially identifying the sequence. For example, a commonly used program for thermodynamic assessment of genomic sequences, Mfold (publicly available on the world wide web at a website that reads in pertinent part “bioinfo.rpi.edu”), does not evaluate genomic sequences for their unique sequence nature. As such, a user cannot be certain that the thermodynamically stable sequence that has been identified will be unique until tested. Since testing a probe consumes both time and money, it is desired to find a more reliable method of identifying thermodynamically stable, unique sequences within a genetic segment.

Accordingly, what is needed in the art is a method for quickly and reliably identifying low copy number nucleic acid segments, suitable for hybridization, from known nucleic acid sequences. Further, what is needed is a method of quickly identifying, from a known nucleic acid sequence of extended length, low copy nucleic acid segments that are thermodynamically suitable for hybridization.

### SUMMARY OF THE INVENTION

The present invention overcomes the problems inherent in the prior art and provides a distinct advance in the state of the art by providing methods and computerized processes for the rapid and reliable identification of low copy nucleic acid segments from within a known nucleic acid sequence and for the selection from the identified low copy segments of segments that are thermodynamically suitable for use in hybridization experiments.

The invention advantageously provides for greater sensitivity and higher throughput in hybridization. The methods allow the user to analyze longer sequence lengths at a time versus other genomics programs, while still being capable of analyzing sequences of any length. These longer sequences may be greater than 100 kilobases (kb), 150 kb, 200 kb, 250 kb, 300 kb, 500 kb, or even 1000 kb or more in length. In addition, the parameters used by this method are stricter than those commonly used on web-based programs. These strict criteria, including ΔG (Gibbs Free Energy), ΔH (Enthalpy), ΔS (Entropy), and Tm (Melting Temperature), based on the Gibb's Free Energy Equation, allow for the highly efficient selection of only unique sequence probes for use in genomic experiments. It is understood that the Gibb's Free Energy Equation is an equation and the variables ΔH, ΔS, and Tm can be manipulated in order to arrive at the desired ΔG, which is <50 in preferred forms. If manipulation of 1 or more of these variables is outside of the preferred range but still results in a ΔG<50, these criteria or parameters are also covered by the present invention. In preferred forms, the criteria or parameters will require that ΔG<50, ΔH<−1000, ΔS<−3500, Tm≧60 C. For QMH, these are the most preferred criteria or parameters; for FISH, the most preferred Tm is ≧42 C; and for array-based technologies, the most preferred Tm≧37 C.

Methods of the invention are more comprehensive, compared to present technologies, because they combine sequence analysis with thermodynamic analysis to identify nucleic acid segments that are both low copy sequences (i.e. not repetitive sequences, and preferably single copy meaning that the sequence appears only a single time in the genome) and thermodynamically suitable for hybridization. Additionally, methods of the invention identify unique sequences and search the genome to ensure that no other non-repetitive genomic regions are homologous to the region of interest. Further, unlike technology in the art, methods of the invention provide a double-check analysis of low copy nucleic acid segments to determine their suitability to be used as primers for polymerase chain reaction (PCR), or in other techniques that rely on variable temperatures. This represents the first invention to use such analytical methods sequentially.

This invention is quite versatile in that it can be employed to design a variety of low copy nucleic acid probes of different lengths with characteristics that can be user-defined. For example, the present invention allows the user to choose the length of a unique sequence probe for the output.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein. The application contains at least one drawing executed in color. Copies of this patent application publication with color drawing(s) will be provided by the Office upon request and payment of the necessary fee.

FIG. 1 is a screen capture showing an input screen for the web-based Unique Genomic Sequence Hunter (UGSH) program;

FIG. 2A is a screen capture showing exemplary output from UGSH displaying unique sequence genomic probes and locations. FIG. 2B is a screen capture showing an exemplary Primer Selection Output screen from UGSH. FIG. 2C is a screen capture showing an exemplary primer sequence file from UGSH displayed in FASTA format;

FIG. 3 is a photograph taken from a fluorescence in situ hybridization (FISH) experiment using a unique sequence probe from BAC RP11-677F14 on chromosome 7;

FIG. 4 is a photograph taken from a FISH experiment using a unique sequence probe cocktail containing five, different unique sequence probes;

FIG. 5 illustrates the results of a FISH experiment, using a probe not designed using the UGSH method. Probes (light gray, arrows) hybridized to numerous chromosomal locations, indicating that this sequence is homologous to more than one chromosomal region and thus not comprising a purely unique sequence;

FIG. 6 is a flow chart illustrating an embodiment of a computerized method for identifying low copy nucleic acid segments from within a known nucleic acid sequence, and selecting among the identified low copy segments for segments that are thermodynamically suitable for use in hybridization experiments;

FIG. 7 is a flow chart illustrating a further embodiment of a computerized method for identifying low copy nucleic acid segments from within a known nucleic acid sequence and selecting among the identified low copy segments for segments that are thermodynamically suitable for use in hybridization experiments;

FIG. 8 is a flow chart illustrating an embodiment of a computerized method for identifying known repetitive sequences within an exemplary sequence from a subject or patient; and

FIG. 9 is a flow chart illustrating an embodiment of a computerized method for extracting known repetitive sequences from a sequence from a subject or patient and selecting remaining portions of the sequence according to user-specified size parameters.

### DETAILED DESCRIPTION

The present invention comprises a new, computerized process for the identification of unique sequence regions in genomic DNA, and provides methods to design unique-sequence genomic segments. The identified segments can in turn be synthesized or amplified from a genome, or part of a genome, genomic library, or other source of genomic DNA and utilized in hybridization experiments such as, but not limited to, microarray, arrayCGH (collectively with microarray termed “array-based”), quantitative microsphere hybridization (QMH), and fluorescent in situ hybridization (FISH). The computerized process and associated methods return only sequences matching the users criteria (for example, displayed within a computer program window, stored in a data file, printout, or other output), and sequences not meeting the criteria are discarded.

These methods are an improvement over previous methods since genomic sequences, or segments, are evaluated for unique, or non-repetitive, sequence composition by combining two different strategies and analyzing the thermodynamic characteristics of any identified unique sequence regions to ensure optimal performance of an identified low copy nucleic acid segment in hybridization assays.

The methods presented here offer an advancement over present technology by analyzing sequences for both their genomic representation, i.e. distribution, as well as their thermodynamic properties using a single computer program, referred to herein as Unique Genomic Sequence Hunter (UGSH). A preferred form of this method includes five main steps: 1) Removing highly and moderately repetitive sequences from a sequence of interest and displaying those genomic segments (i.e. the segments remaining after the repetitive sequences are removed). These resulting genomic segments can be of any size, but for FIS, they are preferably greater than 500 bp, more preferably greater than 750 bp, and most preferably greater than 1 kb; 2) Searching each segment for homology to genomic regions other than the region of interest and discarding all segments which match elsewhere in the genome; 3) Evaluating unique sequence segments for possible secondary structure motifs (hairpin loops, stems, bulges, etc.) by thermodynamic analysis; 4) Designing PCR primers for genomic segments which pass the above three steps; and, 5) evaluating each PCR primer to ensure it contains only unique sequence and does not match elsewhere in the genome. In some preferred forms, the process stops after step 3, and in other preferred forms, the process stops after step 4. However, in use, it is preferred to perform all 5 steps.

This series of steps offers a more robust and accurate tool for designing unique sequence probes for use in genomic laboratory experiments. Steps do not necessarily need to occur in the aforestated sequential order. In variations of this basic method, one or more of the above steps are eliminated. In an exemplary embodiment, multiple steps in the method are automated via computer program. Preferably, the computer program is written in a computer language well-adapted for creating web-based applications, such as Perl.

### Development of UGSH

The UGSH method was developed through the iterative design and experimental testing of genomic probes. Initially, methods from the prior art (U.S. Pat. Nos. 6,828,097 ('097 patent) and 7,014,997 ('997 patent)) were used for the generation of “single copy” probes for quantitative microsphere hybridization (QMH) experiments (Newkirk et al. 2006, Determination of genomic copy number with quantitative microsphere hybridization. Human Mutation 27:376-386). The QMH assay allows for the high-throughput determination of genomic copy number by the direct hybridization of unique sequence probes, attached to spectrally distinct microspheres, to biotinylated genomic patient DNA, followed by flow cytometric analysis (Newkirk et al. 2006, U.S. Provisional Patent Application Ser. No. 60/708,734). During flow cytometry, the mean fluorescence intensity (MFI) is measured for a test probe and a reference probe, known to be present in two copies per diploid genome, in a multiplex reaction. MFI ratios (test:reference) are subsequently calculated to discern whether the test probe is present in two copies (MFI ratio=1), one copy (MFI ratio=0.5), or more than two copies (MFI ratio>1). Step 1, as described above, of the UGSH method is similar but distinct from the methods described in the aforesaid patent applications. Methods of the aforesaid patent applications involve repeat-masking (i.e. running a comparison of the sequence of interest with all known repetitive sequences in a genome and eliminating or “masking” those sequences that have 90% or higher sequence similarity (which can introduce gaps and windows to provide a better match between two sequences)) a sequence of interest to generate unique or “single copy probes”. For example, after analyzing a sequence specific to ABL1 (chr9) using the method of '097 patent, a probe was designed (designated, ABLA1uMer1) for QMH (Newkirk et al. 2005). A known single copy HOXB1 sequence (Newkirk et al., 2006) was used as the reference sequence. Both probes (˜100 bases) were coupled to spectrally distinct microspheres and hybridized to biotinylated normal control genomic DNA. The MFI ratio of the HOXB1 and ABLA1uMer1 probe should be 1 since a normal control DNA was used for validation, however the MFI ratio was 4.55 indicating that the ABLA1uMer1 sequence hybridized to other homologous regions in the genome (Newkirk et al., 2005, Distortion of quantitative genomic and expression hybridization by Cot-1 DNA: mitigation of this effect. Nucleic Acids Research 33:e191).

A different strategy was then used which involved repeat-masking (Step 1) followed by a genomic homology search (Step 2) and probe 16-1d was designed specific to ABL (Newkirk et al., 2006). This probe was hybridized to two different normal human genomic DNAs in QMH reactions with HOXB1 and yielded respective MFI ratios of 1.36 and 1.18. While closer to 1, these ratios are still not optimal. Subsequent analysis of the 16-1d probe revealed a stable hairpin loop structure close to the 3′ end of the probe (Newkirk et al., 2006), which could account for its less-than-optimal MFI ratios. To further improve the method, a secondary structure analysis step (Step 3) was integrated for refinement of the UGSH method.

After removing repeats from the ABL sequence region of interest, and performing genomic homology searches and secondary structure analysis, another probe was developed, 16-1b (100 bases, Newkirk et al., 2006). When 16-1b was used in QMH experiments with HOXB1, MFI ratios were 1.01±0.01 (16 normal samples tested), indicating that this probe was hybridizing to a single location in the genome. Thus, a combination of steps 1, 2, and 3 provided better results than were previously possible. The precise parameters for the secondary structure analysis (ΔG<50, ΔH<−1000, ΔS<−3500, Tm≧65 C if above criteria not met) were ascertained by experimentation using unique sequence probes of varying degrees of secondary structure. One developed probe of the prior art, 16-1a, revealed strong secondary structure characteristics (ΔG=−122, ΔH=−1584, ΔS=−4714, Tm=63 C) (Newkirk et al., 2006). When probe 16-1a was co-hybridized with HOXB1 in QMH reactions the MFI ratios ranged from 0.73 to 0.93 (n=4) for a normal genomic control sample, which indicated the instability of the probe. Another probe of the prior art, 16-2A, designed using repeat-masking followed by genomic homology searches (steps 1 and 2 above) also revealed rather strong secondary structure characteristics (ΔG=−91, ΔH=−1296, ΔS=−3886, Tm=60 C) (Newkirk et al., 2006).

In QMH experiments with HOXB1, the MFI ratio ranged from 0.84 to 0.92 (n=4) in QMH reactions with normal genomic DNA, indicating a little more stable probe structure with MFI ratios closer to 1. Probe 16-1b (Newkirk et al., 2006) had different secondary structure characteristics (ΔG=−9.66, ΔH=−138.8, ΔS=−416.4, Tm=60.2 C) and yielded MFI ratios between 0.96 and 1.09 (n=11) for multiplex hybridization with HOXB1 to normal genomic control DNA samples (Newkirk et al., 2006).

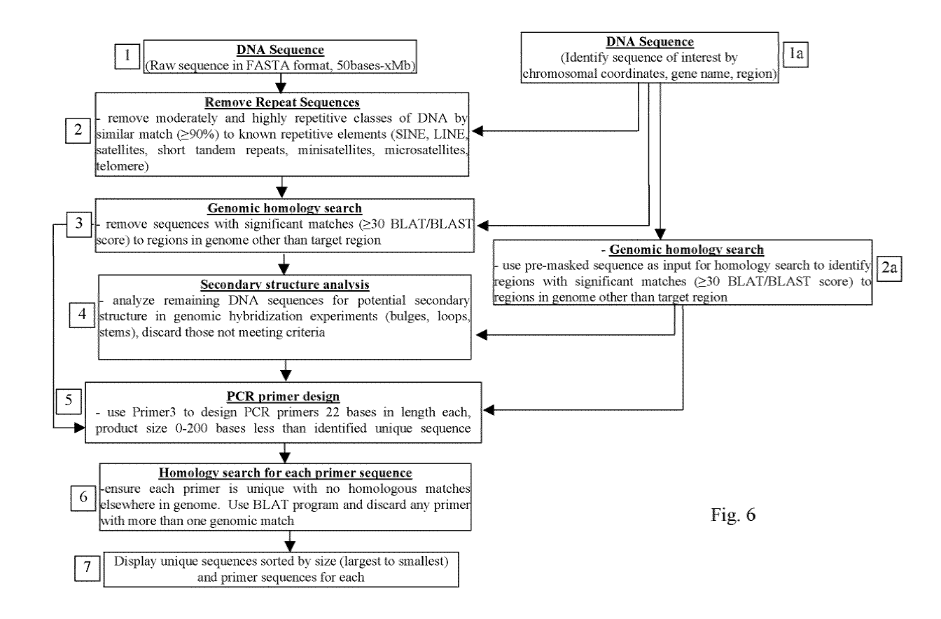

With reference to FIG. 6, the Unique Genome Sequence Hunter (UGSH) method for genomic hybridization probe selection requires a DNA sequence (step 1), which can be entered into the UGSH program in FASTA or Genbank format. Alternatively, this sequence can be defined by chromosomal coordinates, gene name, or region of interest (step 1a). In this case (step 1a), UGSH will query a database, with a particularly preferred database being the UCSC database (genome.ucsc.edu) to retrieve the appropriate sequence corresponding to the query (ie. Chr15:21263421-21263821, SNRPN, PWS, etc.). The next step in the process (step 2) is to remove repetitive sequences from the input sequence. UGSH does this by aligning the sequences of highly repetitive classes of DNA (SINE, LINE, satellites, short tandem repeats, minisatellites, microsatellites, telomere, etc.) to the sequence of interest. Specifically, UGSH runs the RepeatMasker program to remove repetitive sequences, but it uses strictly defined output parameters for Repeat Masker to eliminate all sequences with greater than or equal to a 90% homology match to known repeat sequences. Any similar repeat masking program could be used for this procedure. Alternatively, this repeat masking step can be circumvented by inputting a query sequence that is already masked for repeats (step 2A). The UCSC genomic browser and Genbank offer the option to display masked sequences, thus eliminating the need for this repeat-masking step.

At this stage in the method, the UGSH program has generated a DNA sequence that is masked for repeats. The next step in the process (step 3) is to scan this sequence for homologous sequences in the genome using the BLAT program from the UCSC genome browser. Any segment of the sequence which has a BLAT score greater than or equal to 30 is discarded from probe selection. Any genome-wide homology search program, such as BLAST from NCBI, can be substituted for BLAT and the same parameters used (acceptable score ≦30 or between 1-30, preferably less than 25 (or between 1-25), even more preferably less than 20 (or between 1-20), still more preferably less than 15 (or between 1-15), even more preferably less than 10 (or between 1-10), still more preferably less than 8 (or between 1-8), even more preferably less than 6 (or between 1-6), still more preferably less than 5 (or between 1-5), even more preferably less than 4 (or between 1-4), still more preferably less than 3 (or between 1-3), even more preferably, less than 2 (or between 1-2), and most preferably 1).

The remaining sequence that is repeat-free and has little to no homology elsewhere in the genome is then examined for potential secondary structure (i.e. bulges, loops, or stems) which could render the probe suboptimal for genomic hybridization experiments (step 4). The preferred UGSH method utilizes the Mfold program and uses strictly defined parameters (ΔG<50, ΔH<−1000, ΔS<−3500, Tm≧60° C., or as otherwise noted for QMH, or array-based applications) for probe selection. If these parameters are not met, the sequence is discarded from probe design.

The remaining sequences, after secondary structure analysis has been performed, are used for PCR primer design if PCR probes are desired (step 5). The UGSH method employs the Primer3 program (Rozen et al., 2000) to design primers at least 15 bases in length. For FISH applications, these primers can range in length from 15-100 bases; for array-based and QMH applications, these primers can range from 15-70, and more preferably from 25-70 bases in length. One particularly preferred length for FISH applications is 22 bases in length. Moreover, in all applications, the product size will be equal to or slightly less than the input sequenced size. Preferably the product size will be equal to or slightly less than 0 to 200 bases less than the input sequence size, however any conventional primer selection program could be substituted and longer input sequences could have product sizes more than 200 bases less than the input sequence size. Primers are then BLAT searched using the UCSC BLAT program (step 6) to ensure that there is no homologous sequence elsewhere in the genome. Any primer which has more than one genomic match is discarded. The PCR primer design step and PCR primer homology search step can be omitted if hybridization oligonucleotides are desired instead of PCR probes, and the repeat-free sequences with no homologous genome matches from step 4 can be used as hybridization probes. After completing all processes, UGSH then displays the unique sequences sorted by size, as well as the primer sequences, if desired (step 7). This is a summary of the processes run in the UGSH method; however, steps 2 through 7 are typically performed automatically by the UGSH program and are not apparent to the user.

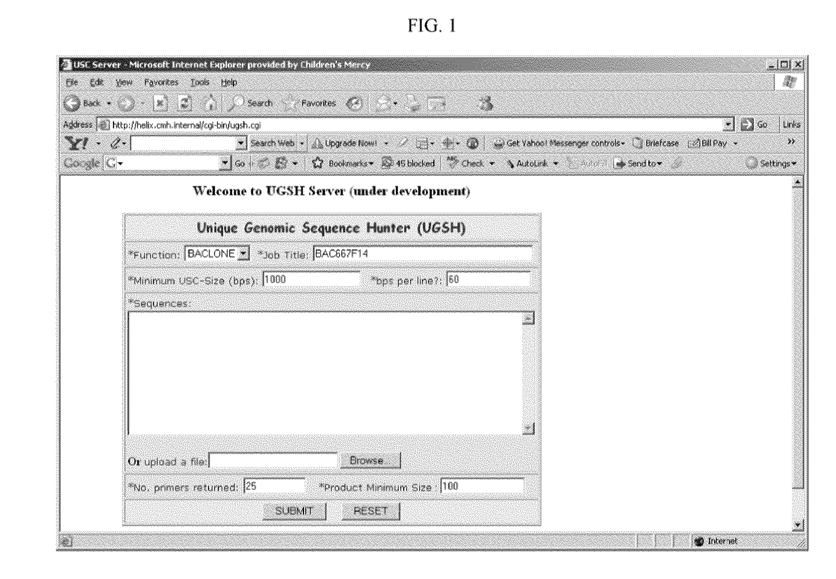

UGSH is preferably implemented as an Internet or web-based application, with the graphical user interface (GUI) provided through one or more Internet browser windows. FIG. 1 is a screen capture of the UGSH input page provided through a web-based interface. A user enters in a job title, minimum size for probe selection, and the number of bases to be displayed per line. The sequence of interest is then either entered in FASTA format into sequence box or uploaded in Genbank file format from NCBI using the browse button by the user. The number of primers to be returned is typically set at 25 as a default parameter, but can be changed by the user. The minimum PCR product size for probes can be changed by the user as well. When all parameters are entered, the user clicks submit to run the UGSH program for unique sequence probe selection.

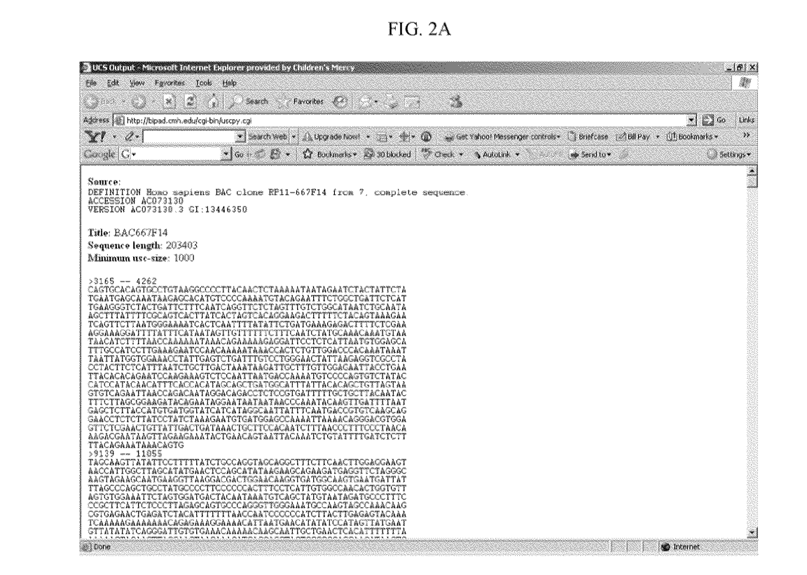

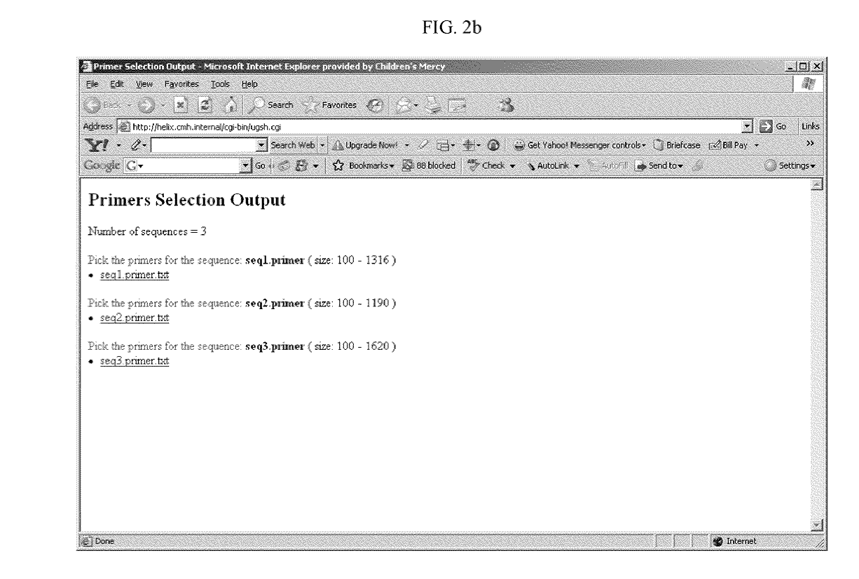

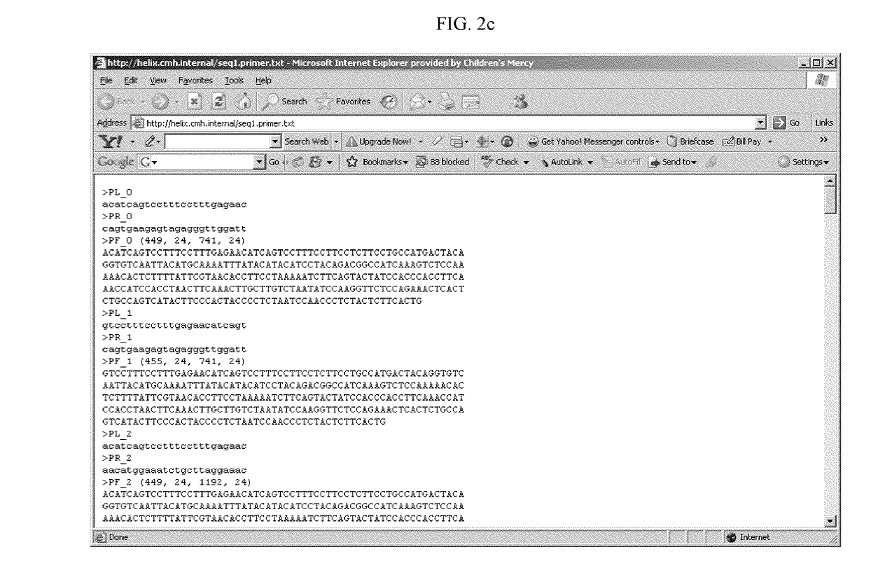

FIG. 2A is a screen shot of a UGSH output page displaying unique sequence regions by position in input sequence. If a Genbank sequence file was uploaded to the UGSH program, the Source lists the definition of the file, accession number of the sequence, version of the sequence (if applicable) and GI number for the sequence, all determined by Genbank. The title of the job, as specified by the user, is displayed as well as the total length of the sequence input by the user. The minimum size allowed for unique sequence probe selection, as specified in the input screen, is shown. The locations of the unique sequence regions are displayed (eg. “>3165-4262”) followed by the actual sequences contained by those coordinates. Primers are displayed after the sequence information (FIG. 2B).

FIG. 2B is a screen capture of an example Primer Selection Output screen from the UGSH program displaying the number of sequences for each unique sequence region. In this example, the sequences are named seq1.primer, seq2.primer, etc, and the size of each unique sequence region used for the primer design is shown in parentheses. The file containing the actual 25 primer sequences, or the number specified by the user in the input screen, is displayed when the text file is opened (FIG. 2C).

FIG. 2C is a screen capture of an example primer sequence file from UGSH displayed in FASTA format. Once the user clicks on the primer sequence file, the primer sequence file is displayed. “PL” indicates the left primer of the unique sequence region and “PR” refers to the right primer. “PF”, for full probe, displays in parentheses the starting position of the left primer, length of left primer, starting position of the right primer, and length of the right primer in relation to the input sequence in parentheses. The region encompassed and including the primers is shown beneath that. Each subsequent primer is shown and numbered 0 to n, where n is the number of primers to be shown specified by the user on the UGSH input screen. The graphical interface (FIG. 1) is used for sequence entry (step 1 or step 1a). After the “submit” button is clicked, the unique sequence probes and primers are displayed (FIGS. 2A, 2B, 2C) which represents the last step of the process (step 7). All other intermediate steps are not apparent (not visible or requiring user interaction) to the UGSH user.

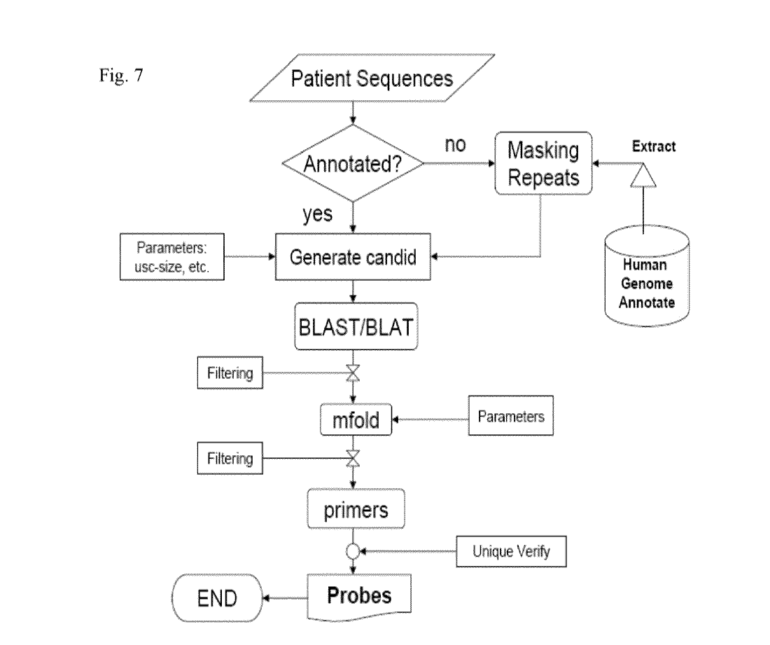

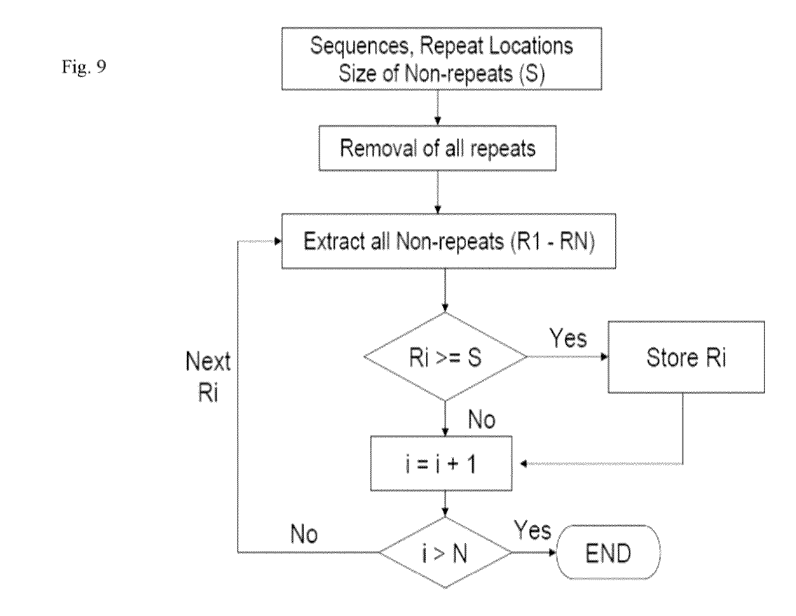

FIG. 7 outlines the following procedure: given a patient sequence or sequences (input), if the sequence or sequences are already annotated (i.e. locations of repeat sequences are known), then candidate unique sequences are directly generated (see FIG. 9), otherwise the repeat locations are determined and the program returns to the next step. The generated candidate sequences are stored in FASTA file format and are run with BLAST or BLAT (default settings) which singles out all those segments that do not satisfy user, third party, or default criteria. The remaining sequences are passed through the Mfold program from which the output sequences are sent to be processed by the Primer3 program. The Primer3 program generates probes. The probes are verified by re-running the BLAT or BLAST program. Each step has filtering thresholds that are detailed elsewhere in this application.

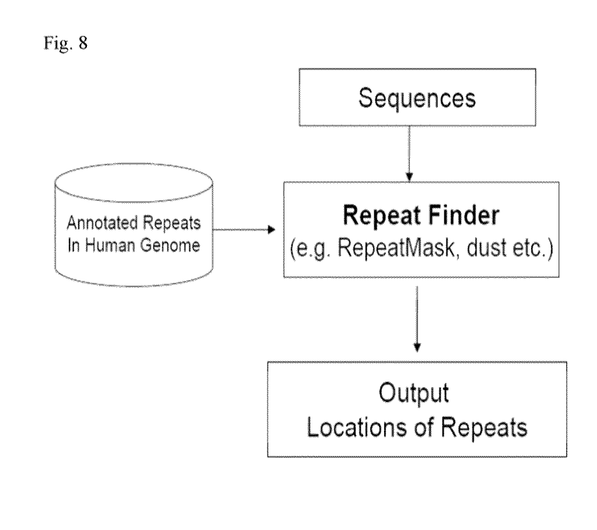

A patient sequence is often retrieved from the NCBI database and thus it is marked with the annotated features (i.e. repeat locations etc.), see FIG. 8. If not annotated, a publicly available repeat finder program such as RepeatMasker or Dust, etc., is used to determine known repetitive sequences within the patient sequence. The output provided by such programs comprises a listing of all the repeat sequences and locations, typically in FASTA format.

As illustrated in FIG. 9, the candidate sequences are generated by removing all the repeats and extracting all the remaining sequences with a size of interest. The output sequences are stored in a formatted file that is consistent with the next program (i.e. FASTA format).

An exemplary embodiment of the UGSH program is presented in pseudocode herein. As presented, the program is organized into modules that interact with one another, and with other programs and data available on the Internet, as the program is used. It is understood that the methods herein are preferably performed by a processor or program within a computer.

```
Main control function
Create Web User Interface {
    Parameters
      Parameters included in preferred embodiment:
```

| | |
|---|---|
| (1) | Job Title (text) |
| (2) | Minimum unique sequence size (integer, 1000 bps) |
| (3) | Number of base pairs per line (integer, default = 60 bps) |
| (4) | Sequences (either a uploaded file or text) |
| (5) | Number of primers returned (integer, default = 25 bps) |
| (6) | Minimum product size (integer, default = 100 bps) |

Optional parameters:

| | |
|---|---|
| (7) | parameters for Mfold (see listing below and/or Mfold website) |
| (8) | parameters for BLAT/BLAST (see listing below and/or BLAT/BLAST |
| | website) |

```
    Options
      Options included in preferred embodiment
```

| | |
|---|---|
| (1) | Processing patient sequences |
| (2) | Generate primers |

Options included in alternative embodiments

| | |
|---|---|
| (3) | Mfold interface (to be added later) |
| (4) | BLAT/BLAST interface (to be added later) |
| (5) | RepeatMasker interface (to be added later) |

Action buttons

| | |
|---|---|
| (1) | Upload |
| (2) | Submit |
| (3) | Reset |
| (4) | Send results by email (to be added in the future |

```
}
If Upload is true {
    UGSH Process
        Performed on sequence provided in uploaded file
Else if Submit is true {
    UGSH Process
        Performed on sequence entered into UGSH Sequence textbox
Else if Reset is true {
    Reset all parameters as defaults
}
}
Else {
    Wait for signal (i.e. click a button)
}
UGSH Process
{
  Input: patient sequences
  Output: probes
  Read Sequences (FASTA format required)
  If Sequences are annotated {
    Extract repeat features (e.g. locations)
    Generate a new file containing non-repetitive sequences
  }
  Else {
    Run a repeat-finding program (e.g. RepeatMasker)
    Extract repeat features
    Generate a new file containing non-repetitive sequences
  }
// The following procedure is a pipeline of modules that
// are typically run sequentially (each module
// running a different program with a set of filtering
// parameters):
  Run BLAT or BLAST with the above generated sequences
  Filtering the output from BLAT or BLAST
  Run Mfold with the above filtered sequences
  Collect those sequences passed through Mfold testing
  Run Primer3 with the above collected sequences
  Collect the output from Primer3
  Run BLAT or BLAST with the Primer3 output sequences
  Output the verified sequences as the probes
}
Repeat-finding
{
  Input: target sequences in a file
  Output: non-repetitive sequences in a file
  Run RepeatMasker with default parameters
  Extract features
  Save non-repetitive sequences in a file
}
Read Sequences
{
  Upload a sequence file
  Parse each line {
    If it is a sequence name {
      Store it the name array
    }
    If it is a DNA sequence {
      Store it in the sequence array
    }
    If the file contains illegal sequences {
      Stop processing and give warning
      Exit program
    }
  }
}
Extract repeat features
{
  Input: annotated target sequences
  Output: non-repetitive sequences in a file
  For each repeat in the repeat annotatiion{
    read the location and repeat length
    remove it until the next repeat occur
    keep the non-repetitive segment in between
    if the segment size >= a specified threshold
    {
      Name and Store it in the file
        Naming convention: Each non-repetitive sequence is named by the target
        sequence name followed by its location range
        Storage format: FASTA sequence format by default
    }
    Else
    {
      Skip it
    }
  }
}
Run BLAT or BLAST
{
  Input: non-repetitive sequences in a file
  Output: unique sequences against human genomic sequence
  Run BLAT or BLAST with default parameters
  Scan the BLAT/BLAST-output {
    If it is unique homologous sequence {
    Store as a candidate sequence to a data file
        }
    Else {
        Do not retain sequence
    }
}
Run Mfold
{
  Input: unique candidate sequences from BLAT/BLAST
  Output: thermodynamically stable sequences in a file
    Optional: pass one or more variables calculated by Mfold pertaining to sequence
    thermodynamics/folding structure to UGSH for presentation to user in UGSH GUI
    window and/or local storage in data file
  Run Mfold with a set of parameters specified
    Parameters provided by UGSH to Mfold (default settings established in Mfold
    program may be used for most parameters)
    Sequence Name
    Sequence
    Folding Constraints
        Force a specific base pair or helix to form
        Prohibit a specific base pair or helix from forming
        Force a string of consecutive bases to pair
        Prohibit a string of consecutive bases from pairing
        Prohibit a string of consecutive bases from pairing with another string
    Specify Linear or Circular Sequences
    Folding Temperature
    Ionic Conditions (i.e., molarity of Na⁺ and Mg⁺⁺)
    Percent Suboptimality
    Window Parameter
    Maximum Distance Between Paired Bases
  Scan the Mfold-output {
    If output indicates that sequence is thermodynamically stable (criteria specified)
    {
        Store as a candidate sequence to a data file
    }
    Else
    {
        Do not retain sequence
    }
  }
}
Run Primer3
{
  Input: stable unique sequences
  Output: genomic probe sequences
  Run Primer3 with a set of parameters specified
    Parameters provided by UGSH to Primer3:
    PRIMER_MAX_END_STABILITY=9.0
    PRIMER_MAX_MISPRIMING=12.00
    PRIMER_PAIR_MAX_MISPRIMING=24.00
    PRIMER_MIN_SIZE=18
    PRIMER_OPT_SIZE=24
    PRIMER_MAX_SIZE=27
    PRIMER_MIN_TM=57.0
    PRIMER_OPT_TM=60.0
    PRIMER_MAX_TM=63.0
    PRIMER_MAX_DIFF_TM=100.0
    PRIMER_MIN_GC=20.0
    PRIMER_MAX_GC=80.0
    PRIMER_SELF_ANY=8.00
    PRIMER_SELF_END=3.00
    PRIMER_NUM_NS_ACCEPTED=0
    PRIMER_MAX_POLY_X=5
    PRIMER_OUTSIDE_PENALTY=0
    PRIMER_FIRST_BASE_INDEX=1
    PRIMER_GC_CLAMP=0
    PRIMER_SALT_CONC=50.0
    PRIMER_DNA_CONC=50.0
    PRIMER_MIN_QUALITY=0
    PRIMER_MIN_END_QUALITY=0
    PRIMER_QUALITY_RANGE_MIN=0
    PRIMER_QUALITY_RANGE_MAX=100
    PRIMER_WT_TM_LT=1.0
    PRIMER_WT_TM_GT=1.0
    PRIMER_WT_SIZE_LT=1.0
    PRIMER_WI_SIZE_GT=1.0
    PRIMER_WT_GC_PERCENT_LT=0.0
    PRIMER_WT_GC_PERCENT_GT=0.0
    PRIMER_WT_COMPL_ANY=0.0
    PRIMER_WT_COMPL_END=0.0
    PRIMER_WT_NUM_NS=0.0
    PRIMER_WT_REP_SIM=0.0
    PRIMER_WT_SEQ_QUAL=0.0
    PRIMER_WT_END_QUAL=0.0
    PRIMER_WT_POS_PENALTY=0.0
    PRIMER_WT_END_STABILITY=0.0
    PRIMER_PAIR_WT_PRODUCT_SIZE_LT=0.0
    PRIMER_PAIR_WT_PRODUCT_SIZE_GT=0.0
    PRIMER_PAIR_WT_PRODUCT_TM_LT=0.0
    PRIMER_PAIR_WT_PRODUCT_TM_GT=0.0
    PRIMER_PAIR_WT_DIFF_TM=0.0
    PRIMER_PAIR_WT_COMPL_ANY=0.0
    PRIMER_PAIR_WT_COMPL_END=0.0
    PRIMER_PAIR_WT_REP_SIM=0.0
    PRIMER_PAIR_WT_PR_PENALTY=1.0
    PRIMER_PAIR_WT_IO_PENALTY=0.0
    PRIMER_INTERNAL_OLIGO_MIN_SIZE=18
    PRIMER_INTERNAL_OLIGO_OPT_SIZE=20
    PRIMER_INTERNAL_OLIGO_MAX_SIZE=27
    PRIMER_INTERNAL_OLIGO_MIN_TM=57.0
    PRIMER_INTERNAL_OLIGO_OPT_TM=60.0
    PRIMER_INTERNAL_OLIGO_MAX_TM=63.0
    PRIMER_INTERNAL_OLIGO_MIN_GC=20.0
    PRIMER_INTERNAL_OLIGO_MAX_GC=80.0
    PRIMER_INTERNAL_OLIGO_MAX_POLY_X=5
    PRIMER_IO_WT_TM_LT=1.0
    PRIMER_IO_WT_TM_GT=1.0
    PRIMER_IO_WT_SIZE_LT=1.0
    PRIMER_IO_WT_SIZE_GT=1.0
    PRIMER_IO_WT_GC_PERCENT_LT=0.0
    PRIMER_IO_WT_GC_PERCENT_GT=0.0
    PRIMER_IO_WT_COMPL_ANY=0.0
    PRIMER_IO_WT_NUM_NS=0.0
    PRIMER_IO_WT_REP_SIM=0.0
    PRIMER_IO_WT_SEQ_QUAL=0.0
  Collect the output from Primer3
  Run BLAT or BLAST with the Primer3 output sequences
  Output the verified sequences as the probes
}
Note: Data is passed between UGSH and utility programs (Mfold, BLAT/BLAST, Primer3, etc.)
via text file or parameter options provided by one of the programs. These parameters can be
received via web interface, predefined in a file, or contained in the UGSH program (i.e. Perl)
scripts if treated as constants.
```

### DEFINITIONS

Unless defined otherwise, all technical and scientific terms used herein have the same meaning as is commonly understood by one of skill in the art to which this invention belongs at the time of filing. If a definition provided below is different from or broader than a “definition” provided elsewhere in this application, the definition below will control.

“Nucleic acid” and “nucleic acids” herein generally refer to large, chain-like molecules that contain phosphate groups, sugar groups, and purine and pyrimidine bases. Two general types are ribonucleic acid (RNA) and deoxyribonucleic acid (DNA). The terms are inclusive of hybrids of DNA and RNA (DNA/RNA) and ribosomal DNA (rDNA). The bases naturally involved are adenine, guanine, cytosine, and thymine (uracil in RNA). Artificial bases also exist, e.g. inosine, and may be substitute to create a nucleic acid probe. The skilled artisan will be familiar with these artificial bases and their utility.

“Low copy nucleic acid segments” and “low copy segments” are synonymous terms referring to nucleic acid sequences of varying length that are “unique”, i.e. non-repetitive, nearly unique, or so infrequent in a normal chromosome or genome to not be classified as repetitive by the skilled artisan.

“Repetitive DNA”, “repeat sequences” and variants thereof refer to DNA sequences that are repeated in the genome. One class termed highly repetitive DNA consists of short sequences, 5-100 nucleotides, repeated thousands of times in a single stretch and includes satellite DNA. Another class termed moderately repetitive DNA consists of longer sequences, about 150-300 nucleotides, dispersed evenly throughout the genome, and includes what are called Alu sequences and transposons.

“Sequence” and “segment” are interchangeable terms and refer to a fragment of nucleic acids of variable length.

“Hybridization” as used herein generally refers the pairing (tight physical bonding) of two complementary single strands of RNA and/or DNA to give a double-stranded molecule. Hybridization techniques are inclusive of both solid support technologies, such as microarrays, southern blot analysis, and quantitative microsphere hybridization, that separate the target nucleic acids from their biological structure and of cell or chromosome-based technologies that do not separate the target nucleic acid from their biological structure, e.g. cell, tissue, cell nucleus, chromosome, or other morphologically recognizable structure.

“PCR” means polymerase chain reaction.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

This invention has been tested using quantitative microsphere hybridization (QMH) and fluorescent in situ hybridization (FISH).

### QMH Analysis

Unique sequence probes (100 bp) specific to HOXB1 (chr17: 43964261-43964360) (all references to coordinates in this application refer to the March 2006 UCSC Genome Build) and the DiGeorge (DG) Critical Region (chr22: 19079557-19079656) were designed using the UGSH method and synthesized from normal control genomic DNA by PCR (Promega). The forward primer for each probe was synthesized with a 5′ six carbon linker followed by an amine group (Invitrogen) and these probes were attached to spectrally distinct polystyrene carboxylated microspheres (Luminex) via a modified carbodiimide coupling reaction (Newkirk et al. 2006). Target DNA was prepared for hybridization by incorporation of biotin-16-dUTP using whole genome amplification for two different DiGeorge patient genomic DNA samples as well as one normal control sample. Biotinylated genomic DNA was sheared to an average size of 1 kb and the DiGeorge probe and HOXB1 probe were hybridized in a multiplex reaction. Samples were analyzed by dual-laser flow cytometry (Luminex) and the mean fluorescence intensity (MFI) ratios for each probe obtained. Data for the DiGeorge patients (DG-1, DG-2) and normal control sample are displayed below.

TABLE 1

| | Probes | | | |
|---|---|---|---|---|
| Samples | HOXB1 MFI | MFI ratio | DG MFI | DG MFI ratio |
| DG-1 | 123 | 1 | 65 | 0.53 |
| DG-2 | 109 | 1 | 57 | 0.52 |
| Normal | 173 | 1 | 171 | 0.99 |

The MFI value for the HOXB1 probe was 123 and the MFI value for the DiGeorge probe was 65. This constitutes an MFI ratio of ˜0.5 which indicates the DiGeorge probe is present in only one copy as compared to the HOXB1 probe present in two copies, which is reflective of the actual genotype of the DiGeorge patient DNA. This example illustrates that UGSH successfully identified unique sequence regions since an MFI ratio greater than ˜0.5 would indicate that the DiGeorge probe hybridized to other genomic regions and was thus not composed solely of unique sequence. Examples of QMH probes not effectively designed specific to unique sequence regions (that is using the prior art methods) yielded MFI ratios not ˜0.5 in patients with deleted genomic regions and were presented in Newkirk et al., 2006 (Human Mutation).

### FISH Analysis

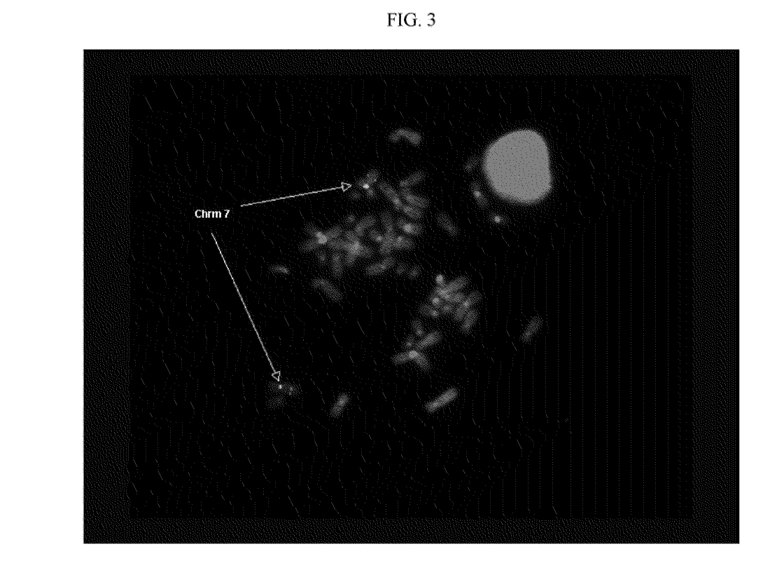

Additionally, this invention was used to design unique sequence probes for FISH analysis. Genomic sequence specific to BAC RP11-677F14 (203 kb; 7q31) was uploaded into UGSH (FIG. 1), the program was executed, and unique sequence probes were displayed (FIG. 2). One probe (chr7: 115367602-115371201) and corresponding primer sequences were selected from the UGSH output and synthesized the primers (Invitrogen). The specific genomic region was amplified by PCR (Promega). Standard methods for direct probe labeling (Mirus, Inc.) were used and the probe was hybridized to normal human control chromosomes (metaphase and interphase) using FISH. The single unique sequence probe produced very bright and distinct hybridization signals (FIG. 3) indicating no cross-hybridization to other genomic regions, thus verifying its unique sequence design.

FIG. 3 is a photograph taken from a FISH experiment using a unique sequence probe from BAC RP11-677F14 on chromosome 7 designed using the UGSH method. A Cen7 probe (green; Vysis) specific to the centromere of chromosome 7 was hybridized to a normal human metaphase chromosomal spread as a control probe. The BAC RP11-677F14 probe (red) was concurrently hybridized. This experiment shows no non-specific binding of the BAC RP11-677F14 probe to any other chromosomal regions, thus proving this probe is composed of unique DNA sequences only and validating the UGSH method.

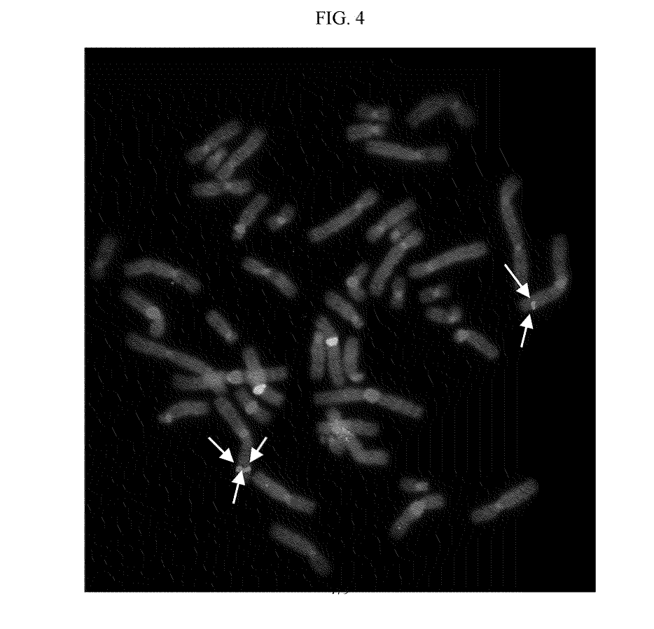

This technology has been extended to create unique sequence probe cocktails which are simply five or more unique sequence probes combined in one FISH experiment. FIG. 4 illustrates results obtained from using five unique sequence probes specific to chromosome 3, which were designed using the UGSH method. Each probe was PCR amplified and direct labeled (red; Mirus, Inc.), then combined and co-hybridized with a control probe (Cen7, green; Vysis) onto normal human metaphase chromosomes. The signal intensity for hybridization in this FISH experiment was much greater for the unique sequence probe cocktail, as compared to the single unique sequence probe (FIG. 3), and exhibited very little background fluorescence, allowing for faster and easier localization.

Such probe cocktails would be ideal for commercial FISH probes since they are comparable in signal to current FISH probes which are much greater in size (˜300 kb), however unique sequence probe cocktails would allow for a more accurate diagnosis of a chromosomal abnormality due to their significantly smaller size (˜10 kb total). These experiments illustrate the utility of this novel method for use in designing unique sequence FISH probes.

The unique sequence probes designed by UGSH were compared to other methods available for single copy probe generation in the prior art (e.g. the '097 and '997 patents). In one FISH experiment, a probe not designed using the UGSH method, but rather designed using a method presented in the '097 and '997 patents was used. Repeats in a DNA sequence specific to chromosome 9 were masked by homology searches with well known repeat families and classes (the '097 and '997 patents) and primers were designed to one resulting purportedly “single copy” region (ABL1 probe 16-1, Knoll and Rogan, 2003).

Results from the FISH experiment show hybridization of the probe (red) to numerous chromosomal locations indicating this sequence is homologous to more than one chromosomal region and thus not composed of purely unique sequence. A control probe specific to the centromere of chromosome 9 (CEP9, Vysis) was co-hybridized during the FISH experiment. Further analysis of the ABL1 probe sequence itself revealed that 61.98% of the probe sequence was composed of repetitive elements, including Alu, LINE1, and LINE2. Because these elements are slightly divergent from the ancestral repetitive sequence for each element, repeat masking was not sufficient to identify these sequences.

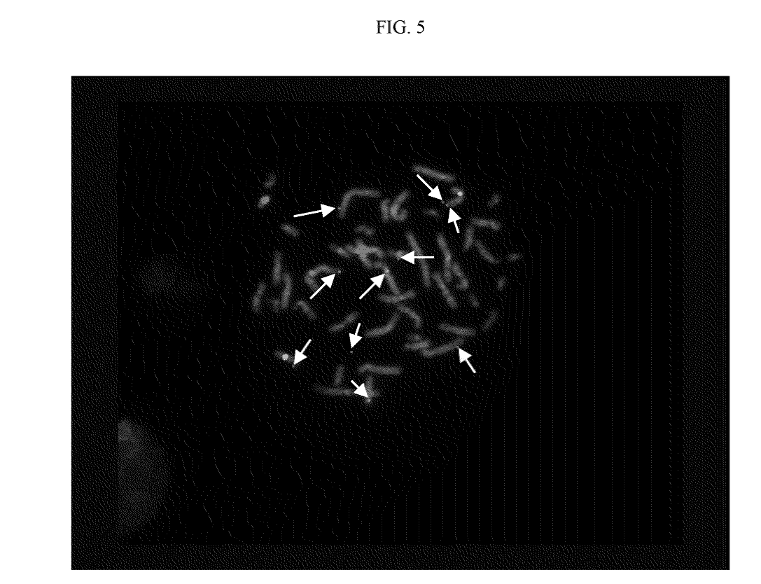

When this sequence was analyzed by BLAT, greater than 150 matches were identified across the genome with the majority of BLAT scores ranging from 215 to 100. In contrast, a preferred cut-off BLAT score for the UGSH method is 25 to allow for very strict selection of unique sequence probes. The outcome of this more stringent cut-off value for unique sequence probe selection is evident when FIGS. 3 and 4 are compared with FIG. 5.

FIG. 5 is a photograph taken from a FISH experiment using a probe not designed using the UGSH method, but a method presented in the '097 and '997 patents. Repeats in a DNA sequence specific to chromosome 9 were masked by homology searches with well known repeat families and classes (the '097 and '997 patents) and primers were designed to one resulting “single copy” region. Results from the FISH experiment show hybridization of the probe (red) to numerous chromosomal locations indicating this sequence is homologous to more than one chromosomal region and thus not composed of purely unique sequence. A control probe specific to the centromere of chromosome 9 (CEP9, Vysis) was co-hybridized during the FISH experiment.

If a researcher's particular experiment called for less strict parameters for the identification of such sequences or less stringent thermodynamic boundaries, there is an option for the user to change these variables. This would result in a greater number of sequences being identified; however the performance of such sequences in a genomic hybridization experiment might be compromised.

Further uses of the UGSH method include the generation of probes for any genomic hybridization experiment. UGSH can identify unique sequence probes (60-70 bases) for microarray and arrayCGH experiments. Primer sequences would not be necessary for these applications due to the short length of probes, however UGSH would display the necessary unique sequence regions. Other applications for the UGSH method include but are not limited to Southern and Northern blot analysis, in situ hybridization, multiplex ligation-dependent probe amplification (MLPA), and multiplex amplifiable probe hybridization (MAPH).

### Example 2

This Example provides a number of probes that were developed using the methods of the present invention. Each of the probes can be used individually, or in combination with at least one other probe in order to assess the risk of uterine cervical cancer. When these probes hybridize with the target nucleic acid sequence, risk of developing uterine cervical cancer is reduced as the sequence of interest is known to be present. However, if hybridization does not occur, the sequence of interest is deleted, or has mutated to a point that prevents hybridization. Such a situation indicates that the individual is at an increased risk level for developing uterine cervical cancer. In some forms of this aspect of the invention, a single probe selected from the group consisting of SEQ ID NOs. 1-31, is used in the hybridization assay. Again, an absence of hybridization leads to a conclusion that the individual has a higher risk of developing uterine cervical cancer than the general population, as well as in comparison to individuals whose genome contains the sequence of interest. In other preferred forms, a combination of probes is used. Even more preferably, the method will include at least 2 or more probes selected from the group consisting of SEQ ID NOs. 1-25, or SEQ ID NOs. 26-31. The probes from SEQ ID NOs. 1-25 are from chromosome 3 (3q26), and the probes from SEQ ID NOs. 26-31 are from chromosome 7. In some preferred forms, probe cocktails containing a plurality of probes are used. As the sequence and location of hybridization for each probe is known, the hybridization (or lack thereof) of any one probe will provide a wealth of information related to the intactness, or variation in comparison to a sequence without variation, all of which may aid in the detection and risk assessment of individuals for uterine cervical cancer.

Similarly, SEQ ID NOs. 32-43 also relate to genetic markers for uterine cervical cancer. Absence of hybridization of any one or more of SEQ ID NOs. 32, 35, 38, and 41, is associated with an increased risk of developing uterine cervical cancer, while hybridization of any one of these probes is indicative of a normal genetic sequence and a non-elevated risk of developing uterine cervical cancer. SEQ ID NOs. 33 and 34, are the forward and reverse primers, respectively, for SEQ ID NO. 32, SEQ ID NOs. 36 and 37, are the forward and reverse primers, respectively, for SEQ ID NO. 35, SEQ ID NOs. 39 and 40, are the forward and reverse primers, respectively, for SEQ ID NO. 38, and SEQ ID NOs. 42 and 43, are the forward and reverse primers, respectively, for SEQ ID NO. 41. As with SEQ ID NOs. 1-31, the probes of SEQ ID Nos 32, 35, 38, and 41 may be used individually, or in combination with one another, or even in combination with any of SEQ ID NOs. 1-31. Table 2 provides a listing of coordinates for each of these probes (according to the March 2006 UCSC Genome Build).

TABLE 2

| | | | | |
|---|---|---|---|---|
| | Start | End | Probe | SEQ ID |
| Probe name | Coordinate* | Coord | size | NO. |

Chromosome 3q26 Probe cocktail:
All probes pooled together in one reaction

| | | | | |
|---|---|---|---|---|
| RP11-641D5-8 | 170468591 | 170470501 | 1910 | 1 |
| RP11-641D5-7 | 170472622 | 170474906 | 2284 | 2 |
| RP11-641D5-6 | 170491470 | 170494165 | 2695 | 3 |
| RP11-641D5-5 | 170495466 | 170498705 | 3239 | 4 |
| RP11-641D5-4 | 170504182 | 170507036 | 2854 | 5 |
| RP11-641D5-3 | 170513776 | 170515778 | 2002 | 6 |
| RP11-641D5-2 | 170551404 | 170553206 | 1802 | 7 |
| RP11-641D5-1 | 170564835 | 170568441 | 3606 | 8 |
| RP11-3K16-5 | 170571082 | 170573293 | 2211 | 9 |
| RP11-3K16-4 | 170616435 | 170618896 | 2461 | 10 |
| RP11-3K16-3 | 170633935 | 170636538 | 2603 | 11 |
| RP11-3K16-1 | 170702962 | 170704398 | 1436 | 12 |
| RP11-816J6-1 | 170782158 | 170783927 | 1769 | 13 |
| RP11-816J6-2 | 170811261 | 170813516 | 2255 | 14 |
| RP11-362K14-3 | 170821049 | 170822942 | 1893 | 15 |
| RP11-362K14-2 | 170824210 | 170827979 | 3769 | 16 |
| RP11-362K14-1 | 170860403 | 170861821 | 1418 | 17 |
| RP11-379K17-5 | 171017787 | 171020006 | 2219 | 18 |
| RP11-379K17-4 | 171031245 | 171034304 | 3059 | 19 |
| RP11-379K17-3 | 171131084 | 171135002 | 3918 | 20 |
| RP11-379K17-2 | 171135323 | 171138745 | 3422 | 21 |
| RP11-379K17-1 | 171138881 | 171142114 | 3233 | 22 |
| RP13-81O8-1 | 171140257 | 171142304 | 2047 | 23 |
| RP13-81O8-2 | 171166207 | 171168262 | 2055 | 24 |
| RP13-81O8-3 | 171209493 | 171210861 | 1368 | 25 |

Chromosome 7 probe cocktail:
all probes pooled together in one reaction

| | | | | |
|---|---|---|---|---|
| BAC667F14-1 | 115561346 | 115564397 | 3051 | 26 |
| BAC667F14-2 | 115597264 | 115601247 | 3984 | 27 |
| BAC667F14-3 | 115667956 | 115669681 | 1950 | 28 |
| BAC667F14-4 | 115676311 | 115678653 | 2343 | 29 |
| BAC667F14-5 | 115685858 | 115688020 | 2162 | 30 |
| BAC667F14-6 | 115698372 | 115700626 | 2254 | 31 |

*March 2006 UCSC Genome Build

Finally probes developed in accordance with the present invention are particularly well suited for use in quantum microsphere hybridization assays. Preferred probes include those provided herein as SEQ ID NOs. 44-57. Each one of these probes is used individually to detect the presence of the pathogen from which it is derived. SEQ ID NO. 44 is from the Mycoplasma FRX A Gene (genus specific). Specifically, hybridization of SEQ ID NO. 45 indicates the presence of M. Fermentans, hybridization of SEQ ID NO. 46 indicates the presence of M. mollicutes, hybridization of SEQ ID NO. 47 indicates the presence of M. hominis, hybridization of SEQ ID NO. 48 indicates the presence of M. hyorhinis, hybridization of SEQ ID NO. 49 indicates the presence of M. arginini, hybridization of SEQ ID NO. 50 indicates the presence of M. orale, hybridization of SEQ ID NO. 51 indicates the presence of Acheoplasma laidlawii, hybridization of SEQ ID NO. 52 indicates the presence of M. salivarium, hybridization of SEQ ID NO. 53 indicates the presence of M. pulmonis, hybridization of SEQ ID NO. 54 indicates the presence of M. pneumoniae, hybridization of SEQ ID NO. 55 indicates the presence of M. pirum, hybridization of SEQ ID NO. 56 indicates the presence of M. capricolom and hybridization of SEQ ID NO. 57 indicates the presence of Helicobacter pylori.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the following claims.

### REFERENCES

The entire teachings and content of the following references are specifically incorporated herein by reference:
U.S. Pat. No. 7,014,997, “Chromosome structural abnormality localization with single copy probes,” Rogan and Knoll, 2006.
U.S. Pat. No. 7,013,221, “Iterative probe design and detailed expression profiling with flexible in-situ synthesis arrays,” Friend et al., 2006
U.S. Pat. No. 7,115,709, “Methods of staining target chromosomal DNA employing high complexity nucleic acid probes,” Gray et al., 2006
U.S. Pat. No. 6,828,097 “Single copy genomic hybridization probes and method of generating the same,” Rogan and Knoll, 2004
U.S. Pat. No. 6,242,184, “In-situ hybridization of single-copy and multiple-copy nucleic acid sequences,” Singer et al., 2001
Andresson, R, Reppo, E, Kaplinkski, L, Remm, M. GENOEMASKER package for designing unique genomic PCR primers, BMC Bioinformatics, 2006, 27(7): 172.
Knoll, J H M and Rogan, P K. Sequence-based, In Situ detection of chromosomal abnormalities at high resolution, American Journal of Medical Genetics. 2003, 121A:245-257.
Miura, F, Uematsu, C, Sakaki, Y, Ito, T. A novel strategy to design highly specific PCR primers based on the stability and uniqueness of 3′-end subsequences. Bioinformatics, 2005, 21 (24):4363-70.
Newkirk H, Knoll J F M, Rogan P (2005) Distortion of quantitative genomic and expression hybridization by Cot-1 DNA: mitigation of this effect. Nucleic Acids Research 33:e191.
Newkirk H, Miralles M, Rogan P, Knoll J H M (2006) Determination of genomic copy number with quantitative microsphere hybridization. Human Mutation 27:376-386.
Rogan, P K, Cazcarro, P M, Knoll, J H. Sequence-based design of single-copy genomic DNA probes for fluorescence in situ hybridization. Genome Research, 2001, 11(6):1086-94.
Rozen S, Skaletsky H. J: Primer3 on the WWW for general users and for biologist programmers. In: Krawetz S, Misener S (eds) Bioinformatics Methods and Protocols: Methods in Molecular Biology. Humana Press, Totowa, N.J., 365-386 (2000).
Tatusova, T A and Madden, T L. Blast 2 sequences—a new tool for comparing protein and nucleotide sequences, FEMS Microbiol Lett., 1999, 174:247-250.
Zuker M: Mfold web server for nucleic acid folding and hybridization prediction.
Nucleic Acids Res 31: 3406-3415 (2003).
RepeatMasker: Smit, A F A, Hubley, R, Green, P. unpublished. Current Version: open-3.1.6
BLAT: UCSC Genome Browser website on the world wide web, the address of which reads in pertinent part “genome.ucsc.edu”.

What is claimed is:

1. A method of identifying a low copy nucleic acid segment comprising two or more of the following steps:
(a) removing highly and moderately repetitive sequences from a genomic region of interest and displaying non-repetitive genomic segments;
(b) searching it non-repetitive genomic segment for homology to genomic regions other than the region of interest and discarding all segments that are homologous to a genomic region not of interest;
(c) identifying possible secondary structure motifs in a non-repetitive genomic segment; and
(d) designing a probe from a non-repetitive segment identified b) at least one of steps a, b, or c and analyzing the probe for uniqueness as compared to the genomic region of interest and genomic regions not of interest.

2. The method of claim 1 comprising at least 3 of steps a-d.

3. The method of claim 1, wherein said non-repetitive genomic segments of step a have a size greater than 1 kb.

4. The method of claim 1, wherein step c is performed by thermodynamic analysis.

5. The method of claim 1, further comprising the step of designing PCR primers for genomic segments resulting from the performed method.

6. The method of claim 5, further comprising the step of ensuring said PCR primers contain only unique sequence.

7. A method of selecting probes used for hybridization experiments comprising the steps of:
(a) removing repetitive sequences from a sequence of interest to provide a sequence segment;
(b) comparing each said sequence segment to genomic regions other than the region containing the sequence of interest and discarding all said segments that match elsewhere in said genomic regions and retaining the remaining unique sequences;
(c) evaluating said unique sequences for possible secondary structure motifs; and
(d) selecting probes based on said unique sequences that do not have possible secondary structure motifs.

8. The method of claim 7, further comprising the step of designing PCR primers for said probes.

9. The method of claim 8, further comprising the step of ensuring said PCR primers do not match elsewhere in the genome.

10. The method of claim 7, wherein step (c) is performed using thermodynamic analysis.

11. The method of claim 10, wherein said thermodynamic analysis is based on Gibb's Free Energy Equation wherein the Gibb's Free Energy is between 0 and 50.

12. The method of claim 11, wherein ΔH<−1000, ΔS<−3500, and Tm≧37 C in the Gibb's Free Energy Equation.

13. The method of claim 12, wherein Tm is ≧42 C.

14. The method of claim 12, wherein Tm is ≧60 C.

15. A nucleic acid sequence selected from the group consisting of SEQ. ID Nos. 1-57.


SEQUENCE LISTING (57 sequences)

SEQ ID NO: 1 - DNA, 1911 bp, Homo sapiens
ctttctccatctacaatgtcctttaccaagttgaaactaaatcatctgggatggaaaatctttgatagcttttcataccaaccttgtgaaaaaagatgtatgtctaagtttatggcgacaaacagtgttattatctctcctcctctaatatttaaccaacctaaacactcaggggaaaaatgttatctgaaatccaaccactttatatacattcacttggtcttctttggacaagtcaaccagctttttctcacaagtatctcaaatacttgtttggaaagcttttctcaagtgggtatgcaaacaggaggagaactgttctttagcagtcccttcttataagcctccaatgatgctatcttcttgtactttagttaatgaaaagataaatatgatctgtgctgtgcccagctgtgttgttcttgtactgaggaattcacaaaaggaaaattttacatcatacttttatcagtgaacaagcttgctaaagcaaatagatttcagcctcctgagtttgcttcaataaaagattaaacgctaatagtgcaacttaccgcactcaacagatttatgaataatcaaagtctggagaaaggccaggccatttaagaacattacctaccaaacagatgccagtagtatgtagacgtcttccctatagccaagcagctgtatttcctatcgcacaactcaatattgactgcataattggcctttacaaaacatttattaaaatgcaccatttgtcacagacccagtgtttgtttaagagtttcaggtggggagagggaattgaggggtgggcggaagacacacagtttctaattactaagcggatttaattggtaaacagcagcaagtccagcctatcctggtaagatgaaaggtctgaattttcaaggagcaaggagttctttaagaaataaagagaataagcaccttatgagaactagtttttgaataccactttgcatctgtgaatggcattcctctcggtgaacacaaagcccatttccatgtgtttaccttcgtgttctcatgctattcctaggtggtcagcctgccattatgttgctgctctccttttctagattaatacactgaggcatggagacttttttgaatgctctatctacccaaaggagcatattaagttaatgattaagtgagggctgaagtcagcgtgctaagtttctaactctttgaaaaggattgcttatttctccatcctgtatctgtatacagctttaaaaagaaaaaaaaaaaatcagttgactgattccttgtaatagaaaccacctatttaaaaaaatctccctgctaacaaattccaaatacatgtggacaaaacgctttaattagaaggcactgagtttagttgcaatgaactcaaaacgcactcagggtatgactcaagaatgaaaagtatcactttattttgtaggtttgccatggcagaaaatgcaactggattccagttaatatcttgccaaattaaaacatgcttcttaatttgttggcctcctgctcaataaaaatcctagtcatgctctctgtaataggaattcctcactaaacctcacttaatgctagaaaaatgagagtatcatttatctctggcacccaagtatgtcttcagcaggtgcttgaatcatttaagatttctctaacaccaaaagctaattattttagttctttggtgctatttttaattacataaacacctttggtttcttcaattttattcaaactttaaaagagagtaggtttgtgttatggcaataattattcattaagtggaaaatagaaatatttttgttcaaattttagctttctaacagctgcatacgtatataaaggtgtgtgtgtgtgtgtgtgtgtgtaggtatttcatactcacttggcccaaag

SEQ ID NO: 2 - DNA, 2285 bp, Homo sapiens
tgcacttagtcttggaagattaaccctttggcagggtgaattcacactgttaaacgcaaaggagtctctcctgagactcatgaaagtgccttagttcccagagtaggacccagagactactggagatctaacatgattcatgggagtcaaagagaagtggctaacatgggagttggggagagacataggctctggaaggaattgaatcacagattcctggagctacagaaaaccttagcagtcaccttagccctctcagtgcaccacacaactggttagtgagacaaactcaatgtagctttctttacattgtccccagaatgttttgaggtaaaagaaataggggcagaaggaacctggtcatagaaaaaaaattatatattattaaagaaacataatagcaaatgaggacaatatatgtaaagataatctttaaaacgttttctgacatggtcctggtatacattttaaattacatctgaatgctgaggagaccatcttagaagctggtgtggatgacgacttcccaataagaatgttaagaaagggagaaagcaaatcatttacacacctggccggcccctgtgaccttcaaaagtcactggctgcacattcagttgcacatacacttttgaaaaacttctctttgtctggcaatgtcattccccttaccccatccctcacacttttcaaatagaaagaaaagggtcatagggtgggcactggctgggtgcaggaggtctcgacctcaccacatctggtgagggcagaggaggggaggcagggaggactgcgggaaagagctggagcaagggtgagaggacagcaagctggccaaggagaaacacctcatccccttaactcctgcttgaagcatctacctcctccaaacacagaaatatgaaaaacaggtagtcagctacagaataggaaattgagatactttccaaggattcaagaggaagaggaatgcatgcatgagttggtcgtatttcagctgcccacatgccgttttatgtgggcacttagaacacattttctataacatccctttgccacaaataaggcatcagaacatttgttacaaagcaagggtatacaaaatacactatatgattttgtatatccaccaccacctgcatactcaaaatcacagagatagattaaaaaggatatagttcaaaatgttaaagccatatatgctgtgtaggagcactttaccaaattacctttgttgcagtcgctgaagttctcatcacaaagagtaaaaaccatcatgcttccacatagaaataagcaaattgcatcagtgaaatataaccaggaagagaaaaggaattcaacctcccccatcaacactaaaactagtcaataattatctaagtggtatcagttatgttttctgcgcatcctggttggcctctaagtaaagagaaggagccggagagaagggcttgggaatgctacggtttctctatagcagttttatcttaggcggtgtgtgtgaagactagggtgtatgcagcgagtaatctgcatgtttactgcgtcaacattctacccacttctgggtagaatcgttgctcatcctaatcacattcaaaggccccatagggcagcaagaagttcctacttactggcaatttagctcttggaaacccttgaagaggatggttttgtccctgtgtgctatcatggctgtgtggaggccactaagccagctcacagctaaaattcaacaggcaggaatgactcacacctctccaaagcttttttttttttttttttttgagatggagtatcattctgttgcccaggctggagtgcagtggtatgatctcagctcactgcaacctctgcctcccgggttcaagtgagtctcctgcctcagcctcccaagtagctgggattacaggcacatgccaccacgcccagctaatttttgcatttttagtagagacagggtttcaccatgttggccaggctggtctcaaactcctgacctcaggtgatctgccctcctcgacctcacaaagtgctgggattacaggcatgaggcactgtgcctggcctccaaagcttcctaaaaatgagtgaggagtactgccaatttaaaagtctgcctgatattcaatagaggaaagatacactttcaataaacattcacttgttttacgtgataatgtaaacttgataatgagaataagaaatctaataaggccattctgtaatgtatatcatgaactaagtatattttcctatttcttagcactacactgttttct

SEQ ID NO: 3 - DNA, 2696 bp, Homo sapiens
ttttcaacagttaccagccacctgcctactaccactaccacctttgtggcctgggaatctatctctgctgaactccacttattaaaaggaaatccttctatatactttacctaaatcttgcatccttcagtcgaagctcatttcatattattcaaactttggtgaacagtagtccaacatcatcctccttattggaaaaatccattatttgaaaatagtcaataaatcaccctttatccttcatttcccccacttgaataagcccagctccctagggctttctccaaagtatgcagcctccaactcactatcaaggaagaactgagttcctgggaaaattagtcatatattataactaattgtaacatgacatattatagaagagtataataattattaattttaaacagtaaatataattgccctaaaacttggcttatactttcctctaaattcctcaggaaattctcaaatttctcctttacctcagcctacttcagaaatgctcaatcctctctgtcagtgtgggagatcagtttatatttaggagaagtgggcaaatgtctggtatggaacacatcaaaactaggaaaatctgttccctcataaactaagttaaaatttctaaaactaaaagacaagttaattaacccattggatatagtcattgttatagcatctattattgaaagtgggctctatttcaagcctcagatgtatagaacattctgaaatgtttttctaggcagtgcatacatgctgatccaataaaactttcttttgctttgtttagtatggagatagctactctgcatgtttcacatcagtatttgattctgtgtctaaatttgagtatcatcttgaatactaggtattctcctctgtagaatctgatcatgtgaataataccacagggagctgcatacttcaagcaaaaactcaagagctgatatttgtttcaacagattcacattttaaaatttattgtctaccatttgtgtatgaggcatgaaacatgacccttatactggaccacaaatgattatgcaactagtttggcgttttgtattttaaagagattggggctcatgctggtaatcccagcatttttttgggaggccgaggcaggtggatcacttgaggtcaggagtttgagagcagcctggccaacatggtgaaaccctgtctctactaaaaattcgaaaattagctgggcatggtggagtgcatctgtaatcccagctactcgggccagagaatcatttgaacccaggaggcagggtagcagtgagctgagatcgcacaaccacaccccagcctgggtgacagagtgagatgccatctcaaaataaaataaaataaagataatcataaacataaatgaaaagattgaactgatagatgatgaagcaagatctattcactgtgcaaggatgaaaggcccctacagaatcccttcctgaaactcaggattgattgtttcaaccctgtgatattctgctttctgaacaagatgccagaattctactgtgagctctgtgcaggctaatttaggagatgcaagatatctgttatctcccagtgatctctctgagccacaaggtcagattcaactgaatattaaggagggccctgaaaaggctcacagggccactttgagaaccataagggtgaagtgcatgacagctggttatgggaaggggagggtgaattctggtggtaccatctctacccagtgaccattcttgtcatcagggtttcctaaatcagcctaataggagactgaagatctgaattcgggtattgcagaatgtttgcagcctcccagggaatggaaaaagatctggatcaaattatattcaatttagaggcatgtggggcattctaatgatttccattgcccctgtggtcatcttgggtggtggagctcattaaggctcaatcacaaaaattagggctcagaggatgtcagacctaaaaaaggacccaatgtggaccacctaagctcagtgagatgaagtgacttatccaagacctcagctccaggccatcttataactaataataggtacctccatgactagcaaaggggtggctgacatttgtcttttaaatacctatcgcaggaggagtcagctagaacaaatcaacagagcatgccgagagtctgagagaaagttatctggcatgagcagtgctagttccagccaccatcatgactcttggaattccagagtaggttcctaactcaccttcatggtttcacgctcaccccaccgtctattcaccacacagccgtcagggtcttctcctggcttcccatcacattcagtccaaactcctcaccatggcctttgaggcccttcctctctgacctcactgctcctctctcattcattcgcactagcctcgtgtgtttggctttctggttctttgaaacatcaagttggtttcttcttcggccgccttggccttgctattctcttaggctgaaatgctttttctgaagctagccccatggctcactgttattcctttcaggtttctgttccagtgtccctgctcagaggggctacacctgacccctgctctaagtaccaaaccttctgtgtgctacaattccccctttactctttatccccttcctcatcttcacttttcttgatagcagttattattaccaaacattatatagt

SEQ ID NO: 4 - DNA, 3240 bp, Homo sapiens
tctagaagattatcttttctattgcttagttttctttatttaaaaaaaaatgttttctatcttaatgttacgtaaaatatgtgcattataattgagatggggattttcaaactgtcttcctgaggttcctcagagatcatcagaggaaagatatgagcacagacctttggctttgaacctcctgtcccactttcaccataggatctaggttgacttttatctattttatatattgggtttctgagtaagatattgtttaaagaaagatttcctattgcttttatttaaaagggttaaaaaaagttaaattacaataacacgtgactcgaaggatttgccaccacctggtggcagcatacccaaatttcaatgtacctaaatttttaaaagggttgagttgaatctgctattctaaaattataaagtggtatcagaaatgtctcagatgatgatgtataattatgaaaaaacattaaaaagtacaatatcactttcaaaatacaatatagtatttcaaagttgagatttttataaatgaattaaattactttattgtagggcatagtacttgtagaaaacatttccattatgcctttaaaacaacattgcaagaaggtaaaataaactagtagcattctctcaaattctttttatgtttgaacatcaatgttacctacctatcctcaaatatctggggaaaacttcatttcttcttcttctccttaaagtcaacataaatactcagtttctgggtaaattgtaccagggacattgcccgtgctgctctctacctccaccaaaacaaaacaaagcaaagacaccttttcacagaactttagaaatgactgcacaaagagaataaaaaacaagtattacaatcacaagtatattcatggacaagtggaggaagaaagtgaggttttgtttgatggtttgtttgcttgtttgttttcctttttcaggcctttcagctctcatacaacagtaacatattggttgaaccaactgaccttgctctggcctgattcaaccatagtctgttcagatagaacacctttagcactcaagaataggttccagtaacttaaacacctgatgagtgaaaagggaaatgaaacctgagcctcaccattctcaagtcacttttttgaccagacccacatagatgcctccccagcagtctgtagtggcccttgctttccccctccagctatgggactcctaagtcattgccctcttactcacaacaatgatcttaggttaagttttcaggcaggacatgagaaaatcagcaggacagacaagtggcatcctataacactcccagatctgtgagaacagagtgagtgggagtgagaagagcaagaacgccaaagaaatattcttggcggcaggctggtggacgcagaaggaaggtttagttatggtccctcaacagctgcaataaacttgctctgtaagaagccatgattatgttgtattgtggatttgccagcaagattacatccacacaagttctttgtagaccatcagtaaactcagggcattctgagcaaataggaattagctacatgggcagagttttcaactgaaatccacgcctagttcccctaaagattgtttaccagcttttcccagagccagttacaaggagcataacatgacttgattcacaaagaagtaatggaattatagtaataaaggagtgaggaaaataaataacaggaatagacttggcacttttgaaacttaaaggtgttttgcctgagatgaacctgagaactgacctacaatgcttctcatactcgtaaacatggtgcaaagtttgttttaatcatacagaatatgtcaccattaattctttaagcatgcaaagagcatatcatagcaaatattagacaccccagaggtgtaaactatagcttagaaaaacaaaactcattggtggctattactttgcaattgttagatgtcaccattgtcatttcatgttatagatctgtgtcacaacaacatctgtacaaaaccaaacaccaccagctgtgctctttcaacaatttggagcaataattaaattgtctttagtaatacacctgcactgcagatagacaaagccagtcccataaaattttacatgcttatttaaattcttcagaaagtttcaatgaaggatccaaacaaaccaaaaatgctaaagtatgtcaaaattgccatgtgagaaaacaagtacactgatacaaattaattagccttcctcctttggttataatctaacaggctacatcatacttgctgccttagctcctgggctattattgcctatctgagatcacttttgatactcctgaggtaaaggaacaccaaacagtagtcatttatctgacaaaagaccttgtgtttttcttattctatcaatcattaaacaagtaatgtctttttatgttattgctttgacattcattaacactcacgtcagaggaaatctttgcaattaaaaattctattgaccatgtaaggttgttttgcctgggtttgttacaactgattttttttttttaacagaagcaacagcactgaggcaggtcaggaacctaccacacagttcagcttgaggtatctcttctgactcaaatgctgctggtaattattaaaaaaatattattttaaaaaaaatcattttttctatctcaatgtaaacgtcaatgataatcttgactgatcagcttagcttaagaggaaagaatacctcagaaagacaaaagtcagagagccacaagactcttgtgttgttattaaattcttcctcatggcaaatgtgcaaactttcaggagagtttcaagtaataatctgaaaagtgtgctaaaatctcaaatgtttgaaataagttttacataaggtccttgtgatttgataccataaacagaaacagagtaggagaagtgcctaacatgacaaaaggagaattttgaatataccatagagtaacttgttcacttccaaaccactccttttgtgggaacatcaccataaaaactgattatacatcatccacggcaatattatcatccagcatcgggagaaataaacctgaacaaacacatttctttttaataaagagcaatttagagggtgggagaagaaccaatttccttcttggaaaaatctctgtgttataaaaatgtcttttatttacggagtggattagctttatggtatctgtatttatatgccttctactt

SEQ ID NO: 5 - DNA, 2855 bp, Homo sapiens
aagccaaatcctgtttccaataccataaatccagtgaattggaagctttatgcacctctctgccattttccctcactgtctcaaatcactaaatgactttgtttattggtttggtttttagtttatcttctttcctttggctatattggcctaatgccaaataaacagttccttctcaggaagaaaaggatgagcttctaaacaatgagatcagacactgctggttatcagagagggtcatgagaactcctgtgaatagctgcaagaaatgtggttgctttcaaaaaatatgaattttaagtccaagtgactacaaagcaatgaagctgcttttgtgtttgctcaagggaagcaatttcatcacccctcatgcatgagagatgggttctggaggaattttccaatcttctcacttccaatgaatcaattgaatcaaattatcagtatagttttgggctcagaaaatacttttaatagttaatccagctgtgcatgtaatgacagccttgatattttaaaataaatgtcaaagtgaagctatttccacgcacttctatacatcatctgttcagctggtgaattaatataaaaaattaattttagtgttgacttactttgtattttgttcccatgagaataattcagccaaatcaatacacctaaaagttttttatgtatcctcagttcctgttctttcaaccttattgacctctggttggagccaattgacaagtaataaaatgtataaaaatataaatttaatttaaaggatctaatgtgaatggtgatgaaacacaccattcttggaccctaggccacatttataagctgctggaccattcagaagttgaagtgctttccttatattttaaaaataagtattactgagctacacagaagataaaatggcagtctagctaaagcaacattaaatagaatatttcctatcacctcaatcaataacctgaaaattccgacaactttgaaaacattgttaccatagtattaagaacaaaataattaagaaagcctttcattagtttgctgattataaatataactttggtgtacttttttcctgttcctatatctttttaacttcctctgttttgtctcttaaaatgccatcttagagttagtttttataattagatatctctctttccactcaatttctctcctgaagcttgtgctgagtccctcctacgtgcaaggcactgtgctagggttcctggctcatagctgatactcaaaaaacatttgtaacgataagatctttgaaagggtagcttaagatttctttattctttcaccagtaaatttacactactgtgaaaggcaagacatttctaatcagaataaatgaggttagatacaataggtactgataggactcaataaatattaatagaattagtgaaggctcttttgaaaattaaaccccctgaaaaccgcatttttataggtttatttattgtggtagttacagtacactctgagaaggagctacttaattcataatataatcatattcagtgaagttaattaattccagattttattatgttacttgagctcactgagtgctttaatctcatgatataaggaggtggaaaaggtaggtgtaacaagtcatcacagaagccaaggagaatgataatgtgcatctcaattacgagacaaggactaataaatgattgcctaattgtgggggctggaaaaaaaatcccaacacaaggaagcccaaagcactttagaaatcagaccagatatctaagttccaaagtagcagaaaaaaagaacaaacatggagggcggcaggcagagagtgccttgccagtaaccagggcttccaagataatggtaaacttatgcaagaaataaacctgtcttacaatggacacgttgactgtcagcaccaaggcccagcccactggaaatcagagttgctgaaatatatgattagtgttctgtttcatgctcactttcatgccacacctttcagtggaggtcatctttcaagtcattaaataatctaagagtgatatgtacacagaagagtttgttccttatcttttgtatgtcacacctggtgctttccatggactctttgttctagctttaacaaagttgtgtaaaggtttttttttttccccttcccaatgacttacttcaactcagttttttaaccctgtaacaatggcagttattgggaatgtctctccacctagacacacattagttgttaacaaagataatatagaaaaacatattttgatttcaatattctctagtttggttaaagattttccttttataagtatatctaggtacattgttaccgctctgttcctttcaagtaaacatattaattcaatattactttgtaatataattctgatttaaatatcttttatgacactcatgcacaggtgtttaaagtaaagcattatcgaggtaataaaaaattcatgattatcttcctttgtataattatagctgaatattatgcaataagttcaatagatttttattaattaatatatactaagaaaatattaattcaactgatattctcaacattgctcatctcaagcctgtgtgatagataagacatattcatcccattcagcagatgaaaaatgaaaacttccaagaaattagatgacatataaaatagaaagaagtggcagaatccacagctgggctcagtggctcactcctgtaatcccagcactttgggaggccaaggtttacggatcacctgaggtcaggagtttgaggccagcctggccaatgtggggaaaccctgtctctacaaaaaatacaaaaattagctggacgtggtggtgggc

SEQ ID NO: 6 - DNA, 2855 bp, Homo sapiens; identical to SEQ ID NO: 5

SEQ ID NO: 7 - DNA, 1803 bp, Homo sapiens
acccatgctactcaataaagaacactaaatgttaaaaagtgttgtttcataaaattatagtactagtaacccacatataacctaaagagcttaaaaattgccccaaatctcataattttctcatctaactaatccgatttacattcctgaacaccagaattattcattcattatattctttacaatgtttatattttaaaaatttagaaaaacacattaatgaaaagatttttttatataaattatcttttgtttcttaatatagatgtacccactttattctgacaatccctttgctgaagaacttctgaaggactgtggctaaagatcaaatcttaacatatttttgtcaatgaatacctggataatttaattattaaggaaaaatcaaagttttgtctacataaaaaagtattgcatatctatatgatagatattttagtcaataattgcaattttgtatctaaggtgataaatctacacagtaatgtatttatgtatctttctagtaggtattaatataagagaactattcattatgctaacagtgaagtctaaccctcaaaagaagcattatctattccacattaaagtaaacccagttaatacctaaaatgaatgttttaaagtacatagaaaaatgattcagcaaaaattataattacaaagctgataacggtcatttttcttttgttaataatattcaaatgtatcccttattaaaatatgtaatcttatttttaccacactctttctcaagtgtttgaatctttgaccatccccagaacctgacgccttgtcagcctattgcaggtaaaagcttttgaagatctaccactactcttgtcctaataaggtcactgacctttcaagctcaactcctaccacccccatcttcatttcctctcctatcattagtctgaaatggcatcaactacctctcaattttccttatcccttcaatgccccacctctttagtccaaattcacctaattaggaacttaatttccttttttctctgattgaggaattgataatagatggaccctattcatctttgattcacggaaaactgcaacaatgacctcttatttaggaaggataagcatttattctagaaattgttttgtggaggccaactacagaactaagtaaaagtgatgtttttatttttattttttagctaaaacagggaaaggagaatcattcaaatctgatacgttttgtttcttttacatttcattttacatagccgtagttactaatatttaaaacaagtagattttcctgtaaggcagaaaataattgagttctaatagaagatcactgagttagatttaaagaaatatattaatggaagttaataatttcttaaaaccggtcatttttaacaattttataaaaacaatctctcacaccagaaaagacatatgtgggctaggtatctacaagtgggagtcacatattaaaggagctatctcagtaattagctaactttactatgaaattatcttcttgactctgatgtggtagtttaactgtggagttgtctaacgtaaaaactcaaagcacataaatattccttgcaatattttttttctgaaacttgttggggggaaaagtatggccttaagagttagtggtagcagaaaaaccaaaccaacaattttattccaccactaaaatttttaatgtctaatttcatatcctattcattcagggagtgaaataatcataaacatcatagtaaagcttagaagtgacaatcataataaacagtaaagacaaaccaacttcatttt

SEQ ID NO: 8 - DNA, 3607 bp, Homo sapiens
cttacaggaaaagtctgataatatattaaacttgtttattacagcttcatataattttaaataatactactttttcctttgacaagtgcaataaactctactggtatagacataggttgattatcacgagtcaactgtgtgcttaaagaaacatgcccaaacaatcagcttttgacggctgcatctcccgttatttacactcaccatcatggtaataactcaccatttctatattaataaggcagtgaaatttattatgatggcattgaaaagttattaagtactgcatcccaagtgacaaagtaccacagcactgtcactatccaaaagcagttgcccattcatcacctgcctttccagatggtcctgcatctttatcgctcccaggttggtgacatcacctgttcacccactcaaaattgatgatgaaagcaaacagatctatcttacttacatttaccaaatcaatttaatttttacaggccatggcttggtgacatattgactgttttccttaatggcatctaccgtatttaatgcaaggatgtcatctttgattcctggcttgcataatgtgtcatttcataataaatgttttctaacatatttacttaatggaaagaattatgattgcttgtcagttctcttattaaacataatgataattctttttcttcttgagtatcatccattgggcaagaaatctatttttatctctccttgtctcttacaggagataatgacttggatataaagtaagctctaggcaagtgttagacataccgctgacccttctcaatcctggggctcagtgtaagggttttatcttatttatttgttactatagaagaactatagtcttaagggatgctctaattaacacatcagcaatagctaaagacggatatgaaatagcattgcaaaaggataacagacgtaaaactgattttaacagttcatatgatgtttaatctttacttccctgggctagtatagtcgcttctcttcagctagtgactctatatcgagtgcatgttaaaatacatatctgtgttgtaaaagttaagatgcaggtacagagactagtctactgactatccgatactcacatgtgacacctcctttccccaagtatgatttatagatcccatctgctgctctttggaacaaatgcttctctgacctgtgtcaccatggcattgtggtacatttctattacctaagggaattagcaattagatttacttttggagatgaaggcaaaatggcatcaagcttctgtatttctatttaatatccatactatatgggtatgttctggggcaatattcctgactcattcctcaccagtgcttatattcagagaggtgtgggagtccagagggaaaatcagtatgtggtctccctgccctctaagcccccacctgtgcaagtaaatgtgtatgcaagtaaaaacaacatcaaataaactccaagtgacactctaaagagaaaaattgggaaagggtctgtgacaaaggaaaactgagtaatctaaggtgtgtgtttctgtttctttaggtatttatctatcaagcatctatttctttagatatcatgacctagccaagagaagccttagtaaggtaatggagagagaggactggaaaaggttaaccggcttcagctctacactgctgacttagactgacagcaattgcgttccttctcttgctctctggttctctcaaacagtgttactgtagctaatctctccccacccttcattaaggctagatgaggtctggaatgaccttcagatttatcttgccctatactgaactggttgggtcccagggttggtgacttcctggggagattccaggaagatggaagaactccacacaggatcactctccatctgtgctcctactgtttagtctcaatcacgtgagcagaaatttatatgtcaacaacagcactctaggatggcttatatccctttggaaaccaggtccaggatttcctacatccctataattgcaataatactacctcaaactaggaaaaaaaaaacttgagttcaaagccaggtaacctgctcctacccgtacagtacattcacataaggagactcatactcatctgaaaaaatgcttcatgttcatctgtgattagtttatcctaattcattagtcagagtctttcccttaggtgaggcttccaggaaaacagggattcaggccagtagtgttgcgagaccaacaagaaatacctttttttgagggcagctggcctctgtgccagggtggagattctatgctggcatcacacacctgctgaattcttaccacaagaatctaaacaacgaaaaaagaaaaaacaaatcatgcacacctgtggctgccaaccagtgaggtttcattgccatgccatgcacacctgggaagcctgtgaggtcagctgcagtgtcagaggaaaacaacactctcagccagttggtaggaggcacaaagcatccatctcccatgggtctgtgacatgaactgactggccaatagccatagaaagtacatacacacacatcttctatgaaaggaaaaccctaagtgtaggcatggcaagaataaaagagatttcagagcagaggctaactgtaatcattatctgctgcgcactgtgcccccaggaagaaggagtcttcttgcatcaactcttaggtcctcctttattagccaaatccagtcgctgggtaacccagagaatagaaaaagactatattgaataagtgtctctgtatgactgaaaaaacagaaatgcagagtgctggattatgaggagagtgagaattttgagaggagagagtctgaaagcagcacctgctggatgtgtgagatactgaaagactaagaaagtgaattgcagaaatatacagggatagtggatttaagttttgcataacattagtcatggaattcccctgtgatgtttctagaaaagatgtgatttggtttttaattttttttttgtgggggtaggcagaagttgatggaccttaaacaattttttttgtagttactgccaaggtgtaactataaggcacccagatttatatgcttacctaaagaaagcatcaggtatcaaatacagtactggtcgattccaaaatgataaatatttaaaagcaagaaggcatgtgtatttgcctgtgtgtgtcgtaggggatggtatacaagattgtgcagtgtgggttctagaaacattgtttctgtatcaggtgacattctatggctgacttcctaggattagattaagaagtatcaacaaattattttctgtatcttcagcagcactctactggtggaataaaagtcgggtctgaaagctggtggaataaatgctcaacagtgtaaaggggctggatctgcaagtagtctattggcagtgggtggcatggccaacaataatacaattaactttcaattgttcttgaagctgattgtctggttcctaggcatgtatattcctctgagaagatacctgtgagatctccttaggtcatgtggacccagctcacactatgctgtggagcacttcagtgagataagctatttctatgaaatgtttg

SEQ ID NO: 9 - DNA, 2212 bp, Homo sapiens
ggagaaaatatttgcaaaccatatatctggtaaggggccaatatccaaaaatatataaggaactcatgcaactcaatagcaaaaaacattaataataaaataactcaatttaaatatgggaaaaaggacctgaatagatgttttttcaaagaagacatacaatagcgatcaagtatatgaaatgatactgaatatcactagtcatcatggaaacgcaaatcaaaaaaccacaatgagatatcacctctcagctgttagaatgactgttattggcgaagatgtggagaaaaggggctctttatatactgttggtaggaatttaaactggaatagccattagggaatacagtataaacagtcctcaaaaaattgaaaatacaactcctttatgatccagcaatcacacttctgtgtatatttccaaagtaaataaaatcgccatctcaatgagatgtctgcactcacatagtcattgactgcaacattatcaacagtagccaaaatatagaaacaacctgaatgcgaaggatgaatcattttaaaatgtggtacatacacaatggaatactatttagctttttaaaaagaagaaaaccctgccatttgggacaatgtggacaaacctggaggatattatgctaagtgaaataagccaaacacaaaaagacaaatactgcatgatttcacttatatgtggaatctaaagtaaaaaatgataggaacagagagtagaatggttgttcccaagaactaggaggtaggggaaatggggagatgttggttaaagggtacaaactggcacttgtaagatgaataaattctggagacctaatatacagcattgtgtctatagttaataataatgtattgtatacatgaaatttgctaagggagtagatcttaagtattttcactacacacacacacacacacactcacacacacataactacatgaggtgatgggtatcttaattagcttgattgtggtaatcattttacaatgtatatgtatatcaaaacatcacgttgtacaccttgaatgtatacaattattatttgccaattatacctcaataaagccaaaaaataaaaaataaaacaaacacaaaaaatctgttgcatatgtctaaaatctcaaattatttcattcttcttcattatcaagcacacattatttgtcagcctcctgatgctgaccaccgttgttaggtatcttagaagtgcaatgttgctgttgttgccattttaatgaatattcctgtcttagttaacatagattgcctgtttcaatatgtaaggtgtcactgtagaatctaaaatcaaacttaaaagtgtgagaactatttatttttaaaatgacacaaggaagcacatcttcattgtaatatcacattttaggattacattgttggaacataatgtattgcccaagcattgattcaaaattagttaatgaatttctgactccagtgtaatctgtttccactattcctttccacctatttaacacagcataagagatattcactctctactcatgtcattatgttctatgtgtaaatgtatgtatgttcgaaagaagaaaactaagttcacagccaatattttaactgattaaactaatttggagcaattctggctgagatgaacacaaagccgggtctggcacatgacaggtgcatcaacaactgacttatacttgcagagttttgtaatggccaatgataataaagatcagcaaagaaaatgtgccacaaggcaatattttaaagaagcaaactttttacttttttcaatggcttatctcagagtattacaaactaagccacctaaatttttctttcacgtaaacaagaagtttgtgatctctcctgaaccctcgttttcattgttagttagaaaacggtcagtgcttccatttcaaaccaacccagagtcaaataattagaccatccagagataactgaacttctaggaaacaagaaattggaaaaagagcaataacttataaaaatcaatcgataaaatataataattggcttttgccctaataaccctatgctagatctgcattttagccaagctgcttctgctgttgtcttgttaacatttattgggttcttctgccagctctgtgctaagctcctgacacacaatgtcacattttacccttacaacaactctaattggttagatacaattattattcc

SEQ ID NO: 10 - DNA, 2462 bp, Homo sapiens
ttagacagatcaatgagacagaaaattaacaaggatatccaggacttgaaatcagctctggaccaagtggacctaatagacatctacagaactctccaccccagatcaaagtaatagacattcttctcagcaacacatagcacttattctaaaaccgaccatataattggaagtaaaacactcctcagcaaatgcaaaagaacagaaatcataacaaacagtctctccaaccacagtgcaatcaaattagaactcaggattaagaaactcactcaaaatcacaaaactaaatggaaactgaacaacatgctcctgaatgactactgggtaaataatgaaattaaggcagaaataaataagttctctgaaaccaatgaaaacaaagacacaatgtaccagaatgcctaggatacagctaaagcagaatttaaagggaaatgtatagcactaaatgcccacaggagaaaatgggaaagatctaaaattgacaccctaatgtcacaattaaaagaactagagaagcaagagcaaacaaattcaacagctagcagaagacaagaaataactaggatcagggcagaactgaaggaaatagagacacaaaaaaactcttgaaaaaaatcaatgaatccaggagctgtttttttttaaaaaaggatgaacaaaatcaatagacttctagctagactaataaggaagaaaagagagacaaatcaaataggcacaataaaaaaagataaagaggatagcaccactgaccccatagaaatacaaactacaatcagagaacactgtaaacacctctatgtaagtaagctagaaaatctagaagccctcagaaataacgccgcatatctacaactatctgatctttgacaaaactgacaaaaacaagcaatggggaaaggattccctatttaataaatggtgctgggaaaactggctggccatatgtagaaagctgaaactggatgccttccttacaccttatacaaaaattaattcaagaaggattaaagacttaaacgttagacctaaaaccataaaaaccctagaagaaaacctaggcattaccattcaggacataggcgtgggcaaggacttcatgtctaaaacaccaaaagcaatggcaacaaaagccaaaattgacaaatgggatctaactaaactaaagagcttctgcacagcaaaagaaactaccatcagagtgaacaggcaacctacaaaatgggagaaaattttcacaacctactcatctgacaaagggctaatatccagaatctacaatgaactcaaacaaatttacaagaaaaaaacaaacaaccccatcaaaaagtgggcgaaggacatgaacagacacttctcaaaagaagacatttatgcagccaaaaaacacatgaaaaaatgctcaccatcactggccatcagagaaatgcaaatcaaaaccacaatgagataccatttcacaccacttagaatggcaatcattaaaaaatcaggaaacaacaggtgctggagtggatgtggagaaataggaacacttttacactgttggtgggactgtaaactagttcaaccattgtggaagtcagtgtggtgattcctcagggatctagaactagaaatactatttgacccagccatcccattactgggtatatacccaaaggactataaatcatgctgctataaagacacatgcacacgtatgtttattgcggcactattcacaatagcaaagacttggaaccaacccaaatgtccaacaatgatagactggattaagaaaatgtggcacatatacaccatggaatactatgcagccataaaaatgatgagttcatgtcctttgtagggacacggatgaaattggaaatcatcattctcagcaaactatcgcaagaacaaaaaaccaaacaccacgtattctcacttataggtgggaatcgaacaatgagatcacatggacacaggaaggggaacatcacactctggggactgttgtggggttgggggagcggggagggatagcattaggagatatacctaatgctaaatgacgagttaatgtgtgcagcacaccagcatggcacatgtatacatatgtaactaacctgcacattgtgcacatgtaccctaaaacttaaagtataataataaaaaaaaactaaacaaaaaaaaaaaaagaaaatctagaagaaatggataagttcctggacaaatacaccctctaaagactaaaccaggaagaagtcaaatccctgaatgcagcaataacaagttctgaaattgaggtactaattaataggctaccaaccaaaaaaagcccaggaccagacagattcacagctgaattctaccagaggtacaaaaaggagctggtacgaatccttctaaaactattccaaacaatagaaaaagagggactcctccctaactcattttacgaggccagcatcatc

SEQ ID NO: 11 - DNA, 2604 bp, Homo sapiens
agcgaaactcgaaagaaggaaagaaaagaaagacagacagacagaatgaaagaaagaaaagaaagacaaacagacagacagaatgaaagaaagaaaagaaagaaagaggaaggaaggaaggaaggaaaaaaggaaggaagggagggaaggagggagggagggaaggagaaaagaaagaaagagaaagagaaagaaagaaagaaagaaagaaagaaagaaagaaagaaagaaagaaagagaaagaaagaaaagaaagaaagaaaatttctttcaataacattgagagcagtagtttttaacctttgtgaaaaatcatctagagtgcttgttaaaaatgcaaatacacagggcttgtccccagagatttactgtgactgtagggtgagacctttcaaatgtttgaacgcaggagattcaaagatcacatttggagaagcacctatctagagaaaaatcctacttccaagtttaacgttggagagctgttatctgaattcatctgggaaaaaaaagggtttattcttaataatggggaggtggcagaaattgcctctttaattttacccattctccaagtgtcaaaacgtaattgataattaagattatgttccctttaatatttttcaaatttagtttatttagataatgaataccacatgtccttcaataattttaaaattcagttaatttaaaaattgtataaatagattctgagatactaaacaaatatacagtcaaattttttatgttttcatttgaacattctggggaaaataagcatactttagaagcaagcacatataaatgcataattcagttaggttttttgttttaatttgtccttggcattgatgaaaaacaaattagctactctccttgggtgcctaccagaaataatagtaacaagttgagcttttttcctcctcagtaatatgttcctttgtagataagcacagtggagaggtaaaatgatgtataaaaagtttactgttttttttttgttttgttctaagatgtaaatgataggggaaggactgggagctatttcagaaatatttattccattgagacaatgaattttggtcattagaagaaagaatcatggactatcaaaatgaaacaaaaagacagcagatttatttctatagcaagtgtctgtacataaagagcaggcacacctcacaacattgtacatttattgtcttacaaaatgaactggggttgaggtagatgtagtaaggaaaaagctttaatgatgtgaggcaaatttttaagagaggagatgtttttatttttaaagaattcaataaatataatgatagtgttaaatgtttttgttgttttttctctcaaaactatttgaaccacttattaatctccagtctgtttcatcaatcttttatgatgtgttacaaggagtttctcaaatttaaacttcattacatcattttatagttataaaccctgaccactataattcttggtcctaactaggctatgaaaagaggaatgtattcattaggctagtacagtatgggaagcctgtagtttaaccaaggttaaaacaaataagatgccaaagttcacaattttaaataaactcaagaacctgaaaaagataatttaaaaacaacatagtgcatttctatagtttgtcattatttcatctcttatggaaatatgcagcaatgatggtctttttctatatctagaaaaaatacataagtgatttttaacagtaagaagttatttgtgtttggatttccttcgccacagcttccttaactagctcctgggtagaattcattaatttttattaacagggaaaacatttgttcccgtgacaatatttgcattgcctgaatgattttcacctctgccacaaggtgtactatcggctcatacatcacccgattacttggagagcagaaagaccctcagtcattagtgagtgaattaaagatgatcttaagagctattaatcacagtgacaaaagccttaatgtttctgtgaccctagtggagcctggggacccaaagggaagcagagggagagaaaatggaaacatatgttttctccaaaataacctaccttaggagaactgtcaaaaagagagtcacatcaaactaagaaattctgccagcaactggcctctggattatgtgtctatttatcttcttgtgaactcatatttccttttaaaatatatattttcttgctgtagacaattagagggccaaaactagtgtcttctgtatctttgatttcttaattccgtttatttagaacgacttgtgcttctgctaaactgcatttcttgaaaaaattcaacataaattgcattcagtctaataaccacataaatcagtataaaaacaagcagacaatagcagtaaagtgccattataaacattaattcaagcagatacaatattccaataatggaccaaaacggtaacattattaagcactacagtcggttacaaaacttgatcattcttcaatgtatctggctgtatttccctggatgtttacaagattataatggcatcaactccactgttaataacataggcacttgctggaatttcaattatctttca

SEQ ID NO: 12 - DNA, 1437 bp, Homo sapiens
aagttagccagagccagtgtctgttgactgcaaccagagacccctaaaaacacccttttacacatcaaatgcataaatgttaagtaattacccagagttgctcaatgaacaagaaagtggtaatctcatgacttctaattcagtgttctttacactctgccacagataaagatgatcatacaacactttttaaatatagagcttggttttccaatggtaaaaatggctttaaagttcaacatcattataggagtaatttgtcctcaggaagaaatgaagaataatttaattttggatagttataagtatgtcatagaacttttagaaaaatgccactcatttccatcagcaatattttcagattgttataaagatataaacatattaaaccatatgcttcatatattaggttatcaatgcctactgttgactaactttcccaaatacttctttcctgaattcagatgccatataaaagaaagcaaaaagccactgtaaattaaatgaattgagaacactaacaacaataatggcaaccattatttaagtacttaccacacaccaggcaattcattggcatgtatcatacgatagtaaattctcacaacagctttgcagagtcagtgtcaatatcccattttagagagagtgaactgggacttacagatgttaagtaatttgatcaagttcacataaccaaaaaacgattaaaactattatctaaagcccttatttttttctgtgcataccatatataatctcagaaacactaaagagagatgtcagtatagagctttatccctccaattcccagaggcttagacaaagaaaacgttggtgaaaccattggactctatgacaactagaactgcataaatcataatcaatagaaaaactaaatttatctctagaatttggaaaatcacaggacatatatgttaaataaagattctgaatattacagtgattgaaaagacatttacccccacttaatgcaataatttgaaccagaaaagccttgcattttgaaaaagtttagaaatctttcttttaggtttacaaagaaaaagcggccttcattttatcccttagacttctgtgtcctccacctgctacattacacttcaagaaatttaacttatatcaatcaaaatttgcttcttaatggatgtacatctcaaactcaatagcccaccctttacaaacactgtacaacccaatagtgaaagcaaaaaaagctatgtagttatttctttaaaaccttcagcagttaagaagaaggagacatatcaaagcatattttttccatctggctccagaactttagttcatgggcaaagtatatggtatttgtattggcaaaataagttgtgatttcaaagcatcatctagtagcactgtaacatagaccatggtgctaaatgaggcatgctagtg

SEQ ID NO: 13 - DNA, 1770 bp, Homo sapiens
tgcttagctggccctggatttcaggactgggggtgggagtaccagggagagctatgcgatgtgttagagacactgaaagttttcattttgactccttaatgagagcgacctttgacacaggctgatgagttccatgcctgtcctggtgtgctttttgaaaacatttgacactggtcacaggtctgacagggatatgactaatgaagtcgtggtataaaaataatgggctgcaattgcaagtcatttgcatgaaaccaaactccttaaaaacttgtctctgttcactttctgttttctaaatgagtcaataatatgaatcagatgggttgaagggaatagactcccaaatattccttctataagtttggtaaattagttggcatttggtgcaatttttgttgccttgaggcctggtttaaattggactgtagttactaaattgctagggcgattgcagaatgtgtaggataatgcataataccttgtttttcttaagcgtttcgtcagagagcttcttgtgaggtcaaggggcccaggcagcgctttgcagaaaatatcaagcgagagtagatgaagaagaaatataaggaggttgtgggggagggagaaaggaaaggactaaataaaagaaaggaaaaaaagagaataaatgtagaaagaagaaataaaacaaataaaaaagagggcaacatggattggggtccaaaaacacaaaaggacaggaacaggaggaaaaagagggagatggggaaaaacgggaaaatgaacgcagagtggttaaagaaaatggggaatagaagaaaatttttaaagggagaaccctaaaagaagaagctaccatgttgacttcagtttatcctgtcaaactgtgtattatttatcctcacacaggttaagtatccactaaacaaaactctgggctaatagcagctcacagctcacacactagggccaatccttcccttcctttgtctctgcacaaccctgccaaagttcctgatgtttcatcctttcacctgtagctaaaaacaccttcagagaaggttttagcaaaacttctgaaattacatttgaccagctgcgtcaggaagcagcagccaggaggaggcacattgtacaagttccttcctagagccttcctcctgtgggactgactattcatgatttaaacacttagggttttctttatgatttgtcagtttcagcacatctatttggaactttgttttgcttactggtatatacgacaaaacgaaatatcatgtgttcccaaacttaaataaaaagaaatacaaatgatttacagatatacacacatagaacaaatgaataaaggaaaggttacatggcacagaggaaattgtacaagaggtttggaaatgaaccagctccagttgctctgtgaccttgggaaattgtcttaatctcactgagcctcagtttcttcaatcacaaaatgaaggattaaaagattgttttacaaagtactagcaaagtacctggcacaccatgagtagctaaaatgaatgaatgagcaggcagagtagtaatactctcttttgtgcctgcctttcaaggaagtatttctcacctaccattcatacataaagaaatcaataataattctacttatatccctcgataacactgaaaatccaggataaaacatgcatagactccataaactaaccgcattgcatctgttggggttgtacattctactcccaaaaacctgaaatccaaacaactaacaaagtgaaacatgttgtc

SEQ ID NO: 14 - DNA, 2256 bp, Homo sapiens
gagtctgaatataagctctatgagagcagggaccttgtcagtcttattcacaatatccccagcctctagaacaaggctggcacatagtagatgcacaaaaggtgtttgctgaatgaatggatgactgagtctgtgtggggtaatgatagggctaaggatgggactctaaactcaggtttcctctgtgggtttcacagtttactggtcttaagaggagagtttcctaaacttgccttatgataaaaaccaccttcagcatttgttaaaaattacccattcctgtagattctgagtcagtgagctgaagtggagctgataaatctgtttttgttgatactgctgctgctgcggtttttaacacatgcttcaggtggttctaagcttaggaaaccttgcccaaaggataccattctgtctcttgggaaactgcctctatgataggcatttgcaaattcaccaagagtgacagatagtgcctcgggcactgccactgtctcttccaatgcaatttgtacattatgccgaatcataatacaggccttgtacttctgcctcagctgacttgtctgtgggatcctgcagctctcaaccatgcggaggaacgcttccattcacacattttgtccccctaaccctgtcttttctgagggtgcatcaatgtctgatgaatgaatgaatgatggaataaacaccaacatgggcaaacaagcaagtcaggcagttgtgaggagtggaagtgtttcataagcttcagtttttagttgcacgtagtagcatacaaacaatggcaaaaagaatattaacatgtcctgtttccattgctttccttctgacttaaagagcgttcctataacagctaataatctctggacctcaaaatcactatatatcttttttaaaatattttattttccttagttataatggaacagatatggtgaccttagtacccaaaggattattttagtcttacagatatctctctcttccttattctcatttttattcttctttcttcccccagtctgataattatgtccagaaatcagtaaagaacatcaatcttggttgggataaatttaagtagctggaaatcataatatctatgatcctccagagtctagaacacggtttctaaatctcaacgctattgacattttgagccagataattctttgtgatggggagctgtcccgtgcattataaaatattgagcagtgtgtccctggcctctacccagtaggtgctagtagccaacctcccgaatctctagttatgagttatgacaaccaaaaatttctccagatattactaaatgtcccctgggaatcaaaataactcccagttgagaatcactaatctagaatgattttttaaaacaatgtgctttttaacatattagtcgatatttggtatttatgttaaaggatatattaatcaaagttgcttgaacatcaaacgaaaatatggacataatttcttaaagaaattgttggttcaaaagaatcatggctacccctttagatgtgaagttcaagagatatcaggcagaagctgaaatggaatcttgacattgctgagattttacaaaaaaatttgaaaggagggtgagcttgaagactaactaaaatgtctcacttttggcaaaaacatccaaagtgtaatgcactttgccaacagctttgcccttggctttttgtcttgttttttctttttaacttaagggtagttcaagggtagttttaatctgtatgttcctggttcctataatctaaaaatcatccaaatgctgttttataagagtatctcgtaagctttgatgtccaagaagcctgggggccctttttttatgtgcttattttcttttgaaccaggaaattgtgttttgccaagagcaaatggaacttgagcctcaaaagatttgagctctgtttgaaacatagaacccacaaattctctacaggttctcaacttgataaaatacgtgctttcaccttccctcagaccaaacctctgctggttctactttaaatcaccattagcactgtcctatacacctaaagatttccccacaagagaaagggttaggccgtaagtctgaattcctgacacaacactattttcagctggactcatcatcaaaatgctctgcaaggtaagagaagagaagttaaatgagaagacagagaaaaggtgagagattcaactaagtaactaagaaagtcctttgactttccccaggaccaaatccagcaa

SEQ ID NO: 15 - DNA, 1894 bp, Homo sapiens
gcttggttctgattaaaaagtgttttaagtgattcagccaaggttgtctccatgggctcaggaagtaaccacagccaaaataccctagtgagggcagaatatgagaggccgataatacctattgttaccttcgtcaaggtgcagccaggcaccaggctcatgggagcaagcaaaggtccaggcctctgacaccgcagattctaaagcagccagagctagcgataggtactcaacaaggaactgagatctagtttttttaaagtatctttattttcttttctgtaaaataggagatagaagactaaacactaggattttatgctattttgaaaatgaaaacgtagcatgagaaatattgcctgatataggaaaaagagtgcagggcttaaagtctagattcaaagagctctgcggtcttaagaaagtcactttacctctctgagcttcacatttctaatcaataaagtgatgctgtgaggccagctgctctccaatttcccttgcagcttagaaagcctaagatgtctactctagctaaaaaataaaccaaccttataaagaatttaaaacagataaaaattgtctggtatatcagctagtgttggtgaattttcctgttaatttgaaactttgctctatttcacatttcttaacctcttaattgagaatttacataacttgtcagcgtctcactatcctaatctatagaatgggcatattaatagcacctgcctcattgagttgttgtgaggaccaagtgcactgctacatgtcttcagaacaatgcttggcatgcagaaagtactccataaatgtaagcaactagtagtcataatagtagtagcagttgttgtagaatagctctttgatttgattgtttataaatgttttcaaaaaataaacaagctggccgggcacggtggctcacacctgtaatcccaacactttgggaggccaaggcgggcagattacctgaggtcaggagttcaagaccagcctggccaacatggtgaagccccatctccactaaaaacacaaaaatcagctgggcatggtggtgcatgcctgtaatcccaactactccggaggctgaagcaggagaattgcttgaacccagggggtggagtttgcagtgagctgagttcgtgccactgcactccagcctgggcaatagagtgagactctgtctcaaaaatgaacaaacaaacaaacaaacaagctaaggtaggagttgtttttgtaacctcctcaaactgtcaataaataggtgcccagaacatctgtagtgctagctctgcactgtcactctcagccctcaccattacctgcatcattgacaggagaggtaaggcaaaaccctataaaaagaagaaaaacttccttacgactccaatcaagagacaagacatgagataaaacattggccatcaagtttaaaagatgataatctgtattttaagcactccaaactctctttcttcatactaaatattttgaactgtcacacagatgccaacagaggtttgtttgtttgttttttgagaagaagtttcactcttgttgcccatgctggagtgcagtggtgcgatctcggctcattgcaacctctgcttcctaggttcaagcgattctcctgcctcagcctcccgagtagctgggattacaggcacccgccaccatgcccggctaatttttttgtatttttagtagagacagggtttcaccatgttggccaagctggtcttgaactcctgaactcaggtgatctgcccccctaggcctcccaaagtgctgagattacagggtatgagccactgtgcccagccgccaacagaatcagttcatttttaaacgaaaggagttagactaacatcacttccagccattggactcc

SEQ ID NO: 16 - DNA, 3770 bp, Homo sapiens
aaaaatcatctaggaacaaaaaaacaaaaacaccaatttttagacagataaaatgctgtctcctaaaacacacacacacacatacacacacacacacacacacagagacacactggtcttcttctgatctttcctttagtgtaacaaaaaagttattccttaacatatttcccacctcctttcttcatgccttctcattgctttcccaagctgctcattccaacaatgcctttaactctgatgactcttattaaacacgactacttaattaccatcaatctattatgctatgtacttcaccctccactgacacatctgatcccattatgtgcctgacctttaacggtcatcaaatcaaagtcattctggtcagttcagtctgggcctgacacatgcccaggtcacagagatgaaggcgcaggggcctagcataccacgttaccctgcccttcacttaacacctttgtcaatagaatttcacaactgggaaactgccaggtttctgcctggcttgaataaaatgcccctccttttacagaaagctgaaataaataattacacacatgctgaccagcactggcttttatgtaattggtgcctctctaccagcatgacacactgccttcttggatgtttgtgctgaaaaatctttgcttggaaagtgagaatttattgtgcagccttctagaatatacccagcactccacactggctgaaaatcccctaaagatataagagagatggaaagacaagttgaggatggccacagcaacaccaccaagtgggtggccgccttccactgaaaggccagtccacggaagcattctgggttcacagacttgtgtgctaccctctgatgggaacagctgttcgggctgctctgtctcaaagctgcatgctgaaaaacactcccactcaacctaatgagacagccatttagaaaagaggctcttcctggctagtagggcctggtcattgttattcacccacaggctttccctggtggtgaacatggaactgttttcccagaccaaacatggaccctccctcgaggtagccagacacactgtctggtgtgcacaaacattttgacttctgactggacagctagacctggcagtgtcttccccttgctttactttactaatgcgtgtgtatttttaccagcccctctgggaaaactgccctgctttatagcaacttgtcactgacaaggaatttctcatcaagattgaaatctctggccatccccaaagctcccaacccttctgagtttctgagctcaggacaaggagatttgtccttggaaaaaaaaaaaaaaagaaaggaagaaggaaaaagaaaagaaaactggctttaggaagcagtattgtggttttttcatggctgtgatgaacgaaaaccctcccatagtggcccaaaggcaattacaaacatttggcatgtgacaaattaatagtgagacttatttcttagaagaacaatgatttgcttagatttttttaaatttctagattctctagaaattttccaatggaatgtgtattaccttattctttttctcccacctcctccaaaatataccaatctgagatgatgaactacatcacctatttagctccctcttctcactctcattcctgacaggttaaagctgttttcacaacagttttctctttttgccaggctcttaagcattgcctattagtaatacataccaaatttacttaaaagtcttaacatagggcccataaagcaacacaaccctctaataattgtatcagccatcgggctgaaacaagttcagttccctgaaaaataggaagtttcactgtgtaagaaatagaaacataaataataaatcagaaaaaaagacttcaatcccattatcatctttcatctgaaagactgaaattcacagtctttccacaaatcacttcacttgctatcaaatgtcttccaaaagactctaaatgtcttctaaattgaagttaattgttaaggaaggatacactcaggtgatctatcattaggcccagtaaaaatatttggcaatgtgagcttcgtttccatctgagaatctcaagacaattgaccagcaatcagaaattattcccacattaggagagatgatcggaagtacagatcctacagtaaatacaaacctcccatttcagacccaactcagatttattaccaaaaaaattacccatccaaaaactagcatgttgaaaatacagcataagctattatttggtatgtaacttcttatattacaaccatttaccacttttatttccaaaaactcattttattcaaaagtttccttatgtaagtgatggttctgaaggcacaaaaatatctccaacattttcctgaaatttttattggtttaaaagtgagtaccagaaagaattcagataagcatgacataggactggtgtgtttataacagcagctgtatctgtttgcatttgtggtgtaacttatcctcaccctcctaaacctgaagacccatctctactttgattgacaattgtatattagaggaattctcacggtaatatgtacactatgtaaatttctctatgtctgactggcataaacagactttgcccaattaaagagttaataatcattgagcggagcttgggagcagaaggtaccctgttgctttaagatacatttgccctcaaagtagttaacaggaaatttaattcataaagtcttaggaaatgtcacatctgtaactattcctctctctctctctcactcaacaaaaagagcctcgactgatctcattagaacactgagatgaactagcctgtggtgagtagctcagaggtttggtttgcccagggcctgacctccacaccagccttcctctccctcgtgatagcaacaatttcaagttttaaaggattgagcagcctagcttttttccttacatttaaaacacacctggtttctaggaacagtcttcagtgtttcatcctttacttgattatctcagaaatgttggaggccttgaagatcctggatgtcttttctctgctccttgacaattccatgtgaattttaaggtttcttttctctttttttttttctctttcatcttatccctgcttctctttttcccaaactctcaaataagtggggttttccctttctcagttcttaatatgtctccataaacatcttaccccagtgagtacacacacatgcacaaacacacataacaatgctatatgagggttatgtgaatgttcaatgtgtcactaaaattgctgatgttggtctgtatttgtaataccttaaagaaggatcaatcaattaatcattttaagttctgcttaaaacaaattacagcagtcaaaaaagtgatgcacaaccccttaaacaatgctaatgtattcttgtgaaaggatccaattgacagttttacgagactggagtctggtgaaagcaggtgtacacggcccagcaaaaatggcactatgtcgtgaggtttgaagggagatgcgaaagtcattttatgtggttggcctctgcagagcctcagagaaatgaggcagcagcgaaaagccatctgctgccaagatacaagcacagctattatacttctcgctattcacatgatcatacctgaaactgatatggagtgacattacccagcagcttaattaatgcttttctacactgcatgaaatacacattggcatcagccaagcaaaggaaaaatacttaagtggattcttacattgaattac

SEQ ID NO: 17 - DNA, 1419 bp, Homo sapiens
ccgcggtcttgctaatcgcatcgccttttgagactttacgagacttctggggggccggtagctaagggagttactggatgcaactcgttctctgctgtgactgaattcccgcgcgttgtcactttggggaagtaaggtaagggtggcttcggtatttcaggctttagacatgctgcgcagacccaacctgcacttcctttccatccatcaggaccatagcaactggcatttctggcacggcccaggtgctgtcctagctgcccaagaaaacggctgggaagtctccccgcgctgccagagtcggtttggcttttggttttcttttcttaaagatggaaataaaattcaggggcgttaggcactacaaatgtcaatcttcctgcctttcattttatttttaattaaattttttaaccttcccaaatgatttacaacgaatttatcggtttcaactactgtataaaatacaagccttacaagttgcaaactgtgttgtgcattcaatctcggtgtagattcagtgggtattttcagggtccgtggtctctggtctaggtccatgggctcctgtactgtagtcaaggccaaatcaggacttaagaccaccaaagaaatttacaaaagacaaaaagaacaacaagctaccgagaaaacaactatttatatgtaatttgtttccattgtaatgttttctcttgccacaataaaggtagttttgttttttaaagtattgtagtcacaactccccttctaaacccagaaaaagaaaaactggtgcagagcagtgaagttttcgttggatgattgcaccgcaggggtcaccaaccatctccgccacgcgctactcgggggaaggaaatcgcgctttaaaatacaaaaatctgtagttttaatatgatttcctttattacccagattgtaactgaagaattgcaacgcagcgcttctgtctgggaaaggcagctgcggcagcaacagctggagtagtgcaagcgggaatctgaggctcagtaaaagtttgtaagagttgagatctagctatcagaaagcggcagcggcatccttagagaccctggggacaaaataaatgggtctcccaccagttgaatgacaagtatcatcagatcggaaacgaggctcagtttctggatgctgagtggactgtgcgtgcgggaatattttaagacggcatcgtatgtcttgcttggcaattaaaatagttgaaaaagttgctcaagaaaattcgaagggttttgtgttctgctttcacttcagaagttatcgggcagtgagttgcggcgcgaagccttaagctttcacttcacaacttaggcatggttgaaaagggctacccacccccacccaccatcccactcccgccaactcccccccccacacacacactctctctctctccca

SEQ ID NO: 18 - DNA, 2220 bp, Homo sapiens
atgattttgatatttttgaaaatttttaagacttgttttgtgacctaaaatatgtctatccttgagaagatccatgtgctgaggaaaagaatgtgtattctgcagctgttggatgaaatgttctgtaaatatctaggtacatttggtctatagtgcagattaagtccaatgctgctttgttggttttctgtcttgatctctccaatgctgaaagtatccaatgctgaaatcaacagccattattgtactggagtctatttctttctttatctctagtaatatttgctttacataactaggtgatccagtgttgggtgcatgtatatttatgatcgttatatcctcttgctgaatgaacccctttatcattatataatgaccttttttgtctttttttatagcttttgtctcgaaatctattttgtctgatatatgtatagctactcctgctcttttttgttttccatttgcatagaatatcttttcccattcctttattttcagcttatgtgtgtctttataggtgaaatgtatttcttgtgggccacagatcgatcattgggtcttgttttttaaaatccatttagcacctcaagagcaattcctcagagcccagcacagcatcaagatttgcccaggaattgcagtccttgtgacctagactgcctttcaagtttatttagaacccccagagcactttatcccacagtggtggggctagccagaactcacgttccacccactggcatggacaattcccctctggctagggctggtctaaatgctccctccatgagcaccacctgaattctacccgatgttgctttccactgtgacaggcagcactgaagtccattccaaagccccacagtcactttcctctccctccccgaagcacacttattctctctctgtgccacatggctgctctgggggttggaggagggatggttctgactatccaggactgtctttcctaccttcttcagtgcctctttctttgctatgatgttaaaaccagggactgtgattgcaaacctgatctttcgttcttatgaagatgatttcttgtgtgaatagttgttcaatttggtgttcctgttggaggggatgattgctagagggttctattcagccatcttgctctgcctccaggaattttttttttaacaagatgtatttgtacagaattctctcagctatgactccctgttgaagaattttctttctggtatagggaaggcatgtttcacatggaaggttttatttcctgttttcaaggcgagaaagggaagattagaatacacttattgcatctgctatttttcaagtgtctttagcttaaagtaatcctcataccaaagtggcatattttggagtggcatattctgccactctttattaccattacatgaatatccagtctgaagtcaattaaggttacttttagtttgtttgtgattcaccagtttttagaaggagaagttctgctgagtgaagttttaagcaaaatcttgaaattcttttaactagaaatgggtttttatgctaacagagagagtcacacaccatattaacctcaggaacctagtcttacctatctagtttgtgcatagacagacttggaaacaaaagcatcaacactcactgtaaatttgagagggctgattagtttcttaaatgattttctcaggggttagagttacacaaagaaagctaattgttaaactatccaaatcttggctccttcaaatgaagatgtaacaattatttaatttaaaaatcacccgaccataatattggattgtaataataagtaaatttgatcacatttgtgtagcaatccctagagaaaaaatggccaataaagaccaaatcttcccctttctcaattactgtggcagaattactggcaaagctattgcaaatctgggttttacaatctccttttattaaatccacaaatgaacgtgaaatatgggtgtatatatatagctttcagagggccgtcaaattgatggggtatatgattaaggtgagataacatacacacacaatcagatattctaagcaggtcttattatggagaaagatgaatgctaagagaaataagttatttcatgtgtctggaagcccagacaccctcatattaattctaatccacataaatttctcccagttgactttgttgtaatgctgcacaataaaatcaagtctaaa

SEQ ID NO: 19 - DNA, 3060 bp, Homo sapiens
agggctcacttagcgacatgggggaggccaggtgtcctgattagtatagctaagagtcttgcaaaggcagttaagtcatttattaaaatggaaaggtaaatagcgactgctaagaaaattagcatttacccatacatttattacactaccaacaataatttactgtacacttactacatgccagaatttggagttgggatcctgcagtaaatttcatctaggtcacggagtcacttgagtaaaagaaaaagtattggcccagagtggtgaaattagcaggagactttcttggagctacataattgacctggaaaactatatatgattcctacttaaatagccaaacattttccactacactaagccattctttgtctgtacattcatccatccacttaatatgcttttgctgagcacccactgggaccagattttaaatgtttctttttttttttctgaatcctaggcttatgcacatgttttcaaactatggctattcttttttttttttttttttttttttttttttaagacagagtctcgctctgtcgctcaggctggagtgcagtggcgtgatctcggctccctgcaaactctgcctcccgggttcacgctattcttctgcctcagcctcctgagtagctggaactacaggcgcccgccaccacgcccggctaatttttttttttttttttttttttttttttttttttttttttttagtagagacggggtttcactgtgttagccaggatggtcttgatctcctgacctcgtgatctgcccaccttggcctcccaaagtgctggaattacaggcgtgagccaccgtgcctggcccaaactatagttattctttaatgaaaaaagaatggagagagtggaagctgatgcctactcataaggcattcaaaaactgggggcaaggccaggcgcggtgctcacgcctgtaatctcatcactttgggacgccgaggagggcagatcacctgaggttaggagttcaaaccagcctggccaacatggtgaaaacccatctctactaaaaatacaaaattagctgagtgtgatggcacgtgcctgtaatcccagctacttgggaggctgaggcaggagaattgcttgaatccgggaggcagaggttgcagtgagctgagattgcaccactgcactctagcctgttaacagggcaagactccgttttaataaaagttaaaaaatttaaaaaaaaaaagaaaaaaaaaatggagacaaggggttgtatgcagtaatccttagtaatgctcctgaccataaatttttatcctgtccttggtccctggaggagtgaaatattctcaaaatttttcagtgttttctatttttctgtctctaatatttatggacactggcttcaacaactttgaattgttataaagaagttgtaggccaggcgcggtggctcacgcctgtaattccagcactttgggaggccaaggcaggcagatcatgaggtcaggagttcgaaaccagcctagccaatacagtgaaaccccatctctactaaaaatacaaaaattagctgggcttggtggtgggtgcctgtaatcccagctactcaggagaatgaggcaggagaatcacttgaacccgggaggcggaggctgcactgaaccgagatggcgccactgcactccagcctcggtgacagagctagactctgtctcaaaaaaaaaaaaaaaatagccaagtgtggtggcgtgcacctgaaatcccagctacttgggaggctgaggcacttgaatcgcttcaacctgggaggcagaggttgtagtgagctcagatcatgccactgtactccagcctgggcaacaaagtgagactctgtctcaaaaaaaaaaaaaaaaaaaaaaatcaatagtctgctgaccttaggaacagcagggtctgagagtgatttttcccagtgggctgtcagtttcagctaaaaactgttgctctaaacaaacaaaacatttggaatatctggagactgaattctgttctcttcattttgagaaggagcaaacatagagctagaacttttattagatggtagaaaaagttttgtcgggtcttgtgcatccttcctctcattccctttgcaaatgcagaattggatccctcacctatgtggacaggattaggtgacaagttaatattgtttcttctaagattgaaccaccattacgaaaaaatcttttcagctttgaaagaattacggctggaggacaacttgctcacccatcttccagagaatttggattccctagtgaatcttaaggttctgacactgatggacaatcccatggaagaacccccaaaagaagtgtgtgctgaaggcaatgaggccatatggaaatacctcaaggaaaacagaaacaggaatataatggcaacaaaggtaaaatcagcccagattcttgataacagttgctttagagggagtctaatggtaaacacagtacagattatcctacggagcaactccttacacccttgtcctgcttctgaaaagagaagttacttggtaacttttatgtccccagggctaagtaagaagatttttaaatgtcagccctagcatttttgtcttttatgtcccttatttctttgtccaataagaagccaacaaataggcaaaggaaaccaaaacttcaacatttccaacaatctaaaattcgtggactatttcattggaatgaaaaaagtttgtattaacaaaagtagtaaaataagtttagaaaattagaaatgaaagcgtttatagatgagaggctgagcccagggttctgagctgccattaataaggatcaaaaggctgagatccttgtaattctgctctagccctttgagcccatggagatgtaggacaggtctggaatgtgtccacttcagccagtgttctttattctccaactggcccaagtccccatcatctcacagccggataactacataagccctctgactactgtccctgcttgcatgccagccccccacaacataccttctgctcagcagttgcactgatgctccactg

SEQ ID NO: 20 - DNA, 3919 bp, Homo sapiens
gacagagtcttgctctgtcgcccaggctggagcgcagtggcatcatctctgctcaccacaacctccgcctcctgggttcaagcgattctcctgcctcagcctcccgagtagctgggactataggtgcacaccaccacgcctggctaattttttgtatttttagtaaagacagggtttcatcgtgttggccaggctggtctcaacctcctgacctcatgttccgcccacctcggcctcccaaagtgctgggattacaggcatgagccaccgcgcccagccccatagcccctttctttaaatgagttggaccaaggtgctgagattagctggcgttaaacctactgtcccccaactatcctcttactccccatctctgccttaggtgcctactcagcaacttctcaccactcacctcctgtaggtagaaaaagaaattcagcagaaaaaaaataacaactaaaacccaaagccccaagtgcaaatgaactcttttaaataggtgtagagaaaggattggtggctcagctgttaaggaacattcaggagagcctgcaacatattttgttccaagtaaatgtctagttcagaggactcagcagataaaatatcctctctctaaaaagaactgaaattactgaaaatatgtgcacatttatatcattacacatggcccaaactgctctggtgagaaagaagggggtacatctgagcgacagggacatgtatgtgtcctcgccccaggccctgagtacatccaccatggctgagggttagatcctcacacatttcagtcccctggctgccagcctacctctgcttgaaaatttatccacgtggttcctcctttggatgctcacaaattgtcaggtgcacactaccacacccagctaattttttttaaagagatgggggtctactatgttgcccagacttgtctcaaactcctgggctcaagtgatcctccagccttagcctcccaagtaactgagattacagatgtgagccaccgcacctggctctttaatttatattagccattttctataatttgcttaattaaaataggaaaagaaataattacttaatgcagtttgttgggggacggctggctcagcgctgagagaggtggccagggaatggtgggagctgcagtggtggtggcagtggctggggtgataagcacagcctggggctgcccctcctcgggggccatgctgcattgcactgcatgggtagccacgagcgagtctaggacgcaacgggaactgtggaaggtgcatccacaaatggaccaatgtgtgtaatgatggaatctccttactaaccattaccactgcctctccttgataagtgagccagagagtgagtgggtcaggggagaagaaaagaggtcaacatgaagctaaagcagcgagtcatgctgttagcaattcctgtcatctttatcttcaccaaagttttcctgattgacaacttagatacatcaactgccaagcagcgtttcactacttgatcccctgagggcttttcaccgaatgatgactggcttgtggatggagctgtcacccaagttggaccacacgttgcagtcttcctaggagactgcagcccagtgggtggttccccaggaagtgtactgtgaagagacaccagagctgggggcaatcatgcatgccatggccaccaagcaaattattaaagctgatgtgggttataaagggacacagctgaaagccttactgatccttgaaagggaacagaaagttgttttcgaacctaagtagtatagccgagactatgtagtggaagggaaccatatgccggttatgatagacacaatgcggaggtagcagcctttcacttggacaggattgtgggtgtcccagagccctgctgatggtgggcagatccgttcatcttcagacagagatcaagcctgttaccatggagcagctgttgagccccttcctaactgtaggacacaatacttgtttttatgggaagtgttattactaccgagaaacagaatcagcttgtgccgatggagacacaatggaaaaatctgtcacactttggcttccagatgtgtggcctctccagaaacactgacacccgtgggccaggacttactgaaaaggcaaattggccaggtgggagtatgatgagcgctactgtgaggctgtgaagaaaacgtccctttatgactctggcctgtgccgcctggacaccattaacacagctgtctttgattacctgattgacaatgctgactgccattactataagaactttcaagataatgagggcgccagtatgctcatccttcttgataacaacaaaagctttgggaaccccttgctggatgaaagaagcattctttccccccctctatcagtgtggcatcactcaggtgtctacctggaacagacggaactacctaaagaatggtgtgctgaagtctaccttaaaatctgccatggcccatgaccccctctccccagtgctctctgatcctcgtctggacgccatggaccagtggctcctgagtgtcctggccaccgtggagcagtgcactgaccagtttgggatggacactgtatggtagaagacacaatgcctttctcccacttgtaactctcaataaaaaataagtgaaacttctttttacaaagatagagaagcagcacaatcaattccaaatggtatgaaatggattggaaatggccagcagcaagttctggtgacaggggacagggtggccttggatgtctttggtgttttctgtagtagaaactaaagcaaagaccacaagtttctgagcatggagatgttcctgctgaatcaccttctgaattcctcagcaattgcccattctagcaataggcatcatagttggtcagtcttaattcccaggccaaaggacaatcagacattttcatagatgaatactgggattggctctggagtgtgtgttttgagtggaacatttcagtcctttctccacaccggtgcatattgtggaaaaatatatgtatacattcatgactaaaatctattagaacacaaggttcccagtacaggagctttccaagaagattctactttttagctggtcctgagtcatgccctgtgaggcatggagcacagagtggaaacaagaccaatgtggttcctcttctcatggcactcatagtccagaagaggggcaccagtgagtgatcccacatccatacgagcacgttgtgaaaggcgggtacagagcatggtgggaacacatatgagggactcttggcctcctaaaggaagaggcctctaaagtgagacctgaaagtcaagaggagaggtgaggcaggagggagataaaggaggaatttccaggtgagggccagagccagagaaaactttaatatggttgggactttgagagtagttcccagtgctacagagggccagggccaaggtgggagaggtggggagagcaagatgagactggagaggtggacagggccaggtcacagagcccagaagacccagtgaaggattgtggctttatcctggaggcagtcaggttttaagtagggagcggcatggttagctttatattgcagaaagatcactcaggttgcttttatgaaaataagttgaagaggacaagacagaggggaagcctattcagataagcaatgatggagttctatccagggtgggagcagtgggggcaggaggataagaacacaacctagaggaggcagggcaagatggccaaaaagaagcctccaccgatggtcctcccttccggaacaccacatggagcaacaatcctcacaaaaaaaaaccaccttcataagaacaaaaaatcagatgatcaatcacagtacctggttttaacttcatgtcactgaaagaggcactgaagggg

SEQ ID NO: 21 - DNA, 3423 bp, Homo sapiens
ataaaaatggacaaagaaacaggagctggcaaaggtgaccttgaaggagagaacaggggtacccaggagggagcagcctcatgacagaaaggaagagcaagcactgttcatgaaggcacagtcaacaatgtcagatgcagccagcaacttcagaaatagaacaactgcaaaatgtctgcatgtttggtgggacagaggcacccggaagcctgaggaaagccggcttaatggaatggtcggggcagagctcagattccctgagactgcagaatgactgggaaattagaacgtgaaagcagggagcatcgaaaccctttggagaaatctagctgtgaagggagtgagagacatgaggcagcatttggaaatggagtttgagtattttcattatttttgtttgtttaatgatggtggagacttatttaaatcccaatgggaaagagccagcagaagaggagagactgaagagaaaagagggaattatctatcctctctggaagtgggaggcatgaggacccacagtatatgtggagagatcgtctttaaatggtggagagacctctcttccattgtatcgtgacagtgggagaaaataaaggtttttggttcattggtcagaattagagggagttcttatctactggtttttgttttctgttttttgttttgttttgttttgttttgtttttttagttaggaagcgctatgatctactgaaaatgagggaaggctctgcctacaaagtctaaagtttgaggagggtggagatagcttgaaatagaagggagagaaagctcaactgtgaatatagaaggatttccaggcaagttgaagttgaagatcatgaaattacggtcctatcaatctgcctagattttgtatccctctatctgcctggaatctttgtccaccagcagccttcctggctttcagagaaaggaaggcacgccacgtccactacagtttcaggactaacagacctgctggaaaggtccggtagggaaagttctcagcagcatttccctcccctccctttaccaagcatctaaggcatggccagcagataccacatctgtaaatgtgagacatgaaatttatccataacaggttttacccacatccctttacttacagcatttactcattcattcatttatccagcaatatttattgtacatcttcttaccatatgccagcctctgtattaggggtacattggtcaaaaaaacactgacctggccctcaaataatcactattcctggttggaattgagttctataaacagagaattacagtcgattacaaaggttattaaagagcacaagcaatatgctactgagcacagaaaagagagcaactaaaaaaaggttttacaaatttttttgtcatcctcaagcagtcttaatgttttctccaaaactaaaatatctaggtggtaaaagaactttttcaaaaaaagacattcagacagagttaacaccttttgtatttatgaaggtatttaaagatcatgcaattgatggagaaactttgccattactcacagaagagcatcttcgaggcactatgggattaaagctagggccggcactaaaaattcagtcacaggtatggtgattttatataactaattatgtattaatggcacaaagcttaatacaaatatgttacttttatcttgttgtaatataaataatacttatttaacttctttattgctttccctctgtggttgaggacataatagaagtgagggaccctgaaaacattggatgggatggtggtacacattcctagatataactgggaagatgataagcaaggatgcactggtaaatgtttaacaagtagttctctttgaaaaaaaatgagccccaatttgtaatatctccaatttctgtggtgtaaattactcccaccgtggctgagaaactcctaatgtcactgaatactgtcatcctccgagaaatcatcatcccactcgggagtgctgccctcctcgctccgggcccatgacacttgggggtgactatcctgaaatgggaaaagatgcatagcagcaccccactttagagtatttctaccagatacaatctcaaatataataggtgtgaataacgtcaagagcagaagtaataataatagtaaaataattaggtaaaaacaattaaaaagtgatgagccttaagcatatattacttttgttgtacataatttatttaatatgatgctttgacttaatttttaataatgatgtatttaacaactggcttgcaaacttcctaaacattgaataattggctcttgtgagttagccaaccccagcacgccacttctcactacctccaccactccatcctggtctcaggcaccatcatctctcattggatgactgtattggccttctttttttttaattttaatttttattttaagttatggggtacatgtgcaggtttgttgcacaggtaaacatgtgccatggtggtttgctgcacctatcaacccatcacctaggtattaagcccagcatgcattagctatttatcctgatgctctccctccccccatcccaacccctgacaggcctcagtgtgtgttgttcccctccctatgtccatgtgttctcattattcagctcccacttataagtgagaacatgcggtgtttggttttctgttcctgcattagtttgctgaggataatggcttccagctccatccatgtccctgcaaaggacatgatctcgttcctttttatggctgcatagtattccatggtgtatatgtaccacattttctttatccagtctatcactgatgggcatttgggttgattccatgtctttgctattgtgaatagtgctgcaatgaacatatgtgtgcatttatctttgtaatagaatgatttatattcctttgggtaaatagccagtaatgggattgctgggtcaaatggtatttctggttctaaatctttcagaaatcatgcattggccttctaactgctctccctgcttctgcccttgcccacccagagttgattcacaaaacagtaaaagtggtccttttaatatgtaagatggattgtgtcagtcttctgctcaagattctacaatagctccccattgccttcagagccaaagccaaagacctatgtcatttgtgctcctcctaacatgccaccaccattaccgtcctgacctcaccatctacttttctccccctcagtaattccactattgctcagctgcccctggttagagccaccttgataccctgttttacatctgtaaacactataattctgagagaaagaaaaagcccaccacatcctacaattgtgacccccacaactatatgttaatagatgctccatgcctgaaattacctcagcctctctgtgatgtgtgtttaatgctg

SEQ ID NO: 22 - DNA, 3234 bp, Homo sapiens
acatcccctcttctggatcctaattcctggagtgatacaatgaacattttttgtccccaggatacaataattcctaaaggaattgagcgaggtagtatgagaaactaaaagccctcaaggaggaagaatagtcttagccagttttccaaagagcctaggatattacaaaggatgtgtgaggatttcccagtactggatggagaatgagaagagaaagtcagcctttctggggctttcatggaggagacttgcccaaggggcttccctgccagggctgaatgaccccagcaccaaatgactggagacgcatccttaggagcaaatgctaatgaaggggagatttacaaacatcaagaaagctgcagatggatgtttccatgaagaaatctctgaagaaaccaaaagagctgaaactgaaaaaagagctccctcccttttttcactcattttccattcccaaaccctgaaagagataaaggcaacccaatgaaaccacagagaggcagaagagaacgggcagaagtacagaacagaaatggagaagctgaccacgactccatctcttctcattccagatttccagcgtgaagcaggcccaagctagggaaggtgagaagctttaaccttaaattttcgagttttggccaggcgcggtggctcacggctgtaatcccaacactttgggagtctgaggcgggcggatcatgaggtcaggagattaagaccagcttggccaacatggtgaaaccccgtctctactaaaaatacaaaaaaaaattagctaggcctggtggtgcgcgaatgtagtcccagctactcgggaggctgaagcaggagaattgcttgaacctgggaagcggaggctacagtgagctgagatcgtgccactgcactccagcctgggtgacagagcaggactctgtctcaaaaaaaaaaaaaaaaatcgagtttgaatattattcatgactaggtattttatttctgaaatacaactatgttatgactgaaagtgactggaggactgctttttatctgagagggaggtgaaaagtcatagtatccacttgagatttcatccaaggggtggggaagttatctctgcaaagcaggtttgagctggcaaggtggagtgctctctgctcacaccctgtggaatcccacattttcaacttgatgtttaacagaatgtctctgaaaggataaacaagaaaatgatgacagtttgctcaaggaggggattggctaacaggatatgaagtcttcttaccatatacattcttgaacattttgaatttttaaccatgagaatatattttctatttaaaaaatttacatttaacatgagcaaactctagaaagcagcctctgagatggcccacaaggatccctgcatcctggtatcccaagcccttgtgtgatctactccgcttgagggcgggctgggggtattgaggcttttctaatgaacagaatacagaaggcgtgatagatgggatgtcacttttgtggcttctgtcttgggtgctcgctctctctctctctctggatttctcactcttggggaagccagattctttgtctccaggcagccctgtggagagccccacattaacttggaaatggatcttctgacgctgccaacagccatgtaaggaaacttgaaggtgcctcagctccagcccacagcttgatggcagctcatgagagaccctcagccagaggcccccagctaagccactctggatttctgacccacagaaactgtaagataataaatgtgtgttgttttcagccactaagttttgaagtaatttatttcacaataatggagtactaaaacaaacaaattaagttgcatttagtaaacacaaattgcatgctacccacattcactttttgatttggaaggtgggcttttgaaatcattttcctataagcctaatttagttaagcttccatttctttcatttttactaagacaacaggtttttcttctgacaaaaactgtatgaaaccatcacatgtatttgtgaattgttactttactctcttggtccctttgctgaaagagagacagggacagacagaaaatcctcccatatttcaaaaatatgtcttttttaagttatttaagatggcaataagccaatgaataacgagtggcaagatttttactaaaaataacttgaaaaatgcttcacttcctctttccctctttttattcatctgatgctcagtgtaccgaagaactgtttggtcacgtgacatttcacctgctatactacccagcaaacagaagtttcacaaaatgccaggtgtgagttacagatgctgtggtgagggataaaaagaaatgcttaatatttttaatggcaaagtgataatgaggttcaatcactatgatgacttttgagtttcgtattgctgctaaataagtccattgagttggtaattgattgatttgcacagatgtatctcaatgttgctctaaggaaaatgttccgatatgccatcttggcatgtactatcaccagaaaaaaccactgtgcttaattttaggtccatgcatttaaaaaagaaccagaagaacacatgtaaaggaggccctccccagctctcagtactcttcattgtaagagaataaaactgaaattgcttgtccctaccaagattaccttctaccaagattgtcttcttctaaagactcctgggtacgttttaacaaaagggacactttatgaacagattattttggagggtgaggatttttatttacattatcatctagaaatagtttttaactttttctgaaatgccacttgaggcttgaagcatactctgtactctttatgaataaaaaacaaagcaacaacaatgacaacttctctcttgttggaaaaccatcacaataaaattatgtttgataaatagggaattatgacttatgaaatatgtttagactgattgagtaaatatccaggcaatggagtactatgcagctgtttcctttagggaagtattatagctattaaggagaaataatagaatttgaatatcaccattatgcacccctaaaacataagcaatctaggcagtagtcataaatggctgctaacatctcgaaaagagaaataaccagacatcatgtgccatctgatggaaatccatgaaaccccatatgaatgaaagtcttattaaaaaaaaaaattaacctgagtcgaaataaacctctag

SEQ ID NO: 23 - DNA, 2048 bp, Homo sapiens
cacaaggatccctgcatcctggtatcccaagcccttgtgtgatctactccgcttgagggcgggctgggggtattgaggcttttctaatgaacagaatacagaaggcgtgatagatgggatgtcacttttgtggcttctgtcttgggtgctcgctctctctctctctctggatttctcactcttggggaagccagattctttgtctccaggcagccctgtggagagccccacattaacttggaaatggatcttctgacgctgccaacagccatgtaaggaaacttgaaggtgcctcagctccagcccacagcttgatggcagctcatgagagaccctcagccagaggcccccagctaagccactctggatttctgacccacagaaactgtaagataataaatgtgtgttgttttcagccactaagttttgaagtaatttatttcacaataatggagtactaaaacaaacaaattaagttgcatttagtaaacacaaattgcatgctacccacattcactttttgatttggaaggtgggcttttgaaatcattttcctataagcctaatttagttaagcttccatttctttcatttttactaagacaacaggtttttcttctgacaaaaactgtatgaaaccatcacatgtatttgtgaattgttactttactctcttggtccctttgctgaaagagagacagggacagacagaaaatcctcccatatttcaaaaatatgtcttttttaagttatttaagatggcaataagccaatgaataacgagtggcaagatttttactaaaaataacttgaaaaatgcttcacttcctctttccctctttttattcatctgatgctcagtgtaccgaagaactgtttggtcacgtgacatttcacctgctatactacccagcaaacagaagtttcacaaaatgccaggtgtgagttacagatgctgtggtgagggataaaaagaaatgcttaatatttttaatggcaaagtgataatgaggttcaatcactatgatgacttttgagtttcgtattgctgctaaataagtccattgagttggtaattgattgatttgcacagatgtatctcaatgttgctctaaggaaaatgttccgatatgccatcttggcatgtactatcaccagaaaaaaccactgtgcttaattttaggtccatgcatttaaaaaagaaccagaagaacacatgtaaaggaggccctccccagctctcagtactcttcattgtaagagaataaaactgaaattgcttgtccctaccaagattaccttctaccaagattgtcttcttctaaagactcctgggtacgttttaacaaaagggacactttatgaacagattattttggagggtgaggatttttatttacattatcatctagaaatagtttttaactttttctgaaatgccacttgaggcttgaagcatactctgtactctttatgaataaaaaacaaagcaacaacaatgacaacttctctcttgttggaaaaccatcacaataaaattatgtttgataaatagggaattatgacttatgaaatatgtttagactgattgagtaaatatccaggcaatggagtactatgcagctgtttcctttagggaagtattatagctattaaggagaaataatagaatttgaatatcaccattatgcacccctaaaacataagcaatctaggcagtagtcataaatggctgctaacatctcgaaaagagaaataaccagacatcatgtgccatctgatggaaatccatgaaaccccatatgaatgaaagtcttattaaaaaaaaaaattaacctgagtcgaaataaacctctagatccaaaaaccaatttacaggaaatacagggaacaaaggaacatgttaaacatcactgcagttataaaaatagtaaatatccaaattatggggaactatagtagaaatgacctgattttctccaacaaataaatgcaagggaatgggaaaaggaaaacctgtagattaaaagaaagatacttaagattca

SEQ ID NO: 24 - DNA, 2056 bp, Homo sapiens
ggtgtggtggctcatgcctgtagttccagctactcaggaggctgaggagggagatcagttgagacgaggagttcggtgatgcagtgagccgagactgcgccactgcactccagcttgggcaatatagagcgaaactcagcatcaaaaaaaaaagaacatctttctattctgaattttcccaattttctacaattaaaaacgctttaaagatctctataaagtcagtcaagggaatttctatctaagcacttaccagcaatttgagctccttctcttttggtctaggtggctctaaaataacaataaaggctacagtgattgaaattcaccagacttgaaatagactgcagccttcttccttcccaccttcttagaatctttgtcctattcatcttccttacgcttccccgggttctcttacttctggattctaacagtgagttgtgagaagcaacgcggaaaacggaatggagactccagcttgttttaaacgagctgtattaccccgcacagtgcaggagaaaagataattggctcaggtctcggctctgccatctaccacctgtggagccttagataagtcagttcttatctctgtcactcaacggtctcggtcttgtcatcttttacatggaatgtcatgaaatatacagtacattgcaggaactgcctcaggtaaagacttctagggttcagtaggtctcagtcgatccctctgagaaaggccgcaccctaaatgcacagactctgactctaaggggatggggtcggggaatctgcattttagtagcttctcaggtgattcagatgctcagtcaggattgagaaaccaggactcttcccagccctcctgttctctgatccacttctctgcttcctccacgtcctaacgattctccagttcccgcacagcaggagcggagggaagaggggagagaaggcaccgctaaggaccgctggccccgccaggcggccgacctccccattccaggggcgccagccggcggattacataacgggcctcggggcgcgttctcgcgaggtttccaccgccccctcctcgctccacgtcagagggaaccgggcggagcggccaacatggcggaacgcaggagacacaagaagcggatccaggtagcaaagccgagctctgggcagggggcttcggcgcgctggatagtggaaggggcggggaaagccccctaaaagggcgagcgaaagcaagggcgaggtggggtggggtggggtggggttccgccgcgaggcagctggctgcaggctgcgcgttgtgaggggcctgagggggggcggtgttggcgacggcggagacagagacggaggtggtggtggagcagcaggggaaggaaggaggcgccgcgctcagaatccggctgcggaggcgcggctgtgggcctggagttcctgtggtcgcgctgtcccgggcctgagctggaaggtcagcctcccctggggcgcagacgctgacccttcccggggctcctcccctcccttcaccccatcctctggccttagctggcctgaaattaaggccacggtaataggggtagcaggaggcggcggactgacctaaagcagctggttctttgtgcgcatagaccgtaagggggaatgatctggacttggtcagctgcctcggggcctaggcagggatctgctcatccagatgttcagttgcgttgcccttctttcccttcaatattattaacttaatagtccacttgaagcttgccggcttttttccgggcgacgtggtgtaaacaaccacaacctcgttggtgctgcaacgagctggtggagcccagaaacctttcacatgccagtgtgtcatcctcgtagtaacgtacataacatctccccctaattccacatgcgtgtcatttgtgcatttcttccttgcgattttccctggttcttgttctttgccaagaggattaattatgtagaacatcttggactcaggatatttattagtcgtctcatcagctgatgtgctgagaaaccttcataagccctcaaattgatcacatgtagtaaa

SEQ ID NO: 25 - DNA, 1369 bp, Homo sapiens
caaattagctacatgaataacaaagatgtaaacaaacatcaaagatgatcactccacacgagcaggcaggtgctgtcacatctcaggacaaatgccctgtttggaggtagaatggagaaagtgtaaagggatctgtctttgggttaacagcaggaaatcagcagaacttcaaagctgataataatcagaagcttgggaacttgaaggactgttcccaagatggaacagcagattcaggtttaggcaacaggagaatgtcactatcagctaaaagcaactggaaaagtggatctccagaagtggagagctgagggaaaatcagaacaaatgtctcaaatctctctcaccccctttaattttcaaaaactataaattaaaacaataagatgacttgaagtgactgccttgaaatctaataagcagatgtattagctgcctttccagaaatgtttgggtggaaaaaaaattatttgaatcgaattttgtgctttgaataaatttaaaggattacacatcaattttagcctttgtaaaaacaatactgtgtaacaaatcactcacaattcagtggctttttaaaaatccaagctcggccgggcacggtggctcacgcctgttatcccagcactttgggaggccaaggcgggcagatcagttgaggtcaggaattcaagaccagcctatccaacacggtgaaaacccatctctactaaaaatacaaaaattagccgggcatggtggtacatgcctgtaatctcagctacttgggaggctgaggcggaagaattgcttgaacctgggaggcggagcttggagtgagccgagatcttgccaccgcactcccgtctgggcaacagagcgagactccgtctcaaaaataaaaataaaaactaaaaacccaacctaaacctccgatttggcaatgaattttagatacaacacaaaagcacaatccacaaaagaaaaaattgttaagctggacttccttaacagtaaaaacttataactaatcgtagtggctcatgcctgtaatcccagcacttgatctctcagaagcaagaggatcacttgagactaggagttcggcaccagcctcgccaacataacaagatccaatctctaccaaaaaaacaaaaacaagcaaacaaaaaactcttatcctctgcaaaagagtatgaaaacacaagccacagaccaggagaaaatatttgctgagcacctctctgataaaggatttgtatttaaaatatacatagaactcacaaaatttaataagatatgaaacaacccaatataaaaatgggcaaaagaaacgaacagatacatcaacaaagaagacacacagatgaaaaaaaaactatgaaaagat

SEQ ID NO: 26 - DNA, 3052 bp, Homo sapiens
aactgcctcactaggtctttgtagaaaaattctacatgctaaggatgtcaacttaaatgtgtctggtttatgaataagaatatttaagaacaaaggctgcatacattaaaagaaataaaattaatataaaagactgagtcaagtaaatgatctgtgtaagttcagaaacttttagattaaatagttgaaaagctgtgaaactagaatgacagcttttatcctttatcatatttaaagcagggtgtctatgcttgacttctgttctcatgctagatataaaaaataagaataaaaagaaagagggaaggaaaaatgtataccatatattgctgcttctaaatcaattacacattgtacacaaattgaatcacctcttacttaaaagctaatcagaaacattactatgcacagaaatgacctgttttccacctaaattctagttcaaacccttccctttgggtctctgaaattttttttattcacttctttttattacttttacatatcaagagtaaatgtggttagttttgaatcatttattttgaaggaaaaaattacatgaaaacaggttaataagactgtgagcattgctatacgaaaaattagaagtcttcttcgcccttttttgataactgcattaagtatatgaacccaaatactttctctaattatacacatcttacaaatatgttcatattaatggaaaacaaaaggtatgctatagctttgtggtagagtggaaataaatgcaccaattatacttaaagtattaaataatgttataatactggcctttgaaactaaaataaatgtcttatttataaagcatcaggaaaacctcttcagctcctgctcaaatacgttgcacatttgaataatgccagtctctgtaggacttgatattggactatgaatatttgacctagaatatcattaacaacttatacattttaaaatcacattttgaaatgcgtcaaaactaagattgtagtgaatttaatttcaaagtcatactatccctccttttatgctgtatgtggtaatccttgatttttttgcactgatgctaaatgttaatgaacagagccaccgtgtaccatttcacctggagcaatgaaaacattaaaatctctttagaagcctgaataaaactttattttataaatataatccatgttaaagctccaccgtttcactgtacttttgaaataagggcacatggaaaatatggaagcgtttcctttgagatagctaactaaagctacaaaatgagcacggtacagcatttccaaggaacatggttcaaaagttgtacgattactactaaggttgttttattctctaccacatttggaacaggaagtctgtgttgttcttgaagaattctgaaaagcaatgtcacactctgatgacctcttctcctcattaaaacgttcccaaagacagcgctctgcatgatgttttgtagcttcagcagccatgaactaatttgtacctcttattttgatcatgcactggtgaaagcaccaaacaatatttttctttaaacacagagaggtctttcaacagctcagcatgcacttctaccctctttttaatcttggagcacctatttatttgctcttttcaccactaaatcttctttgctcctttctgccccctgcaaagtggctagttaaaaatcttcagtgttttttcttcataggcttaaaccattaaaagggtattaccataaaagcagagatgagggcttatagatatagtatgaggagaatttgcatttctaatttttaatccaattaaactaagataatatttatttagttactacctttctataatgtgtatgatcacaaaaactaatacaaaaaaattgctttagaaaattagcttttagggttctctgcaaagactagttcctcacagtgaatattagaagactctttttaaagcttatttgtatgtatataactcatttattttccatacctctacactttctttggttcaattacaccaatcctttctatatgtttctctatatcaccaagaagtatcttgattattgtactcagtgtataggaatttggtttttaagccatgcatttaatttactcagaaaaaacaaaggagagggactacagtaaaatgacattggtaacgaatatgactttttaggcagtaatattatgaactctattaatgaaaacaattcgaagcaatttaataattcaaaagtattgttttaaaatgtatattaattaaagttacataaaactcaaatacgcatatctgctaaaattgattaaattatactagatttcatcaagcctatggtacagctgtaaatagaacatagtgctaagatttcaggtctagttaaggagaaatgtaccattacatactaataaatagattctttcttcatcatgaaatatttattctcacaaaatgtatttactgaacacattggatttcccttaaacagcttgaagttaataatgtaaacatttcactattaataaatcatagtatgttttcaatctcaatagaaaattgaacattaatattaggttatttataatgttaactcattgactttcaaattttgaaaaatcagttgattcttgaatactgtcctaaataaaaatgcaagtcatatgaataatttctcaatttatttgtaaagctaaacatttgtaagaaaagaaatgaaataagagaaaggtgtcatttatgatgatattatcagtatttaattttagtgtcatttaagattttttcaaagtaaaattaattcaattaatgttatcaaaataaattatgtgactttttaaagcaccatatctaattcattgatccctttcactattccaaatctcttctcaatgaagtaattttcaatggcaaactggaatgtgtaaaaactgttaatttcatataatattcctccaaatttaatcaatatttgctgtttaaagttgtttcttctataaatttgtctttggctaatttaatcctaaaactttaccacatgttctgttgtgtctct

SEQ ID NO: 27 - DNA, 3984 bp, Homo sapiens
cttatcacccactgacagcttctatgtgaatatgattcaaaccaaaacaatgcaatcctgtccttcccattcctcacataaaagtaataacaaaactgcaaacaggtgtttttcttcttttgcccatatcctcagcattttagtagggacttattatatcaagggtacccagagccttgctagcctttccctcagcatggttctgtgctgtaagagataaataaatttttgaaaattaaaacatcttaaaatacctcacattgtgttttcccttgtattaatcctactttaaaaaaaaaaagccattcctttcaccattaaggataaggcataaggttcattgtcctagaaaaagagagagagtggtaagagtctagaacaaacaggcttctcattgtagctcagagtttaaatatttgagttcacagtcagggaatactcttgaaaatcaggctttgtttagaaagtaaagaatgttctaacccaaagccaatctttacataacaaggttggagatctgaatataaaaattccctataagatataggcagagaacattcagcagatcacatatgctcatatgtgcctgaagctcttatctgcctacaggcttgggatgcacaattgggaagaagggtgttgggggtgaggagggggaatgtgcaggaactggggaaagaagttcagataagacaagtccagggtaaggaagcagcaggcacctggaaaggggatcaatccagtagttgatagaagttctgacacaaactccaaataagttgagaacaaacctgagaacaattgaatgggtgctccacttcacaccttggggaacagtagaacagcaagagcagataaacaagtcccgtcacatggagcagggtagtcctacatcttctgcctgtctcctgaggaacaagaaatgatacagaattgcccatgtatgggagaaggcagatagcatgagcttacctggggagtctggataaggagaaagttaatttacatacagcacaaagctttgggatcaacttcccatgtcagggattgaatgagagtcacagctatatctttgggcaagttctactaagccagaaatagtgaaccagatggtggaggtgacttgatcagataacagaggtgaccaagttccccagagcaatctcctcaccatgtcaaggccaagagaacagaccacaaattgtaccacctaaagacacattaaaagtggcagcagtatggagtcctcaaccatggccccagtgaactgcatgttacacagttatgtgtcatcaaaagactgatatgcctttatattatcagagtgagatttgatgccaaaacatccaagccaagttctgtgggagtcgcagatagagacaaagcacaaagcaaattgttccccctcagccttgccgtgggttttgtaagaggcaggagaagagaatatgtgaaagaatgagcttaacaaaagagaccattttaaaacagaagagcatgagctacttaactgacaattttatatctattccacaatccccactggtaaagacgggggtttgtaagcaaaatgatttcaaaataaagaaaataaactggttactttgttacttttttttcttgcacatctgaatggtaatgcacatattttcacctcccatcgccaatcctaggcatttgctcttgggaaaggtaccttagatcataagtcagctaataaagagaatggcagacccatgttctcatagtgaaaggattaaaaagaaagccctgcaaggaggatctacgttcactgtttcttagagggctgaagaagagcatcaacgtccaccagttcgaatcccagaatctcagactcaggtggatccaaggagtgatgatccttcctgtgaaaagcagtagaggttttgcaagggaagatagtagagtattgaatggagaatgttcaggaatggaatgtttggagctggagggcacacaggggtttaaaggtgaggactggctgcgcatctttggaaggtggaatgggagtcatccatgacgtcccgttcttaggaagccctacgttattttggttccttaacttgtgagatttgaggaaaactccaggttaactaggactttaatatggtcccttttaaccttgtaattcctttctaaaatttctctcatcctaaaataaagaggtcacatatttttagaaaatcaggtgtgatgcctttgtggaggatgaaggatgaaagtagacaagaggtctcatggatccaatggaaaaaagtatgtaataatattggggcaaccacacctcagaataggtgaatggatgaatagaaatgccagagcatgacccacagagaaactggtaatggagggagatcctggaatctggaaaatatcccagaaaaaatatgtggatgagacattttacaaccaaaccttcctccaaccctcacaggttactgaaggaacagaacagagcttgtaaactgcaagctacatagttatgtgtcttcaaaatactaatgttgtttgtattataagtgggattatgagtcattttgtttcttctgttttctagagttcatgtagacacatgctttccatccaataaatatttatcaacatttacaatacaccaggcattcttcatcagtgagtaaaagaggcaaaaataaacccctgcctgtactctaaagaaatacaatgtaaaagcagacaaaagcaactattaatatttttagctcagtggtcaaatatgcctctctcatgtgtgcacaaatgcatgcacgcagaggcacgcacacatgcacacaaacagagtgtctttcccggttttcttaatttcaacctgtttatttccccagtctccccacttatttctaattcttcatttcactcataggtttattagaatttctcagagttctttgagctaaaaagctcagtaattataagtcatctgttgtgccccgaaacctttattaaattttatttagacaaaattatagaaaacaattcagtatgcttcaattaaaattagccaaactattttctcatacacactgcattcctactgcctcaacacacatatacacaaccacatacaccagtaatattaaaagttaaaaagtaatttgcaattcaagtatgtttgttgaaataattactacagttgtatatgaatgttcagtgaggtaagaggaaattgtttattactgaataatgaaaaaatattacgcttgtccattgggtggcagcaaaactcttccatactgttaaattctcaaagcctttgtcttcagtgtcttcatgctaaatttgatagattatactacacacagaggaaaatcctcaattcttccctttcaggaaggggctgcagaagcagtgagattagtgctgcattgagtttaaagtcttactgggtatagaagaaattatcttttgaatttaaaaaacaacaaattttattattttattcttcaattcaatccaaaagattcgtgtcaattacgtgtcagtactatgctggaaactttctattttcatgaagttggctccagcgggtagaactctacacgaaaaaataaacatctgcaacatcaaaaacacccggcttcaatctccatcttccctctcaacaccctcctatcatctgagcaaacactccttctttccaaaaagttactacttgattggaaacttaagctagaaaatcaccataacacagcatcaaaattactgtttatctaaaattgtataagtaaagcaaggatatccactccatccctaaggtcagaatttttaaatccacaaacaaatatttacttaacctgcctgctccacaggggcctttgaccctccctcctttgctcccagaacaaagagagaaaagagcaatacaagtgaaaggaagagaattaacagttgagtgtcaagagatgctgactgaaatgaacagaacagggcaagtatgttaaagtg

SEQ ID NO: 28 - DNA, 1726 bp, Homo sapiens
ctaacttaattctgaatccccttccctttttttccccatagaacttttcttaacatcctatggagaatctggagagagaactgctcaataggacagtttagaaaatttagaggtttgaaaactgtgttaaatatgggaactgatttatatactctcttaaaactatcaactcagtatagcacaaataaataaacaagctgtttaaatctatatatatcatcagaagttacttatggatcatctgatgaaagataatccacacaccgtgcaaacagccccttgcacatgggtattgtgctcacccatactctgtttgtgtttttgttttaggtttgtataaaaccacgtttttaaagttcttcattttcttaagggtagaatctgggttagtttaggctgaaattgaatatgtgataaacgtcccacttattctcaggccgctcttggataacaagatcacagtggcatccttttcttctccaattccacaacaaggtttttcttttaataaatgtattggattgcttccaaatgactttgaaagtttttataggtatttctaaaagtaaaatgttttagtttaaatgtgggagactcaaatataaaacactgtagattagaactctaagaaaaatatttttaaaacctaaaactaggtaagtggtaatagcacagatttccacaatagatagctcctggcacctggtgctgattttatagtatgttttcttgcagagtagtttgtagctggctcttctccttgtagaaatggagctccagtgtaaggcttctggaagggatgtggtcatcccaactgttttaatgatgacagctctagttgaagagccatcccacccttcttgggtcacattcttaccaaggactctgtcaccacaattcaccacattggaggatgcagatgaggcttttaatatagtcatgatttttaaaattataacaaatcaatcataggttttctctacttaggaaactgaaaattatatggcatagtatgacttcttaggtatttcttttttcttaccactagcgttattggtatgttgttttaacctgattttactcacttattaacaaaggtatgttgggtggttaaaggcattgatagaaaagcagaggcttcctgagattgtgaattcttctaattgtcaagacaaataataatttaaaaaaatacagagctggttcttctataattagtaaatttaactctttatatttaacactttcacagtaaatataacttttttaaccaagccacagctcaccatagtttattcatgcttcagaaatcatcaggctttatttcacttgtatcttttattatttatctttcagattctaaaatctgacccttattttgtggtattcatgttgtactggtgtagaacatagattgtaaaaatttccaaagtccagtgtttattatacggcctcgatcctttaagataccactttgctaattattgggatgcatgctgtagctcacagccatataaggatctatttagctatattaagaactaaattcccactccaccttcttcccccctcaggaaggtataattttaaacttgaaccaaaagcagtgtgatagttttataaataattacattgtttttgttaaccaaagagtaaaagtaaagaacatgctgtagtaggataacttgctgctttccactagatgggagtatgagataaccagatg

SEQ ID NO: 29 - DNA, 2343 bp, Homo sapiens
aaaatatgtcgtaactgcaagtgtggccaagaagagcatgatgtcctcttgagcaatgaagaggatcgaaaagtgggaaaactttttgaagacaccaagtataccactctgattgcaaaactaaagtcagatggaattcccatgtataaacgcaatgttatgatattgacgaatccagttgctgccaagaagaatgtctccatcaatacagttacctatgagtgggctcctcctgtccagaatcaagcattggtaaatgatatggaacagagagtttctttattgaagttcctggagtatttatttggggcagtttctttgatgacctaatcaacttcacacatcccagatctgttattcttccggggttctcattactttgacttctgattcctgttctagtcttttgaatgtaagactagatacttcataaaatttttccaactccctagatatgtttacctttttgagggaatggtctaacgttcttaataattagatggaaatcagatgttttatatcaatctagtaactcttctagcttagtcttgagaggatttatcttaagatggtaaagaagagaatttttgtcatagtttagaactgaaaaaaatattcaacattatctttgtgtttttttcatttcctttattaatactatataagaccaaaaagatcacagttttatatcaagctccttccactattataacaaccaaatgaaacatttggaagtcttttaaggttagttaaatcattaaagtgaattttgcgatatcattttttatgtaataaaaatgttcctggtcctacagaccattccaaacatgtctgcctaaaagtttatgttttctttcaattgtacatttgtttatttagcttaagtaaacagtagtaactcagatcttacaaactgaaaaaatatataatttcatggagagaaagtgttactttttacccaactaaatctatttagtgtaccgtttttgagttttaacttcttaatatactgtgagggtgagttgctgctattggatagaacccacttctttcttaagtaacttgcttctgatgtgtgttatgaaacatcacactgcctaatggccaatcccagttaacattggcctccttgtggttcaggccaggcagtacatgcagatgctacccaaggaaaagcagccagtagcaggctcagagggggcacagtaccggaagaagcagctggcaaagcagctccctgcacatgaccaggacccttcaaagtgccatgagttgtctcccagagaggtgaaggagatggagcagtttgtgaagaaatataagagcgaagctctgggagtaggagatgtcaaacttccctgtgagatggatgcccaaggccccaaacaaatgaacattcctggaggggatagaagcaccccagcagcagtgggggccatggaggacaaatctgctgagcacaaaagaactcaatatgtaagtagagtggtcacactgttagcctgatttgtatactttacagaatttttttcccttactttgaacttttttggggactattcctctgagtttccaaaagggaataagtatggggaataaataggatttagattcagtttagacttcccacagagggtcatcgtgtttgaaacttctgtctaaagccaaattttctatgtatttattatcaaggataattgtggatagtgagttttatgtacagacagtagttggtattggcatgctgatcaaagacagcattattattctatagtctgtttctaatcataatgttggtattaattgcttatctctgctgcccataaataggaatatatgcttaggttaattcagttttgcttgtgagaacgtgcctaaaattcaaacttaaatttaatttttgtggatatatagtgaagcttttttttaagtctcttctaaagggagaaattgaagtattttgctaatgacttgatggtaggaggcaattatgacacagatgaacagacataagaactaaacctgctgtaatatattcctcatagctccagttagcacaccaaccaataaggcttagtcctgaaaaaagagcactaataagccctgaggtgatagttttagagaatctacttaaaattgacctgaaaattactattttcctttttccataagtgaccttaattcaggaaataatgcctgttacacacagggtgaatgaagtagatttgaatagccgggagaaagctgagattccatttcacagaattattaatacgttattatctacatcgacaaatttgtaatttcagtagaaattatgtctcttcaaagttgtttctctga

SEQ ID NO: 30 - DNA, 2163 bp, Homo sapiens
ttgcatggtttttattctttcctaatggatcctgttttataatacttccaagcctgtccatggatatatcaaatgtcttcacttgtatattttcatggctaggtatttctaatgtttattcttccctgtgtacttctacacatagctatgcactatgaaaattaaatggaatgaatgatatgtatattactcaaaataaagtttctttcactttaataatcattgtatctttttattcatatccttttttgagtgaaaaatgcttctgcaactaagcatgtgttacagcagggggcgatgtctccaaggataaaaattacccaaacgaaaataaaagtctgcctctgacaggacagcacagaatctagatgtttcattatggagaaaagataaggtttatgaaactttcaaatcaggacttttaagtttggtcatgcaagagtttttttctttttcaaagaaagggaaagtaattttaattaaaatttgggagagcttattttctattcagttggatctaacaagccttttatacttgggtttcaactctggtttctttatatgtaaaaaatattaaaagaagaattttttcatggaaacttatcgtgagacagaaagcaaatggggaatagtggtagactcatgcttcctaatgttagcaggccataatactttctccctcagtctacaattatttctgtcatagtgggagttgcagttttctctgatacatacccagaataacttgacttttttttttttccagctcctgtttggccaatagtacttctctttacattccacctctccactccccaccaccacacacacatgcacatgtgcataccaagcatacaaatttactccttctgcactatgccttcaaccatgcaaaagtaacttttggagcttggatacgtgtcaccttatctaaagtaatctaattctgcatataattttagaagctaatatcacaagtatcattactgtctataaagatcgctttatggttgctcatatctttatcttaaatggcaacaaatatattttccacagcatattgtccacaaagttgtacaacttaacttttcccagacatggctaagtgagaataaactgaaacagaacccatctgctactaacacggccaatcccttaggaaatctggattgctcttgagacatactagccttcatatttgttgcatggtacctacatatctccctttcctccttcctttttttttttttttttttttttgaggtggggggggcggcaaatgagttagggattattctttcttttcattgactagctgaaacttctattactgaagtcttcaagcatagatttccaaatttgactttgaagtatttcagcccaatttcaccaaaggcaagggattcatcactgattcattctcaaggaatttccaacacatagcgtttaagcttatacaacttcaccgtagttaccaaagagacaccaacacccaagatcagtattcagcctttgtcttgagatgccttggcatatctgaacttgtcttctgggtattcacttaattctttggaaaacttgggtttccgtcaatgtctctataaaaaggcactttgccacaatttctcactcatctgccccttcttgacctctgctgatttaaatgcttgccgacaactttgatgactgtaatgaatgcccacatgtatgcaaaggacccactgtgctggtttatgtgtatgtgctttgaccacacaaaacacattctggctcgattaatagattaataggatcttttttcatatatttagttgatgcttaaatagctttggctaattattttgttctttcacagagaacttgtcattgtagcttttatttgatctgataatacaacttgaagcactcaaaataagatgccattaactttttatacttgttttgaatggcatcttatgatgacatttacgaaggctgctatcaattttttttcagtgatactcctcaccagggtatcttgcccctcagccaataataaaaccaacttgtatcctgttatctttacgagagtcagacattagtgaatacagagactaatattacagttgatttctccataaatattgatggactgtgaagacatcatggtatatagttccaggg

SEQ ID NO: 31 - DNA, 2255 bp, Homo sapiens
tatgaactatgtcaggtcacgatgtcagcaccacatagaaatagttttcttttgcttgcttgccctttcacagagatttcttccatgtgcctttgcagcttccaaaagctgtagcctgttctttgagaaagctcttctctccttaaggagttgctccacatcggattgattctgggccatgcatcatgtcagcttgcaaaagacaaaaccctttctgtttctaaagaaatttctcatattttgaaatgaaagcaagaattgctctgccgaggtgccaagggcaacatacttccattacgaagtgtttctaacaaaatgttttggagattttaaaggggcagctttctaaatatcccaggcatctgcaaacaaccagacatgcaggcccgcttttcagcccaaacattgtttctgggttgtatcatttttttttcctgaaagtcagcacagctgttgcaatgcccctttatcaattgtccaatgatcccaacaattttttttccaagagtgaataatcaaaatggtattacaggtctttgattttgaaatatatgcactgttaatataattttggccttctttagcagaatttcaaaggggaaccaaaggtttatatttataaagtttataatcattccacaaagttcaagacatttggcagtgcttgggaaacttaaaaacagtaataaaaggttactgcgaggaattcaaagctgccttgtatggtaatgctgattcaagccaccacatactgcagcattagaagtttagctgtgatttgggggttggatcagaactatgcagctgtagagatatttaaatggcttacccattaacaacaaagaaagcactttggaaaatttacaattaatatctccgctttgaaaacacattaggagtatgcggggggagaaaatcatgcattcttagcctgatttactaagcatctatcagcctccttgactgttctcaagcaccaaccttgccttgcaattgtaaactgtgcacttttgccaacctggaatgcacactctgtccctgcccagctccagatcattctgcatctgtgcttgctcaaactctggtaacccaagctcttcatttgttggcagctggtaaaaggggtttccttatgagtcacagtgtggagggaggctgagtaatataccctatcctgtgctacaatctgtatggaatgtatttgaaataaaactaaacttagagtttagttcgacatctgatttcaaacacattcttgaaaaattggagtgcaatagggaaaaaatcaccatgtctctcctgctacggaaagcttactcccctacaactgtctccaaagggacagaaactaaattgcactcattcaggctctgaggtctctgatgaggaaagagtccacagcttggcttttttagatgatcctgctttcctgttatggcaatgaagtagctagtcccatggcgaaggaaagtgggaaaatgaagttgacaatgcacaagtgtcacaggctaaggcttttgtcaatgttgtttaacaaatgtaagcatctgacagatcacatctttaactgaaagaaggtaaagaatattacaagacaaagtgtgtttacaaaagtcatagatgatgcaagaacactgtttatcaccatatattcacctgtttacagtaagttcaggaaaatgggttttcttactattaaatgcagtaacatgcatttatctagaaaaataaattaagactatcaaggtctaaatataatttcaaaaaaactagacacactgtactaaccaaatattatatttttttctttttaaatatcattagatacccaaatatctgaaatgaaaaaaagattagtgacgacatactgaatgaacggaaaaactggaaaatttgtggctagaaatttagaattatactataagctagatgtatcaaactcacatattcttccaaattagagaattagttatgcttcagaaatataaaatacatatatgccagaaagtttgctctgtttactaagcaataaaataatcatatcttcttaattaatgaatcttataaagtaaatgaaatggaaatatgtctgttacctgctgcttttgactaatattcaaataaaataatcaagttatttccttcttataatttgtaacacacatttctaaatacaggtgagttgcatgagaaaaggggattgaaaaataaaaatttctatttggttacttgtaatttgggcttgatacaaaaat

SEQ ID NO: 32 - DNA, 6897 bp, Artificial
  Bacteria Artificial Chromosomes from Human Genomic DNA
cttgtgtaactttaaaatctccactttacattttcattctttgattttaataatatttgaaaaatcttttgttagctgcttgagatcctctccctagtatattaaacttttaaaaattaatgtctctttggaagttattacttagaaaaatcagattgaataaacatttagatactacataacttctttttctttcggaaccttccattttttttactgctatgaaaattggttatcctctgtattttgcaggcagtttcccactgtcatgctgtaatcaggaaaaaatacaataccttgttatagatgtccagtattatattgagagatatattatggtatacacagagcctgttttttgacctttagcataagtaaatccattgaacagtgtagttactttatgaggtcttacattcattaatgatgtattgtggtacttactgattcacagttgcactttaaagtcacaattaaatactaaatggattgtcccatatatcattaaatattttttctttcttgaatttcttaggtacaatgttagctgttctaaaaaatttgcacttagtcgttggaatcgtaagcctgataatcaaagtcttgggcatcgtggccgtcgtccaagtggccctgatggggcagcgagagaacatatccttaggcaggtctgtataaaccttgcttcaatgtaaaatacccttttcagaaatgcaaggaaagtgcaatgctgtattttgcttgtactacattttccagcttccaattacttatccatctgcagaagaagacttggcttctcatgaagattctgtgccatctgctatgacaactcgtctgcgcaggcagagcgagcgggaaagagaacgtgagcttcgggatgtgagaattcggaaaatgcctgagaacagtgacttgctaccagttgcacaaacagagccatctatatggacagttgatgatgtctgggccttcatccattctttgcctggtatgtaattcatcactttggccttaatatttttcttgtgcattcacacacagcaatgttagatacatccacaccttttgcacaaaagggctagagaagtactgtgtatatgtataaataaaatgacatttgttattgaaatatgatctgtttcctcttgcttattccctcagcactactgggtattttctcataccccccaagtcgataaaaactgactagtcctgcttctgctgtgacacctcagccggtgaagatattaaaggtgctttcttctcttcagtctgatttctctcgagaggtcagtagagagattgaatcagacacttcttcctttctgctcttctatttttctattctattcctccttatttcctaatttaaccttttaaaaaatgaagtaagagagacttttttcttttttttacatttttgaaagctaatcaaaaccttttaaaatgtaaaaatttggtttctttgaaaaaatcaagctagtccttttaaaagaaggtacattctttgattcttttcaataaataaacatcaagagcctttctgaaattttatttttcaaatagagagctgaaattcaattatattcattctaatattgagggtaactttcttatttatgttttcctgttctctcattttgatactctttccatcaccttaagtctctatcttttttccttaaaacaacaataatcttttatatccaccttctccaaggggctggctactaaagacagctgcacctttgctcatgatgagacccacactagttatatatgttaagactatacatatatgtatatatatacttttttaaatcagtaaatcttgtcattcactttgaccttacgcatatggaaaatgaggagactaaaacaaaaaatttatttccttttggttttattgcgaatcatatcatttctcttgatacaagctttctagaagaatacatcctatttgaacttgtctactaactttttacagtttaataagctaattaatcagctgttgttgctaattagtctttctctgatggcaactaaagtcttggaagcatacttagagctaacatgctatacaagaaaatatatttaggaatgatagttgtaaagaatccgtcacccactttacctcatatacagaattctgtgagattttgttaaattagtttatcaatagcttgttttagaaatgctaagttttaaaaaaatatggccacttttaaagaaagatggaattgtttaaagtggagatacttcatgggaagatttttaaacatgtgcatgtgcccacgaaaacttagatgtgagttaattgctgaccatgtggatttagaatctgttttaaactgggtgtatttagaagacagtgggtcttcattggttgtggttttagtatctaatgaaaccagttttagtttttagaccagtatagctatcttgagaattaactttggagtctatgtggatacctggggctgggctttgattctgtcatcttcagaagtagtgtcactcttccaggtagggcacatgatctgtttgtgagcctgatttttcatttgggggaaatttttcttttcctttgcagaagagagttggtttatatcctgagaaaagtagttgcccaaataattaacaaatatggatactgctgattcttctttcacttctttttttatttaggctaattttccccagcatcctttgccatgtggctactttaccagaacaaaggcaaattcctttgcctcattcttgcatcatttgagttaaaaaaacataaaacttaatattcctcataacttaaaatgcttgtcttatagagttcatctagacacttaattttgttgccaggtatttagaaaacattatatatcataaaaaggacacagctattatataatttagaattgcttactactattttagagtctggttaatatgactctcttctttacttatgaaatgggagtgtttccaaactgctttgacaggccaaataataagcttatttgtgtgattagttgtatcacctttctcacagttttctctgcataatgcctatagtttaaggtaatctcataccagaaaatgccttagtgttcatttctttctgcttctctggactttgtaaatttcgttttgagcttcttttagctctcattaagcgctgtttgttttccctgagtagtctactcccaagcttttctcataggatgaacaagcttgtaaattttgtaatctcttttgtattttgtttctttccagttgagataggcttccttccctctttaaccagggcatttatatttactcctccagttttctcttctctttgtatatcgtcttgttaaccatcctgttcagactatgttagagcttgtatgcagtagaccagaatctcagagctgttaacagaatgaaaaattaccgccttctaattttctctgcattaaaaagttatctacaggaataattcttattttcaatttactgcgggttatcttctttaacctttccttgctatggtgaactgccttttgcagagatcagtttgtcctctgtttattgaagggtgaatccacagtagatttgatttttctgtaggcatataaactaggaatcattttttaaaacatgactatttactggtttttgtaatatcagtattgggacttgtttattctgtagtattgagtggcaggtgataccttttcaccaaccttactgtggtcaatttttgtgcagggtgttttattttgagtttagctggagaagtgatgccagacacttgaaagatcagttgggacccatgtgcagaggtccttgcctgtgctgagagctgcactacacttgcttctggtgaggtgaagctagctcagctgactggctgtgtcagttggtgtatgttttgtacacatccccgggatccaggcattttaccaaagtagatgttaatgtcagaaactcagaactttatctcagtataccttgagcacttaaaattgtttttaaaatatatttttctgtaatgagattctggaagcatacttgcttccaatttttctaagtcacttttttttaaatttctatactgatgtaactatactttttttgtggttagcttaagaaagttgtgatctaaaatgcattcctatttgatcgtgctaattcccaaaacactttttgttctatattattttgctaatacatttactgttcacctacaagaggatatttgaagaggaatctcacaatacagaggaacaggaatgtgctacatattttcctcatggagaactccttgtttctattcccaaatgtagctcttatagttctcattacttctcattgctcttgttttctgaatctaagtaagtcccaccctatctttctctctcttcttttctttcctgtgttcttgtaagtaagactgaagagagaagaacattagttctttcataaaaagaaggatgaagtcacaactagatgcaagattagtcagttttgtctgcccagttttgttgagagctacctctgggaaaagaaaaggggcttttctatggtgagtaatcccatttcaactatagtttcttttataagataaatgtgcaaaagcataataatttcgtagagggtattgaaatgtcaatttttcaaagtctgagaaattattaaaatagaaattgaaaagctagttgttatataggaaacagtaaagatacaagtaacagaggacgtgagaaaagaaataagcaagagaaaaaatggacacacagctgtacacaggaaaaaatgtttgtattactcttacagatagctcttctatagcagattatatttttgtttattgtgtattcactaattcagtccttattgacaaatattatgtattcagtacctataatgctttgtgctttgtgctggggttgtaagacatcataatcttacataaacaagaggatgatacatttgcaagtagaaaggataattgaactagcaaaattgtggcatccaaaagtgtaatgtaactattttcatttttcattatctttttctttgtgttttcccttagaatgctgatttgaataaaatagaaaaacttaagctgtcagtttttatgtgtattcttaaattcatcttaaaattttactgtctctcaagaaagcttccatatttttatattctctcatctcagctacttaattgattacctttcctattggtttctttacccttaatgggtaagtttattgagtaatttggatgtgaaaatttggtagacagtaaaaacatttgagtttcaggtagatactagcattgttacctgctaaagcacctggtttagactgagagctaaaatattggttatatggcaggaaattcgccagcaagctcataatttcaatgagaattgttttcttaacaaaagtggagacaagcccagtgatagtcaagtcaagcagcgtaatacatttcagcagcattgcaatagattggtaatacagtcttgagaaatttgatatgtcccgagtgaacaataaaaatattttaaatgtacttttcaagtgaaggtcataaattaggataaggtacaaatgataatgaaagtggtacttacttgagaaaatatttctagattgttatttaaatgacaaaacatatttttgtttcctctttacctatatgtgaaggatatattatacagagataagtgcatgaggtataactagttaacccaaacctgttatgtggcatgctttgaggtaacaccatagtcctttttgtgaaggaagtattatatctagacttccctcttatgaattatttctataacattattttagagcaagaattccaagcaaattgctttacctggattttgaattcttattttcagaatgcaaatactctattcttcaagagtgaactcaagactaccataaataaatttcatttgtttctcagcctgtatttcagattctcagacccagtgagagcataactgtgactgttaggagtaatgtctaattgttgtatatttaattctaacttctgtcctctggtcaaattccaggctgccaggatatcgcagatgaattcagagcacaggagattgatggacaggcccttctcttgctgaaagaagaccatctcatgagtgcaatgaatatcaagctaggcccagccctgaagatctgtgcacgcatcaactctctgaaggaatcttaacaggaacatgaagccttgataaaacagcagttttacttttctcacaaaaacttgtaaggtaaaggcctaacttggtctagaatatgacacttattgtggtggatagccaagcacattgggatctccacatcaaatactgacatttcttctacaggtataataattcatcatgcattttcataattaataaacattggtaaaattaattttacaggttacatgaaacattgaaagacttgttacagagggccatgatatttttcaaagaaatgtgttatactagataatttttttaaaggtgatgtttatcattaatataaagaatccttttaaaagtaatttaatgatttacatttctcctcttttgattcaattttcttatacattttttctaccctattagttttctaaaggttgtcatgagaggtatattatggaataatttagtagtccagtgacagaatcgtatgaaatcagtgtacattttaaaaaacatgtcttttagacatatgctttatctataaaaaaggaattgtgttctagtatgaacaatactgatctggaagtgagaagagttagtttctattccaaacttgaccaagaatttggtttgactgagaacgttttcctctcagtttttgtacatttatttagagc

SEQ ID NO: 33 - DNA, 25 bp, Artificial
  Bacteria Artifical Chromosomes from Human Genomic DNA
gcttcaatgtaaaatacccttttca

SEQ ID NO: 34 - DNA, 25 bp, Artificial
  Bacteria Artificial Chromosomes from Human Genomic DNA
ctgatttcatacgattctgtcactg

SEQ ID NO: 35 - DNA, 4044 bp, Artificial
  Bacteria Artificial Chromosomes from Human Genomic DNA
tatagaaggagtgataaatattgttttcacccaaaggaatacttttaaaggatgaagcttactaaacatatatgatggaagtattattcagataacattaatattctgctgaataattttttctagtttaatcatactagaaaaagaaaaaaaatctacaaattgtcctataaaataaggacaaacatgcaaataatttaactctcagaaagtactaattcattctgattatctttcatacctctgtgctcctctgcactgacgaagacataatatgattatacctatgaactagtgcacagccttttctggcaagaaaatagtttgtagcagatacgtggttgctctttggatttttttctattgttgaacatgctgggactagctagaatgcacattcctacttcctttaccaaacgtttgcatgcttcctgcaaagcacttaccaagtgatttctcttgaaccatcggatataattttgtatgtacatgtttgaggaaaaaaatgtaaagcaaaaccttttactgaacagtgttctatagaattatgacactaaaacaaaattgtttgtggaagccctgaaagctttatagtcctggacatcaaaaattttatttgagatgatgaatgttttgttttcatcttttcttatattaccacaattgagatattttagtaattgaaggaacatacacagatatttggcagaagtcgagtaaggaggggaaaaaaagagtccgtgagtttcagtcattttcactgctcttttcaaaaagattgtgttgagctggtagaagactaaagatgtcactgaagacatcacagatactatatttatcttttggctttgtgtacattagagaatgttgattatttttatacaaaaatacagcgggtaatttttttaatctttagatgcctcttgtttgaatgtatgctttgtggaattctttgtgtagtaatgttttaaaaaaagatgtttactgatagttacatgtaggattagaatatgtaatataatataaggctcatgttccagacctacgatagcttgtagtctatgttacgtatttctttatatcacatttttaatcattggattaaagtatcaaggaaagctaggtactctataatgagttttcatttattagcagttaatcatcatgacagaattgtcatatgcttgacttttccctcttcttggaatttcagaacacaaatacaggctaagcattagtaagagatggcccacagtatgagagagagaggtgcaacggaaaatctcgcctggaattaaaacttttcatagattatccacggttaatacaaaatttattatatggggatagactgctccagcaataatgattacatcctataactgtattacctatggcctttaaggtatcaattttgaactgtgttgtaggctctccttttatttgttctctttcctaatagcagccattctgtacttattgaaagcccctgtgcctactgctgtcttaagtattcaggaggggcttacaagagggttttctattggagaataccgtataatcttaaatctagtccagatctctgttgtccccactcaaaacatacacaaaatatgcacttgcttttttcaagtgagtttttatttaaaaatggcttgtttgctatcacattggtgcagctgtttctttcaagatgagttaatcatcttaatttcaaagcttcagctatatataatggatatatagacaacactgagcatccacctctctcctgagctttaaagcagagtttcagtatgatataggtggggagagtaaattgttttcatatcctttcatactactactaatagttttaggattttgactggggagagataatgacaaacagaaagggaacatggaggttcttcctacttttgctacctaagtttgcattttctgacttccttgcagtgttgcactctttgtcccattgggataaaaagcataagtttgaaattttgctttaagccttgtgttcctggggaagttaaacaactaagagagctgatttgtaaaaattattttttatatgacattaatattcatcaagccttgtgtaggcatgtgtaagacacagctatgcagctttgagtagtcaatatagtatgagatagagtgttgtcccaaatcctcctgtcactttttaagtagcatattatttccctgatggtcctgttactttgctgttgaatgctctaaacagaactttttaaaaggtgtgttttaagagcagtcacctaggagtagacaaggtggaatgggaggagagaaatggtaatgcaaaagcttgagcatgggaagagtcagaggaggaggccatcatccttgttagcttagcctacttcaacactgagcacatttctgcacttttgaagtgaaattcatgttttacttagaagaaataattttctttcattagggatcccagttgatttttgtttcctggtgtatcaaaatacttagaactatgaaacaagtattattgtgatcatgcctttgaataatttttgacgtagcttatcttcatgtatcaagtataaaattataatgagacatctattcacaaatacaagtcttagattgaattgaaatgtgttatagtgccctgtctcccactgacttgttcagttaaatgtcttaaagtacattatgtacatcttcaggcttttggtaccacaatggcacaagtatggtagggaggcaatatagtcttaggctatatgcctatattaagtgtgtataaacaatttttgaaagaatacactattatagatgtatgtgagtgatgctgacctgacagccatatccagtggatgaaactgactggacacactgttaaaatgttttaaagatgtattttcagccagaacagcctggttatagtttgtggttttcaccttggtggattgcaggaacacatgcagcctactggcattgagcattagctaatggcatgaaagggcctcatctcactacctctctaaggcctctagctccaagaaaaccatgaaaacttctttcttggagagatctttgtctcagaatccttagagaggatttcgtatgggggctaactttaggaagggaggcagctggggcaggactttctgatacctgacagtcatgttccagagcaacctttgggcagtggaaactggcgcatctatgcaaaatgattgctcaatctctatcttgtgtactacatatgtaactagctgggccctaaggaaggttttctagggggaaggatagggaagtagaggaggagacaagtaggaggaacaaagcattctagacccaagaggatagaagatatttaggatagatatggctttcatccatagttcaaaataatgcgttttgttagatgccagttatagcagtaaataggttatagtttttatatgtcaagatttacctgtaatcagactcattctttcactctctatacccactgtctccatgcttgggagcatggatattaatagttccagtgatgtagaagttagtgatttttgatttctgaaaaaggtgagaaccttttattacagttggagaatatttgtcaaaaattcaaaggttgttgtaattgagttgccagaattacagagtttccattttcagatatcacagttgaatcacctctgtagattgttataaagagaggcattttaagatagtattttatttgctaggttgtgtctcagtctaagaattgggaaaagaagagctataggtttctctttcctagtctggatttcagtaaacacaagcctacctctgcttctttggttcacagcagtgtggatcatgaaatgaactgtttacccacattcatcaatattggtattttacaaatctacttggagcatttaatttcatctcaaagattgtgatccactttagataagcacaaatacagtattaggaaaagtaaatatgcaatcttactaaaatttcaacttgttaagctgtatatcttaaaagaaattatttggg

SEQ ID NO: 36 - DNA, 25 bp, Artificial
  Bacteria Artifical Chromosome from Human Genomic DNA
taaatattgttttcacccaaaggaa

SEQ ID NO: 37 - DNA, 25 bp, Artificial
  Bacteria Artificial Chromosome from Human Genomic DNA
tgtgcttatctaaagtggatcacaa

SEQ ID NO: 38 - DNA, 1760 bp, Artificial
  Bacteria Artifical Chromosome from Human Genomic DNA
caacattttttaaaagagaaaaagcgtgcacattgttttaacagttaggtcttgagaatattatgattaatgttgcgagacaggaagggagacacacactgacatgaacacctagagacagagtgcagctgttgtgtgcacgggtggctgagctctcaaattcactgtggaatcaaactgttggcaagtgaaagtgatggagttggagcatcctccttgatctgaaagccatgcgtgtgaggaaagactgaaagaccagggcatgttcagtcctacagggataaagtttaagggagacctggctgttgtccaacactgcagggctgccaggaggaagaaggagctgaatgttacagctgactgattggggcactgggtgaaagacaccagaagatagattggggccctgtgggagggcttcctcccgctaggagctgctccataatgctggggcaaacttgaaagaacagttcagccagaattgtggtggtcatgagtagagaggctgtgaaaaggatttctctctgattattgattaggaggttggataaggagacttccaagatcccttgcaacgctgagattctatacaacccacctgatctctgcaatccattcctgggtttggttgaaaagggaagcatgtcccattttccgtggttatctctcctgcctcctcttacagaagtctttatttcatcattaacttatttattttctgatcaaatagctcagattgtacattagtcatcatttttaagtgccattgtaggtagtactgatatgttctattcaaaaggaaaactgtcacatgtattggggtctctaaggtgaattggaaattatccaggtatgttctgcatatggaaaatgctcatttaaataattgctggatgaagaagtactgaatgaatgaaaatgaacaattaaagcagcaagatgattatttacaaatggtccttagttaaagccaatgagccatcactctttacatttaattagagaaaatctgtaggaagagctcggacttcagtggaacagagagcaggaacttgacaaaaaaaaattattgggttgagatttggaaaaagaaataaaaaaagctgtagatgggatgtacagagaggaaatctgttactaatttcagatgtcctgaataaagctgctaataacaaatcattactaattactaatcttgtcattactattcctggagatagtgttatgggaaatgggaatagccatggcagacagtattcttgtatgttagcatttagagtgattctgttttccagcatcggtcatacagacgggtcatctggccaaactgcaaaagctggacctgagctacaatgacagcatctgtgatgcggggtggaccatgttctgccaaaacgtgcggttcctcaaagagctaatcgagctggatattagccttcgaccatcaaattttcgagattgtggacaatggtttagacacttgttatatgctgtgaccaagcttcctcagatcactgagataggaatgaaaagatggattctcccagcttcacaggaggaagaactagaatgctttgaccaagataaaaaaagaagcattcactttgaccatggtgggtttcagtaaactgatttcccatgtcctactaagctacaaaccattctccaaaggaaaagaacatgaacgaattccagagtcatgaactgaatttcaacttctgggccatttaatgggacttatattacaagagctttgtaaa

SEQ ID NO: 39 - DNA, 23 bp, Artificial
  Bacteria Artificial Chromosome from Human Genomic DNA
taaaagagaaaaagcgtgcacat

SEQ ID NO: 40 - DNA, 22 bp, Artificial
  Bacteria Artifical Chromosome from Human Genomic DNA
taaatggcccagaagttgaaat

SEQ ID NO: 41 - DNA, 3874 bp, Artificial
  Bacteria Artificial Chromosome from Human Genomic DNA
agaatacttaaaaaaactagtacatgtacattatttcaactactcacacaatttctgtaggactgaaacacagaaattagctactgttggaatgaaactgatagaaccattacctttgaaaaaaggttttctaaaatttgtctatcaaaatcatcctgtgaaattaaacataagacaaatgccatttaaaaacagcaaaaaaatttttatataatctacggtttcttttaaaatagtctaaaatctaacatacagtaggcatgatctgatacaaatacaaaagaaaattaaatttatttagaaaatagtttctcagtaagatcttcaccttctaaagacttccataaaaatatgtaatacttaaataattactaatagacctttcccaaaacactgcattgtaacaaatataaagttggacttaactaaaatggtagttatgagtgttagaatgataccagatacaactaaatatatgcatattatgataaataatagcatagcattaagagaagaatgccaaagaagggatagtatcttcaagttttctctacacatcattctagaagagcagaaaatgatgacaactcaagccatgtcaatttaaataaaattactttagctaagaagcagaaaatgtgatcaagaaaagctcaatcaatcttagccacttcaatcgtatcaaataatactaaaaataataaatttgaaggctaatctatgcaaaggcagaatgctaataataccagaggaaatttggcaactattgcccttatagaattatgacgtcactgtagcagttttttgttcattagatgcttggattaacgaagatataaataaattaaacataacctctccattttagcaaacttcctaaagaggctgagtcttttaattttctataatttcttcacattccaatacattcctatcacatttaacccagaagagattaggtaacattctaatgctttgtaaattgtatttaaacccactattttaacttgatacacatgcatagatttcttagataaaataatcaaatattgtttgcatcatacaaaggtggtatttcaaactctattcaaataagtcagtcccatgtcccaccagtggcaatttattatgaaaataatgtgtgtgctgtgaaatggtgaagatctctacagactacaagcattaagtgtttttattatgtcctctaattggttcttatgacttgggtcccaaatagagaaaattaatgaattaaagaaatcaataccaataaaaaacatattaactgtactacaggtgtagtttaaaagcagaacacaaaaaagccacagcatggcagaaataagaaatgatgtcatctcacactgaacaacagccttgtggggggaaaaaaaaagaaaaagaatatcaccatgctgtttgattggcactataaaaattgactttgtggaactcataatttcatatccaaagatcaggcagtgaaataaattaatatgtgggaaaagtggcatggggatgagtggtgcaaatatcaggataataacgaattttctacattcatttggaaggaaaaggaccattgtgaaactacaaccaccaccaaaagttgtggtttaatttataaaattaacaaaccccaaacttatagaaaactcagatataaaatatttaactatagttggctttctggtaactcaaaatgtttacttataattgaggttaatttcttaactctttatgcaatataatgtaccaaatttagaagccaagtgatttctagtatgcattaagtttatatctagaaaattctttctggaattcagtatttattttaaaaagtgcataacaggagtaagagaggcttaccaaaatatttcaaaactcaagtacattttcctcatctccaatattttagaacggaataaaacataaactgaatcaaattttgaaactgtctttgacaagataaaaacaccaaatatctacttaaaattcattttgaaaatacaatctcagataactcaaaccaaaagtgctgaccattggttttctccagaatactcagttctaaatgaacaaaaatttatatgcacttttctagattttgagaaacatttttgtttcattagaaaagttatttctaacaatatattaatagagaaatagcacaattcttattcagcgcctaaatttttacgagcgaaaatatgaaattttatttttatgcatagtttatgtattgatccatggggcttacaaatagcaacacactcttgggctgatactatcgtggattttgcttaaattatgagggcaggaaaattttaaaatcccacaggtcacaactgaatcacattaactggctgatttagtaaatgaagggcaattctaaaatgcaaaataaaaatggaattaaggcagctttaaaagaaaataaaactcatccacccaaaatagtgctacataattcattacttaaaaagctctctgtggagtatagacataaagccaaaaataaaaacaaacattgcagttgtgatgcagcatcaggtgcttttacttcagtgaatgaaaaataatggtcacaactcaaatgaatgggaatttaatatgaatatatgcaccttaccagagatgtttgctaccaatgatatcttagcaattccatattccttacaaagtcagtataattgttgtaaaaaaatcaactgtggttctgaatacccattcacagttgacctcaacaatgtatctgatgtaggagactgagtatccgtgacaggcagaagcatgtgatggtcctcagtcccaagtggaagagctaatggtaaagtcatatcagaaggcttcacatccatagtttctgataaaggacttttttgtatggaatcctgttcactcaaagtatgatcctctgcactggagtctagagttttatctgcacctagaatggaaggaaaaaaaaatctacattatctttaaaacaaaactaaacccctgaggtagactaatgtacttaccattcccagaaattacattccaaccaccatacctttactcaagcggtttctcttttgtacatatgccctcctcactatctatgtaaattcaatctgggcctcaaggttaagttcaagcaccaactccatttaaaaaaatggacttgattaagaatgaaacagggtgagctgttatatcggcttaatcacacatttgtcaattttcatatactgccttgtggcatctatctctcttattgccatttaacattctactcaggtatgccttgaatcctcaaccagactgtaaattctttgagagtaaatgtcgttatacttctttaactccctcaaaatattaaacagtgataagaaatactcttttcattcactgaatgtttttaataagttgttcatattattctgtgagttcagaacagtatgtatggcactttcatatattttcagaggtgagaaattctgctctactcaagggagcaatcagcacactgactaaagatgtacttttgccttcaaattttccaatggctttaacatgcttccgttactctaaagtgttgacatgattttggtttcaccgtccttgtcacgcagacaaaactgctttcgtaaaattgtaattatctgtattcttcccaaactactcttcacttcaactagctaaggtttccttcttgtcccactgactctctacagtgttctctgcccggaatgaatcctctcttcccatctccatctttgaactcttctccatccttcaagttaaaattaagttttttttagtacttacctcagaaaaacatagtatcttctttagcacttttgtgtcgattatgccctacattttccct

SEQ ID NO: 42 - DNA, 25 bp, Artificial
  Bacteria Artificial Chromosome from Human Genomic DNA
gttggaatgaaactgatagaaccat

SEQ ID NO: 43 - DNA, 23 bp, Artificial
  Bacteria Artifical Chromosome from Human Genomic DNA
gtagggcataatcgacacaaaag

SEQ ID NO: 44 - DNA, 52 bp, Mycoplasma sp.
gatgatggcagcggccatgctagggattgattcttgtccgattgaagggtat

SEQ ID NO: 45 - DNA, 50 bp, Mycoplasma fermentans
aaaagtgagcgccttggttcaattgaaagactcactaaccaagaaaaatt

SEQ ID NO: 46 - DNA, 50 bp, Mycoplasma sp.
tcctacgggaggcagcagtagggaattttcggcaatgggggaaaaccctg

SEQ ID NO: 47 - DNA, 50 bp, Mycoplasma hominis
cgtaaacgatgatcattagtcggtggagaatcactgacgcagctaacgca

SEQ ID NO: 48 - DNA, 50 bp, Mycoplasma hyorhinis
aacattagttagttggtagggtaatggcctaccaagacgatgatgtttag

SEQ ID NO: 49 - DNA, 50 bp, Mycoplasma arginini
cgtaaacgatgatcattagtcggtggagagttcactgacgcagctaacgc

SEQ ID NO: 50 - DNA, 50 bp, Mycoplasma orale
cgctgtaaacgatgatcattagtcggtggaaaactactgacgcagctaac

SEQ ID NO: 51 - DNA, 50 bp, Acheoplasma laidlawii
cgatgagaactaagtgttgggcaaaaggtcagtgctgcagttaacgcatt

SEQ ID NO: 52 - DNA, 50 bp, Mycoplasma salivarium
attaactggaactatgctaactgcattgggccaaacatattctggaattt

SEQ ID NO: 53 - DNA, 50 bp, Mycoplasma pulmonis
atgtcgagcggagtattaatttattagtgcttagcggcaaatgggtgagt

SEQ ID NO: 54 - DNA, 50 bp, Mycoplasma pneumoniae
aacaaaacgcaagcttacgattccagtagcattaagattcttgaaggctt

SEQ ID NO: 55 - DNA, 46 bp, Mycoplasma pirum
ccctcatcctatagcggtccaaacggacctttaaaatgtttctcat

SEQ ID NO: 56 - DNA, 50 bp, Mycoplasma capricolum
ttgtcctattgaaacaccagaaggaccaaacattggattaattaataact

SEQ ID NO: 57 - DNA, 50 bp, Helicobacter pylori
gggactagcgttaaacgcacgaagaatttgatgaaagttcctgttggcga